SEQUENCE LISTING (107 sequences)

SEQ ID NO: 1 - protein, 735 aa, Artificial Sequence [sequence omitted]

SEQ ID NO: 2 - DNA, 2208 bp, Artificial Sequence
  Synthetic Construct
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 3 - protein, 736 aa, Artificial Sequence
  Synthetic Construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - DNA, 2211 bp, Artificial Sequence
  Synthetic Construct
atggctgccgatggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 5 - protein, 735 aa, Artificial Sequence
  Synthetic Construct
MAADGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 6 - DNA, 2208 bp, Artificial Sequence
  Synthetic Construct
atggctgctgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 7 - protein, 724 aa, Artificial Sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 8 - DNA, 2175 bp, Artificial Sequence
  Synthetic Construct
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaagcaccagacctatcggaacccgataccttacccgacccctttaa

SEQ ID NO: 9 - protein, 736 aa, Artificial Sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 10 - DNA, 2211 bp, Artificial Sequence
  Synthetic Construct
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 11 - protein, 737 aa, Artificial Sequence
  Synthetic Construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 12 - DNA, 2214 bp, Artificial Sequence
  Synthetic Construct
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 13 - protein, 738 aa, Artificial Sequence
  Synthetic Construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 14 - DNA, 2217 bp, Artificial Sequence
  Synthetic Construct
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 15 - protein, 736 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - DNA, 2211 bp, Artificial Sequence
  Synthetic Construct
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 17 - protein, 738 aa, Artificial Sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 18 - DNA, 2484 bp, Artificial Sequence
  Synthetic Construct
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaattgcttgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactag

SEQ ID NO: 19 - protein, 736 aa, Artificial Sequence
  Synthetic Construct
MSFVDHPPDWLESIGDGFREFLGLEAGPPKPKANQQKQDNARGLVLPGYKYLGPGNGLDKGDPVNFADEVAREHDLSYQKQLEAGDNPYLKYNHADAEFQEKLASDTSFGGNLGKAVFQAKKRILEPLGLVETPDKTAPAAKKRPLEQSPQEPDSSSGVGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGSSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 20 - DNA, 2211 bp, Artificial Sequence
  Synthetic Construct
atgtcttttgttgaccaccctccagattggttggaatcgatcggcgacggctttcgtgaatttctcggccttgaggcgggtcccccgaaacccaaggccaatcaacagaagcaagataacgctcgaggtcttgtgcttcctgggtacaagtatcttggtcctgggaacggccttgataagggcgatcctgtcaattttgctgacgaggttgcccgagagcacgacctctcctaccagaaacagcttgaggcgggcgataacccttacctcaagtacaaccacgcggacgcagagtttcaggagaaactcgcttctgacacttcttttgggggaaaccttgggaaggctgttttccaggctaaaaagaggattctcgaacctcttggcctggttgagacgccggataaaacggcgcctgcggcaaaaaagaggcctctagagcagagtcctcaagagccagactcctcgagcggagttggcaagaaaggcaaacagcctgccagaaagagactcaactttgacgacgaacctggagccggagacgggcctcccccagaaggaccatcttccggagctatgtctactgagactgaaatgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggctcgagcaacgccagcgacaccttcaacggattctccaccccctggggatactttgactttaaccgcttccactgccacttctcgccaagagactggcaaaggctcatcaacaaccactggggactgcgccccaaaagcatgcaagtccgcatcttcaacatccaagttaaggaggtcacgacgtctaacggggagacgaccgtatccaacaacctcaccagcacggtccagatctttgcggacagcacgtacgagctcccgtacgtgatggatgcaggtcaggagggcagcttgcctcctttccccaacgacgtgttcatggtgcctcagtacgggtactgcggactggtaaccggaggcagctctcaaaaccagacagacagaaatgccttctactgtctggagtactttcccagccagatgctgagaaccggaaacaactttgagatggtgtacaagtttgaaaacgtgcccttccactccatgtacgctcacagccagagcctggataggctgatgaacccgctgctggaccagtacctgtgggagctccagtctaccacctctggaggaactctcaaccagggcaattcagccaccaactttgccaagctgaccaaaacaaacttttctggctaccgcaaaaactggctcccggggcccatgatgaagcagcagagattctccaagactgccagtcaaaactacaagattccccagggaagaaacaacagtctgctccattatgagaccagaactaccctcgacggaagatggagcaattttgccccgggaacggccatggcaaccgcagccaacgacgccaccgacttctctcaggcccagctcatctttgcggggcccaacatcaccggcaacaccaccacagatgccaataacctgatgttcacttcagaagatgaacttagggccaccaacccccgggacactgacctgtttggccacctggcaaccaaccagcaaaacgccaccaccgttcctaccgtagacgacgtggacggagtcggcgtgtacccgggaatggtgtggcaggacagagacatttactaccaagggcccatttgggccaaaattccacacacggatggacactttcacccgtctcctctcattggcggatttggactgaaaagcccgcctccacaaatattcatcaaaaacactcctgtacccgccaatcccgcaacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctag

SEQ ID NO: 21 - protein, 727 aa, Artificial Sequence
  Synthetic Construct
MSFVDHPPDWLEEVGEGLHEFLELEAGPPKPKPNQQKQDNARGLVLPGYNYLGPFNGLDKGEPVNRADAVAREHDISYNEQLQAGDNPYLKYNHADAEFQEKLKDDTSFGGNLGKAIFQAKKRVLEPFGLVEAPVKTAPAKKRPIEKSPAEPSSSKGIGKAGQQPARKRLNFGQTGDTDSAADPQPLGEPPAAPSGLGTGTMASGGGAPMADNNEGADGVGNASGNWHCDSTWLGDRVITTSTRTWALPTYNNHLYKQISSSNYGANNDNHYFGYSTPWGYFDFNRFHCHFSPRDWQRLINNNWGFRPKRLNFKLFNIQVKEVTETDGTKTIANNLTSTVQVFADSEYQLPYVLGSAHQGCFPPFPADVFMIPQYGYLTLNNGSQAMGRSSFYCLEYFPSQMLRTGNNFTFSYTFEDVPFHSSYAHSQSLDRLMNPLIDQYLYYLSKTAGGLAFSQAGPTTMRNQSRNWLPGPCFRQQRVSERSTENNNGDFSWTGTTRYHLNGRDSAMNPGPAMASHKDDEDRFFPQNGVLIFGTPNATASNAPLENVLITDEEEIRTTNPVATEEYGIVANNKQDSSTQATTAIVNAQGILPGMVWQDRDVYLQGPIWAKIPHTDGHFHPSPLMGGFGLKHPPPQILIKNTPVPSNPPEKFTQEKLNAFITQYSTGQVSVEIEWELQKENSKRWNPEVQYTSNYNKSVNVDFTVDTNGMYSEPRTIGTRYLTRNL

SEQ ID NO: 22 - DNA, 2184 bp, Artificial Sequence
  Synthetic Construct
atgtcgtttgttgatcaccctccagattggttggaagaggttggtgaaggccttcacgagtttttggagctcgaagctggcccacccaaaccgaagcccaaccagcagaagcaggacaacgcccgtggtcttgtactgcctggatataattatctgggacccttcaacggactcgacaagggagagcccgtcaaccgagcggacgctgttgcgcgagagcacgacatctcgtacaacgagcagcttcaggcgggagacaacccctacctcaagtacaaccacgcggacgccgagtttcaggagaagctcaaggacgacacctcctttgggggcaacctcggaaaggcaatctttcaggccaagaaaagggttctcgaaccttttggcctggttgaagcgcctgttaagacggctccagccaagaagcggccgatagagaagtctccggcggaaccgagctcttcgaagggcatcggcaaggcgggccagcagcctgcgagaaagcggctcaactttggtcagactggagacaccgactccgccgctgacccccagcctctcggagaaccacccgcagccccctctggtctgggaactggtacgatggcttcaggcggtggcgcaccaatggcagacaataacgaaggcgccgacggagtgggtaatgcctcgggaaattggcattgcgattccacatggctgggcgaccgagtcatcaccaccagcacccgcacctgggccctgcccacctacaacaaccacctctacaagcaaatctcctctagcaattacggagccaacaacgacaaccactactttggctacagcaccccctgggggtattttgacttcaaccgtttccactgccacttctctccgcgagactggcagcggctcatcaacaacaactgggggttccggcccaagcgactcaacttcaagctgttcaacatccaagtcaaggaggtcacagaaacggacggcacgaagacgatcgccaataaccttaccagcacggttcaagtctttgcggactcggagtaccagctgccgtatgtcctcggctcggcacaccagggctgcttcccgccgttcccagcggacgtctttatgatcccacaatacggatacctgacgctgaacaacggcagccaggcgatgggtcgctcttccttctactgcctggaatactttccgtcgcagatgctgcggacgggaaacaacttcacgttcagctacacctttgaggacgtgcccttccacagcagctacgcgcacagccagagcctggaccggctgatgaacccgctcatcgaccagtacctgtactacctgagcaagacagccggtggtctggcgttttctcaagcagggcccaccaccatgcggaaccagtccaggaactggctcccgggaccctgcttccgacagcagcgagtttcagaaagatccacagaaaacaacaacggagacttctcgtggacagggaccacgagataccatcttaacggtagagactcggcgatgaatccgggcccggccatggccagtcacaaggacgacgaggacagattctttccgcaaaacggcgtgctcatctttggaacaccgaacgctactgcaagcaatgcacctctagagaatgtactgatcaccgatgaggaggaaatcagaactacgaaccctgtagccacggaagagtacgggatcgttgctaacaacaaacaggacagcagtacacaggcgacgaccgcgattgtcaacgcacaaggcatactgcccggcatggtgtggcaggatcgagatgtgtacctgcaggggcccatctgggccaagatcccccacacggatggacacttccacccatcaccgctcatgggcgggtttggccttaagcatccacctccgcagatcctcatcaagaacacgcctgtgccgtcgaaccctccagaaaagttcacccaggaaaaactaaatgccttcatcacacagtactcgacgggccaggtcagcgtggagatcgagtgggagctgcagaaggagaacagcaagcgctggaacccagaggtccagtacacgtccaactacaacaagtccgtcaacgtggactttactgtggacaccaacggcatgtactcggagccccgcactatcggcacccgctacctcacccgcaacctgtaa

SEQ ID NO: 23 - protein, 716 aa, Artificial Sequence
  Synthetic Construct
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEDPVKTAAKGERIDDHYPKKKKARVEETEAGTSGAQQLQIPAQPASSLGADTMSAGGGSPLGDNNQGADGVGNASGDWHCDSTWMGDRVITKSTRTWVLPSYNNHLYKEIHNNGVDGSTANAYFGYSTPWGYFDFNRFHSHWSPRDWQRLINNYWGFRPRSLKVKIFNIQVKEVTVQDNTTTIANNLTSTVQVFTDNDYQLPYVIGNGTEGCLPAFPPQVFTLPQYGYATLNRNNSENPTERSSFFCLEYFPSKMLRTGNNFEFTYSFEEVPFHCSFAPSQNLFKLANPLVDQYLYRFVSTDTSGAVQFKKNLAGRYANTYKNWFPGPMCRTQGWYTGTGIYNNKGATSFNTSNRMDLEGASYQVPPQPNGMTNTVQDSNLYALENTMIFNAQNATPGTNTTYPEENLLITSESETQPVNRVAYSSGGQIANNNQNTNTAPTAGTYNHQEILPGSVWMDRDVYLQGPIWAKIPETGAHFHPSPAMGGFGLKYPPPMMLIKNTPVPGNITTFSDVPVQSFITQYSTGQVTVEMEWELKKENSKRWNPEIQYTNNYNNPTFVDFAPDTEGEYRTTRAIGTRYLTRPL

SEQ ID NO: 24 - DNA, 2151 bp, Artificial Sequence
  Synthetic Construct
atgtcgtttgttgatcaccctccagattggcttgaggagattggtgagggtctaaaggagtttttgggactcgaacctggcccacccaaaccgaagcccaaccagcagaagcaagacaacgcccgtggtcttgtactgcctggatataattacctgggacccggcaacggtctcgaccgcggagaacctgtcaaccgggccgacgaggtcgcgcgagagcacgacatctcgtacaacgagcagctccaggcgggagacaacccctacctcaagtacaaccacgcggacgccgagtttcaagagaagctcgcggacgacacctccttcgggggcaacctcggcaaggcagtctttcaggccaagaaaagggttctcgaaccttttggcctggttgaggatcctgttaagacggctgctaaaggcgagcggatagacgaccactatcccaagaagaagaaggctcgggttgaagaaaccgaagctggaaccagcggagcccagcagctgcagatcccagcccaaccagcctcaagtttgggagctgatacaatgtctgcgggaggtggcagcccactgggcgacaataaccaaggcgccgatggagtgggcaatgcctcgggagattggcattgcgattccacatggatgggggacagagtcatcaccaagtccacccgaacctgggtgctgcccagctacaataaccacctgtacaaagagatccacaacaacggggtcgacggcagcaccgccaacgcttactttggatacagtaccccctgggggtactttgacttcaaccgcttccacagccactggagccctcgagactggcagcgactcatcaacaactactggggcttcagaccccggtccctcaaggtcaagattttcaacatccaagtcaaagaggtcacggtgcaggacaacaccaccaccatcgccaacaacctcacctccaccgtccaagtgtttacggacaacgactaccagctgccgtacgtcatcggcaacgggaccgaggggtgtctaccggccttccctccgcaggtctttacgctgccgcagtatggctacgcgacgctgaatcgcaacaatagcgaaaatcccaccgagcgaagcagcttcttctgtctggagtactttcccagcaagatgctgcggacgggcaacaactttgagttcacatacagcttcgaggaggtacccttccactgcagcttcgcgcccagccaaaacctcttcaaactggctaacccgttggtggatcagtacctttaccgcttcgtgagcacggacacctccggtgccgtccagttcaaaaagaacctggcgggcagatacgccaacacctacaagaactggttcccaggacccatgtgccgaacccagggctggtacacaggaacgggtatatataacaacaaaggcgctaccagctttaacacctcaaacagaatggacctagagggagccagttatcaagtgcctccccagcccaacgggatgacaaacacggttcaggacagcaacctttacgcgctggaaaacaccatgatttttaacgcacaaaacgccaccccgggaacgaatacaacgtatccggaggagaaccttttgataaccagtgagagcgagactcaacccgtgaacagagtggcttacagctccggaggacaaatagccaacaacaatcagaataccaacacggctcctactgcaggaacctacaaccaccaggaaatactacctggcagcgtgtggatggacagggacgtgtacctccagggtcccatctgggccaagatcccagagacaggggcacactttcatccttctccagccatgggcgggttcggactcaaatacccgcctcccatgatgctaatcaagaacacgccagtgcccggaaacatcaccaccttctcggacgtgcccgtccaaagttttattacccagtacagcaccggacaagtcaccgtggagatggagtgggagctcaagaaggaaaactctaagaggtggaaccccgagatacagtacaccaacaactacaacaaccctacgttcgtggactttgctccagacacagaaggagaatacaggaccactagggctattggaacccgctaccttacccgacctctgtaa

SEQ ID NO: 25 - protein, 737 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - DNA, 2214 bp, Artificial Sequence
  Synthetic Construct
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 27 - protein, 738 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - DNA, 2217 bp, Artificial Sequence
  Synthetic Construct
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 29 - protein, 742 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - DNA, 2229 bp, Artificial Sequence
  Synthetic Construct
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 31 - protein, 734 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - DNA, 2160 bp, Artificial Sequence
  Synthetic Construct
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctgatcgaccagtacctgtactacctgagcaagacagccggtggtctggcgttttctcaagcagggcccaccaccatgcggaaccagtccaggaactggctcccgggaccctgcttccgacagcagcgagtttcagaaagatccacagaaaacaacaacggagacttctcgtggacagggaccacgagataccatcttaacggtagagactcggcgatgaatccgggcccggccatggccagtcacaaggacgacgaggacagattctttccgcaaaacggcgtgctcatctttggaacaccgaacgctactgcaagcaatgcacctctagagaatgtactgatcaccgatgaggaggaaatcagaactacgaaccctgtagccacggaagagtacgggatcgttgctaacaacaaacaggacagcagtacacaggcgacgaccgcgattgtcaacgcacaaggcatactgcccggcatggtgtggcaggatcgagatgtgtacctgcaggggcccatctgggccaagatcccccacacggatggacacttccacccatcaccgctcatgggcgggtttggccttaagcatccacctccgcagatcctcatcaagaacacgcctgtgccgggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 33 - protein, 737 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - DNA, 2214 bp, Artificial Sequence
  Synthetic Construct
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 35 - protein, 737 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - DNA, 2214 bp, Artificial Sequence
  Synthetic Construct
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 37 - protein, 737 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - DNA, 2214 bp, Artificial Sequence
  Synthetic Construct
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 39 - protein, 743 aa, Artificial Sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 40 - DNA, 2232 bp, Artificial Sequence
  Synthetic Construct
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggcccttgagcacgacaaggcctacgaccagcagctcaaagcgggtgacaatccgtacctgcggtataaccacgccgacgccgagtttcaggagcgtctgcaagaagatacgtcttttgggggcaacctcggacgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggcagcaggcggtggcgctcccatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcctgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 41 - protein, 737 aa, Artificial Sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 42 - DNA, 2214 bp, Artificial Sequence
  Synthetic Construct
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 43 - protein, 733 aa, Artificial Sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVTVEMEWELKKENSKRWNPEIQYTNNYNDPQFVDFAPDSTGEYRTTRPIGTRYLTRPL

SEQ ID NO: 44 - DNA, 2202 bp, Artificial Sequence
  Synthetic Construct
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagatggtgtacaagtttgaaaacgtgcccttccactccatgtacgctcacagccagagcctggataggctgatgaacccgctgctggaccagtacctgtgggagctccagtctaccacctctggaggaactctcaaccagggcaattcagccaccaactttgccaagctgaccaaaacaaacttttctggctaccgcaaaaactggctcccggggcccatgatgaagcagcagagattctccaagactgccagtcaaaactacaagattccccagggaagaaacaacagtctgctccattatgagaccagaactaccctcgacggaagatggagcaattttgccccgggaacggccatggcaaccgcagccaacgacgccaccgacttctctcaggcccagctcatctttgcggggcccaacatcaccggcaacaccaccacagatgccaataacctgatgttcacttcagaagatgaacttagggccaccaacccccgggacactgacctgtttggccacctggcaaccaaccagcaaaacgccaccaccgttcctaccgtagacgacgtggacggagtcggcgtgtacccgggaatggtgtggcaggacagagacatttactaccaagggcccatttgggccaaaattccacacacggatggacactttcacccgtctcctctcattggcggatttggactgaaaagcccgcctccacaaatattcatcaaaaacactcctgtacccgccaatcccgcaacgaccttctctccggccagaatcaacagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 45 - protein, 720 aa, Artificial Sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFTFSYTFEDVPFHSSYAHSQSLDRLMNPLIDQYLYYLSKTAGGLAFSQAGPTTMRNQSRNWLPGPCFRQQRVSERSTENNNGDFSWTGTTRYHLNGRDSAMNPGPAMASHKDDEDRFFPQNGVLIFGTPNATASNAPLENVLITDEEEIRTTNPVATEEYGIVANNKQDSSTQATTAIVNAQGILPGMVWQDRDVYLQGPIWAKIPHTDGHFHPSPLMGGFGLKHPPPQILIKNTPVPSNPPEKFTQEKLNAFITQYSTGQVTVEMEWELKKENSKRWNPEIQYTNNYNDPQFVDFAPDSTGEYRTTRPIGTRYLTRPL

SEQ ID NO: 46 - DNA, 2163 bp, Artificial Sequence
  Synthetic Construct
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttacgttcagctacacctttgaggacgtgcccttccacagcagctacgcgcacagccagagcctggaccggctgatgaacccgctcatcgaccagtacctgtactacctgagcaagacagccggtggtctggcgttttctcaagcagggcccaccaccatgcggaaccagtccaggaactggctcccgggaccctgcttccgacagcagcgagtttcagaaagatccacagaaaacaacaacggagacttctcgtggacagggaccacgagataccatcttaacggtagagactcggcgatgaatccgggcccggccatggccagtcacaaggacgacgaggacagattctttccgcaaaacggcgtgctcatctttggaacaccgaacgctactgcaagcaatgcacctctagagaatgtactgatcaccgatgaggaggaaatcagaactacgaaccctgtagccacggaagagtacgggatcgttgctaacaacaaacaggacagcagtacacaggcgacgaccgcgattgtcaacgcacaaggcatactgcccggcatggtgtggcaggatcgagatgtgtacctgcaggggcccatctgggccaagatcccccacacggatggacacttccacccatcaccgctcatgggcgggtttggccttaagcatccacctccgcagatcctcatcaagaacacgcctgtgccgtcgaaccctccagaaaagttcacccaggaaaaactaaatgccttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 47 - protein, 725 aa, Artificial Sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTDTSGAVQFKKNLAGRYANTYKNWFPGPMCRTQGWYTGTGIYNNKGATSFNTSNRMDLEGASYQVPPQPNGMTNTVQDSNLYALENTMIFNAQNATPGTNTTYPEENLLITSESETQPVNRVAYSSGGQIANNNQNTNTAPTAGTYNHQEILPGSVWMDRDVYLQGPIWAKIPETGAHFHPSPAMGGFGLKYPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 48 - DNA, 2178 bp, Artificial Sequence
  Synthetic Construct
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggatcagtacctttaccgcttcgtgagcacggacacctccggtgccgtccagttcaaaaagaacctggcgggcagatacgccaacacctacaagaactggttcccaggacccatgtgccgaacccagggctggtacacaggaacgggtatatataacaacaaaggcgctaccagctttaacacctcaaacagaatggacctagagggagccagttatcaagtgcctccccagcccaacgggatgacaaacacggttcaggacagcaacctttacgcgctggaaaacaccatgatttttaacgcacaaaacgccaccccgggaacgaatacaacgtatccggaggagaaccttttgataaccagtgagagcgagactcaacccgtgaacagagtggcttacagctccggaggacaaatagccaacaacaatcagaataccaacacggctcctactgcaggaacctacaaccaccaggaaatactacctggcagcgtgtggatggacagggacgtgtacctccagggtcccatctgggccaagatcccagagacaggggcacactttcatccttctccagccatgggcgggttcggactcaaatacccgcctcccatgatgctaatcaagaacacgccagtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 49 - protein, 6 aa, Artificial Sequence
  Synthetic Construct
FTFSYT

SEQ ID NO: 50 - protein, 6 aa, Artificial Sequence
  Synthetic Construct
FEITYS

SEQ ID NO: 51 - protein, 6 aa, Artificial Sequence
  Synthetic Construct
FQFTYT

SEQ ID NO: 52 - protein, 6 aa, Artificial Sequence
  Synthetic Construct
  MISC_FEATURE (2)..(2): Xaa can be E, Q, or T
  MISC_FEATURE (3)..(3): Xaa can be F, I or M
  MISC_FEATURE (4)..(4): Xaa can be S, T or V
  MISC_FEATURE (6)..(6): Xaa can be E, K,  N, Q, S or T
FXXXYX

SEQ ID NO: 53 - protein, 6 aa, Artificial Sequence
  Synthetic Construct
  MISC_FEATURE (2)..(2): Xaa can be E, Q, or T
  MISC_FEATURE (3)..(3): Xaa can be F or I
  MISC_FEATURE (4)..(4): Xaa can be S or T
  MISC_FEATURE (6)..(6): Xaa can be E, N, Q, S or T
FXXXYX

SEQ ID NO: 54 - protein, 6 aa, Artificial Sequence
  Synthetic Construct
FQFSYT

SEQ ID NO: 55 - protein, 6 aa, Artificial Sequence
  Synthetic Construct
FEFTYN

SEQ ID NO: 56 - protein, 6 aa, Artificial Sequence
  Synthetic Construct
FTFSYT

SEQ ID NO: 57 - protein, 6 aa, Artificial Sequence
  Synthetic Construct
FEFSYS

SEQ ID NO: 58 - protein, 6 aa, Artificial Sequence
  Synthetic Construct
FQFSYE

SEQ ID NO: 59 - protein, 6 aa, Artificial Sequence
  Synthetic Construct
FEFSYQ

SEQ ID NO: 60 - protein, 6 aa, Artificial Sequence
  Synthetic Construct
FEMVYK

SEQ ID NO: 61 - protein, 6 aa, Artificial Sequence
  Synthetic Construct
FTFSYT

SEQ ID NO: 62 - protein, 6 aa, Artificial Sequence
  Synthetic Construct
FEFTYS

SEQ ID NO: 63 - protein, 7 aa, Artificial Sequence
  Synthetic Construct
QILIKNT

SEQ ID NO: 64 - protein, 7 aa, Artificial Sequence
  Synthetic Construct
QIFIKNT

SEQ ID NO: 65 - protein, 7 aa, Artificial Sequence
  Synthetic Construct
  MISC_FEATURE (1)..(1): Xaa can be Q or M
  MISC_FEATURE (2)..(2): Xaa can be I or M
  MISC_FEATURE (3)..(3): Xaa can be L, M or F
XXXIKNT

SEQ ID NO: 66 - protein, 7 aa, Artificial Sequence
  Synthetic Construct
  MISC_FEATURE (2)..(2): Xaa can be I or M
  MISC_FEATURE (3)..(3): Xaa can be F, L or M
QXXIKNT

SEQ ID NO: 67 - protein, 7 aa, Artificial Sequence
  Synthetic Construct
QIMIKNT

SEQ ID NO: 68 - protein, 7 aa, Artificial Sequence
  Synthetic Construct
MMLIKNT

SEQ ID NO: 69 - protein, 17 aa, Artificial Sequence
  Synthetic Construct
TQYSTGQVSVEIEWELQ

SEQ ID NO: 70 - protein, 17 aa, Artificial Sequence
  Synthetic Construct
TQYSTGQVSVQIDWEIQ

SEQ ID NO: 71 - protein, 17 aa, Artificial Sequence
  Synthetic Construct
  MISC_FEATURE (9)..(9): Xaa can be A, S or T
  MISC_FEATURE (11)..(11): Xaa can be E, K or Q
  MISC_FEATURE (12)..(12): Xaa can be I or M
  MISC_FEATURE (13)..(13): Xaa can be D or E
  MISC_FEATURE (16)..(16): Xaa can be I or L
  MISC_FEATURE (17)..(17): Xaa can be Q or K
TQYSTGQVXVXXXWEXX

SEQ ID NO: 72 - protein, 17 aa, Artificial Sequence
  Synthetic Construct
  MISC_FEATURE (9)..(9): Xaa can be A, S or T
  MISC_FEATURE (11)..(11): Xaa can be E or K
  MISC_FEATURE (12)..(12): Xaa can be I or M
  MISC_FEATURE (16)..(16): Xaa can be I or L
  MISC_FEATURE (17)..(17): Xaa can be Q or K
TQYSTGQVXVXXEWEXX

SEQ ID NO: 73 - protein, 17 aa, Artificial Sequence
  Synthetic Construct
TQYSTGQVTVEMEWELK

SEQ ID NO: 74 - protein, 17 aa, Artificial Sequence
  Synthetic Construct
TQYSTGQVAVKIEWEIQ

SEQ ID NO: 75 - protein, 5 aa, Artificial Sequence
  Synthetic Construct
VPFHS

SEQ ID NO: 76 - protein, 5 aa, Artificial Sequence
  Synthetic Construct
KHPPP

SEQ ID NO: 77 - protein, 6 aa, Artificial Sequence
  Synthetic Construct
MKHPPP

SEQ ID NO: 78 - protein, 6 aa, Artificial Sequence
  Synthetic Construct
  MISC_FEATURE (5)..(5): Xaa can be E, Q, or T
GNNFXF

SEQ ID NO: 79 - protein, 7 aa, Artificial Sequence
  Synthetic Construct
LYRFVST

SEQ ID NO: 80 - protein, 4 aa, Artificial Sequence
  Synthetic Construct
PPPM

SEQ ID NO: 81 - protein, 6 aa, Artificial Sequence
  Synthetic Construct
  MISC_FEATURE (2)..(2): Xaa can be E or T
  MISC_FEATURE (3)..(3): Xaa can be F or M
  MISC_FEATURE (4)..(4): Xaa can be S, T or V
  MISC_FEATURE (6)..(6): Xaa can be K, N or T
FXXXYX

SEQ ID NO: 82 - protein, 8 aa, Artificial Sequence
  Synthetic Construct
MLRTGNNF

SEQ ID NO: 83 - protein, 10 aa, Artificial Sequence
  Synthetic Construct
FITQYSTGQV

SEQ ID NO: 84 - protein, 120 aa, Artificial Sequence
  Synthetic Construct
GTEGCLPAFPPQVFTLPQYGYATLNRDNTENPTERSSFFCLEYFPSKMLRTGNNFEMVYKFENVPFHSMYAHSQSLDRLMNPLLDQYLWELQSTTSGGTLNQGNSATNFAKLTKTNFSGY

SEQ ID NO: 85 - protein, 111 aa, Artificial Sequence
  Synthetic Construct
GTEGCLPAFPPQVFTLPQYGYATLNRDNTENPTERSSFFCLEYFPSKMLRTGNNFTFSYTFEDVPFHSSYAHSQSLDRLMNPLIDQYLYYLSKTAGGLAFSQAGPTTMRNQ

SEQ ID NO: 86 - protein, 113 aa, Artificial Sequence
  Synthetic Construct
GTEGCLPAFPPQVFTLPQYGYATLNRDNTENPTERSSFFCLEYFPSKMLRTGNNFEFTYNFEEVPFHSSFAPSQNLFKLANPLVDQYLYRFVSTDTSGAVQFKKNLAGRYANT

SEQ ID NO: 87 - protein, 117 aa, Artificial Sequence
  Synthetic Construct
AHEGCLPPFPADVFMIPQYGYLTLNDGSQAVGRSSFYCLEYFPSQMLRTGNNFQFSYEFENVPFHSSYAHSQSLDRLMNPLIDQYLYYLSRTNTPSGTTTQSRLQFSQAGASDIRDQ

SEQ ID NO: 88 - protein, 118 aa, Artificial Sequence
  Synthetic Construct
AHEGCLPPFPADVFMIPQYGYLTLNDGSQAVGRSSFYCLEYFPSQMLRTGNNFQFSYEFENVPFHSSYAHSQSLDRLMNPLIDQYLYYLNRTQGTTSGTTNQSRLLFSQAGPQSMSLQ

SEQ ID NO: 89 - protein, 118 aa, Artificial Sequence
  Synthetic Construct
AHEGCLPPFPADVFMIPQYGYLTLNDGSQAVGRSSFYCLEYFPSQMLRTGNNFQFSYEFENVPFHSMYAHSQSLDRLMNPLIDQYLWGLQSTTTGTTLNAGTATTNFTKLRPTNFSNF

SEQ ID NO: 90 - protein, 111 aa, Artificial Sequence
  Synthetic Construct
AHEGCLPPFPADVFMIPQYGYLTLNDGSQAVGRSSFYCLEYFPSQMLRTGNNFQFSYEFENVPFHSSFAPSQNLFKLANPLVDQYLYRFVSTNNTGGVQFNKNLAGRYANT

SEQ ID NO: 91 - protein, 117 aa, Artificial Sequence
  Synthetic Construct
AHEGCLPPFPADVFMIPQYGYLTLNDGSQAVGRSSFYCLEYFPSQMLRTGNNFQFSYTFEDVPFHSSYAHSQSLDRLMNPLIDQYLYYLNRTQNQSGSAQNKDLLFSRGSPAGMSVQ

SEQ ID NO: 92 - protein, 118 aa, Artificial Sequence
  Synthetic Construct
AHEGCLPPFPADVFMIPQYGYLTLNDGSQAVGRSSFYCLEYFPSQMLRTGNNFQFSYSFEDVPFHSSYAHSQSLDRLMNPLIDQYLYYLARTQSNPGGTAGNRELQFYQGGPSTMAEQ

SEQ ID NO: 93 - protein, 117 aa, Artificial Sequence
  Synthetic Construct
AHEGCLPPFPADVFMIPQYGYLTLNDGSQAVGRSSFYCLEYFPSQMLRTGNNFQFTYTFEDVPFHSSYAHSQSLDRLMNPLIDQYLYYLSRTQTTGGTANTQTLGFSQGGPNTMANQ

SEQ ID NO: 94 - protein, 118 aa, Artificial Sequence
  Synthetic Construct
AHQGCLPPFPADVFMIPQYGYLTLNNGSQAVGRSSFYCLEYFPSQMLRTGNNFEFSYQFEDVPFHSMYAHSQSLDRLMNPLIDQYLWGLQSTTTGTTLNAGTATTNFTKLRPTNFSNF

SEQ ID NO: 95 - protein, 116 aa, Artificial Sequence
  Synthetic Construct
AHQGCLPPFPADVFMIPQYGYLTLNNGSQAVGRSSFYCLEYFPSQMLRTGNNFEFSYQFEDVPFHSSYAHSQSLDRLMNPLIDQYLYYLSKTINGSGQNQQTLKFSVAGPSNMAVQ

SEQ ID NO: 96 - protein, 119 aa, Artificial Sequence
  Synthetic Construct
TNTAPTAGTYNHQEILPGSVWMDRDVYLQGPIWAKIPETGAHFHPSPAMGGFGLKYPPPMMLIKNTPVPGNITSFSDVPVSSFITQYSTGQVTVEMEWELKKENSKRWNPEIQYTNNYN

SEQ ID NO: 97 - protein, 120 aa, Artificial Sequence
  Synthetic Construct
GNRQAATADVNTQGVLPGMVWQDRDVYLQGPIWAKIPHTDGHFHPSPLMGGFGMKHPPPQILIKNTPVPADPPTAFNKDKLNSFITQYSTGQVSVEIEWELQKENSKRWNPEIQYTSNYY

SEQ ID NO: 98 - protein, 120 aa, Artificial Sequence
  Synthetic Construct
SNTAPTTRTVNDQGALPGMVWQDRDVYLQGPIWAKIPHTDGHFHPSPLMGGFGLKHPPPQILIKNTPVPADPPTAFNKDKLNSFITQYSTGQVSVEIEWELQKENSKRWNPEIQYTSNYY

SEQ ID NO: 99 - protein, 120 aa, Artificial Sequence
  Synthetic Construct
NSNLPTVDRLTALGAVPGMVWQNRDIYYQGPIWAKIPHTDGHFHPSPLIGGFGMKHPPPQILIKNTPVPADPPTAFNKDKLNSFITQYSTGQVSVEIEWELQKENSKRWNPEIQYTSNYY

SEQ ID NO: 100 - protein, 120 aa, Artificial Sequence
  Synthetic Construct
STTAPATGTYNLQEIVPGSVWMERDVYLQGPIWAKIPETGAHFHPSPAMGGFGMKHPPPQILIKNTPVPADPPTAFNKDKLNSFITQYSTGQVSVEIEWELQKENSKRWNPEIQYTSNYY

SEQ ID NO: 101 - protein, 120 aa, Artificial Sequence
  Synthetic Construct
SSTDPATGDVHVMGALPGMVWQDRDVYLQGPIWAKIPHTDGHFHPSPLMGGFGLKHPPPQILIKNTPVPADPPTAFNKDKLNSFITQYSTGQVSVEIEWELQKENSKRWNPEIQYTSNYY

SEQ ID NO: 102 - protein, 120 aa, Artificial Sequence
  Synthetic Construct
ANTAAQTQVVNNQGALPGMVWQNRDVYLQGPIWAKIPHTDGNFHPSPLMGGFGLKHPPPQILIKNTPVPADPPTAFNKDKLNSFITQYSTGQVSVEIEWELQKENSKRWNPEIQYTSNYY

SEQ ID NO: 103 - protein, 120 aa, Artificial Sequence
  Synthetic Construct
QNTAPQIGTVNSQGALPGMVWQNRDVYLQGPIWAKIPHTDGNFHPSPLMGGFGLKHPPPQILIKNTPVPADPPTAFNKDKLNSFITQYSTGQVSVEIEWELQKENSKRWNPEIQYTSNYY

SEQ ID NO: 104 - protein, 120 aa, Artificial Sequence
  Synthetic Construct
NSNLPTVDRLTALGAVPGMVWQNRDIYYQGPIWAKIPHTDGHFHPSPLIGGFGLKHPPPQILIKNTPVPADPPTTFSQAKLASFITQYSTGQVSVEIEWELQKENSKRWNPEIQYTSNYY

SEQ ID NO: 105 - protein, 120 aa, Artificial Sequence
  Synthetic Construct
AQAQAQTGWVQNQGILPGMVWQDRDVYLQGPIWAKIPHTDGNFHPSPLMGGFGMKHPPPQILIKNTPVPADPPTTFSQAKLASFITQYSTGQVSVEIEWELQKENSKRWNPEIQYTSNYY

SEQ ID NO: 106 - protein, 120 aa, Artificial Sequence
  Synthetic Construct
ATTVPTVDDVDGVGVYPGMVWQDRDIYYQGPIWAKIPHTDGHFHPSPLIGGFGLKSPPPQIFIKNTPVPANPATTFSPARINSFITQYSTGQVTVEMEWELKKENSKRWNPEIQYTNNYN

SEQ ID NO: 107 - protein, 120 aa, Artificial Sequence
  Synthetic Construct
SSTQATTAIVNAQGILPGMVWQDRDVYLQGPIWAKIPHTDGHFHPSPLMGGFGLKHPPPQILIKNTPVPSNPPEKFTQEKLNAFITQYSTGQVTVEMEWELKKENSKRWNPEIQYTNNYN

(19) United States
(12) Patent Application Publication
(10) Pub. No.: US 2025/0188126 A1
(43) Pub. Date: Jun. 12, 2025

(54) ADENO-ASSOCIATED VIRAL VECTOR CAPSIDS WITH IMPROVED TISSUE TROPISM

(71) Applicant: ALEXION PHARMA INTERNATIONAL OPERATIONS LIMITED, Dublin 15 (IE)

(72) Inventors: Seungil Han, Mystic, CT (US); Yuhang Liu, Glastonbury, CT (US); Joseph Elias Rabinowitz, Philadelphia, PA (US); Charles Shyng, Apex, NC (US)

(73) Assignee: ALEXION PHARMA INTERNATIONAL OPERATIONS LIMITED, Dublin 15 (IE)

(21) Appl. No.: 18/288,461

(22) PCT Filed: Apr. 22, 2022
(86) PCT No.: PCT/IB2022/053775

§ 371 Date: Oct. 26, 2023

Related U.S. Application Data
(60) Provisional application No. 63/179,968, filed on Apr. 26, 2021.

(51) Int. Cl. C07K 14/005; C12N 15/86

(52) U.S. Cl. CPC C07K 14/005; C12N 15/86; C12N 2750/14122; C12N 2750/14143; C12N 2750/14151

(57) ABSTRACT

The present invention provides novel chimeric AAV capsid proteins and their use in adeno-associated viral (AAV) vectors, including recombinant AAV (rAAV) vectors, and compositions thereof. The chimeric AAV capsid proteins have advantageous properties including tropism that differs from that of the parental AAV capsid.

### REFERENCE TO SEQUENCE LISTING

This application is being filed electronically via EFS-Web and includes an electronically submitted sequence listing in.txt format. The .txt file contains a sequence listing entitled “PC072719_SequenceListing_ST25.txt” created on Feb. 15, 2022 and having a size of 254 KB. The sequence listing contained in this .txt file is part of the specification and is herein incorporated by reference in its entirety.

### FIELD

The present invention relates to the field of chimeric AAV capsid proteins and their use in adeno-associated viral (AAV) vectors, including recombinant AAV (rAAV) vectors. Specifically, the present invention relates to chimeric AAV capsids with advantageous properties including tropism that differs from tropism of the parental AAV capsid.

### BACKGROUND OF THE INVENTION

Gene therapy, including those therapies that use a recombinant AAV (rAAV) vector to deliver a therapeutic transgene, has the potential to treat a wide range of serious diseases for which no cure, and in many cases, limited treatment exists (Wang et al. (2019) Nature Reviews 18:358-378). Gene therapy, using a rAAV vector, introduces a healthy copy of a defective gene to a patient which then expresses a protein with a normal structure or function.

rAAV vectors can be made using any of the naturally occurring, synthetic or chimeric serotypes of AAV. A serotype can be defined by the inability of an antibody that is reactive to a viral capsid protein of one serotype to neutralize another serotype (Choi et al. (2005) Curr. Gene Ther. 5 (3): 299-310). The lack of neutralization indicates the same serology with the newer AAV as a subgroup or variant. New AAV isolates may exhibit differences in capsid structure, antigenic diversity, and varying tissue tropism (Asokan et al. (2011) Molecular Ther. 20 (4) 699-708). Tropism for some AAV serotypes has been characterized including skeletal muscle tropism of AAV1, AAV6 and AAV9, cardiac muscle tropism of AAV1, AAV6 and AAV9, lung tropism of AAV5, liver tropism of AAV8, ocular tropism of AAV4 and AAV8, CNS tropism of AAV1, AAV5, AAV8, AAV9, AAVv66 and others (Asokan et al. (2011) Molecular Ther. 20 (4) 699-708; Hsu et al. (2020) Nat. Comm. 11:3279; Srivastava et al. (2016) Curr. Opin. Virol. 21:75-80). A chimeric capsid with a mixture of AAV1, AAV2, AAV6, AA8 and AAV9 exhibits >95% tropism for striatal oligodendrocytes (Powell et al. (2016) Gene Ther. 23:807-814). Chimeric capsids have been derived from AAV12 VP1/2 sequences and from VP3 sequences of AAV6 and demonstrate enhanced infection of human T cells, hematopoietic stem cells and neuronal cell lines (Viney et al. (2021) J. Virol. 95 (7): 1-15). AAV capsids that target specific cell types or tissues, or have a specific tropism profile across cells types or tissues, are beneficial for use in gene therapy programs.

### SUMMARY OF THE INVENTION

The present disclosure provides novel chimeric AAV capsids that exhibit advantageous properties, including a tropism profile that is different from the tropism profile of a parental AAV capsid. Furthermore, the novel chimeric AAV capsids are amenable to production under standard protocols and generate titers consistent with that of parental AAV serotypes. In another aspect, the novel chimeric AAV capsids are amenable to purification under standard protocols for generating drug substance for clinical gene therapy program.

In some aspects, the present disclosure provide an AAV capsid polypeptide comprising an amino acid sequence of a parental AAV VP1 polypeptide, comprising a substitution of amino acids from a region between β-sheet G and β-sheet H with amino acids from a region between β-sheet G and β-sheet H of an alternative AAV VP1 polypeptide.

In some embodiments, the region between β-sheet G and β-sheet H of the parental AAV VP1 polypeptide and the alternative AAV VP1 polypeptide comprises about 222 to about 235 amino acids. In some embodiments, the regions between β-sheet G and β-sheet H comprise amino acids from within β-sheet G and β-sheet H. In some embodiments, β-sheet G of the parental AAV VP1polypeptide and β-sheet G of the alternative AAV VP1 polypeptide comprises or consists of an amino acid sequence of FX₁X₂X₃YX₄, wherein X₁ is E, Q, or T; wherein X₂ is F, I, or M; wherein X₃ is S, T or V; and wherein X₄ is E, K, N, Q, S or T (SEQ ID NO: 52). In some embodiments, β-sheet H of the parental AAV VP1 polypeptide and β-sheet H of the alternative AAV VP1 polypeptide comprises or consists of an amino acid sequence of X₁X₂X₃IKNT, wherein X₁ is Q, or M; wherein X₂ is I or M; and wherein X₃ is L, M or F (SEQ ID NO: 65).

In some embodiments, a serotype of the parental AAV VP1 polypeptide is selected from the group consisting of AAV2, AAV3B, AAV4, AAV5, AAV6, AAV7, AAV8, AAV9, AAVrh10, AAVbovine, AAVporcine4 and AAVporcine5. In some embodiments, a serotype of the alternative AAV VP1 polypeptide is selected from the group consisting of AAV2, AAV3B, AAV4, AAV5, AAV6, AAV7, AAV8, AAV9, AAVrh10, AAVbovine, AAVporcine4 and AAVporcine5. In some embodiments, the amino acid sequence of the parental AAV VP1 polypeptide, of the alternative AAV VP1 polypeptide or both is set forth in any one of SEQ ID NO: 1, SEQ ID NO:3, SEQ ID NO:5, SEQ ID NO:7, SEQ ID NO:9, SEQ ID NO:11, SEQ ID NO: 13, SEQ ID NO:15, SEQ ID NO: 17, SEQ ID NO: 19, SEQ ID NO:21 and SEQ ID NO:23. In some embodiments, the amino acid sequence of the parental AAV VP1 polypeptide, of the alternative AAV VP1 polypeptide or both is encoded by a nucleic acid set forth in any one of SEQ ID NO:2, SEQ ID NO:4, SEQ ID NO:6, SEQ ID NO:8, SEQ ID NO:10, SEQ ID NO:12, SEQ ID NO:14, SEQ ID NO: 16, SEQ ID NO:18, SEQ ID NO: 20, SEQ ID NO:22 and SEQ ID NO: 24.

In some embodiments, the region between β-sheet G and β-sheet H of the parental AAV VP1 polypeptide, the region between β-sheet G and β-sheet H of the alternative AAV VP1 polypeptide or both is flanked by an amino acid sequence comprising or consisting of the sequence VPFHS, (SEQ ID NO:75) at the amino terminal end and is flanked by an amino acid sequence comprising or consisting of the sequence KHPPP (SEQ ID NO:76) or MKHPPP (SEQ ID NO: 77) at the carboxy terminal end.

In some embodiments, the serotype of the parental AAV VP1 polypeptide is AAV9 or AAVrh10 and wherein the serotype of the alternative AAV VP1 polypeptide is selected from the group consisting of AAV2, AAV3B, AAV4, AAV5 and AAV9. In some embodiments, the AAV capsid polypeptide comprises an amino acid sequence at least 90%, 95%, 98% or 99% identical to an amino acid sequence of any one of SEQ ID NO:25, SEQ ID NO:27, SEQ ID NO: 29, SEQ ID NO:31, SEQ ID NO:39, and SEQ ID NO:41. In some embodiments, AAV capsid polypeptide comprises or consists of the amino acid sequence of any one of SEQ ID NO: 25, SEQ ID NO:27, SEQ ID NO:29, SEQ ID NO:31, SEQ ID NO:39, and SEQ ID NO:41.

In some embodiments, the region between β-sheet G and β-sheet H of the parental AAV VP1 polypeptide, the region between β-sheet G and β-sheet H of the alternative AAV VP1 polypeptide or both is flanked by an amino acid sequence comprising or consisting of the sequence GNNFX₁F wherein X₁ is E, Q or T (SEQ ID NO:78) at the amino terminal end and flanked by an amino acid sequence comprising or consisting of the sequence KHPPP (SEQ ID NO: 76) at the carboxy terminal end.

In some embodiments, the serotype of the parental AAV VP1 polypeptide is AAV9 and wherein the serotype of the alternative AAV VP1 polypeptide is selected from the group consisting of AAV6, AAV7 and AAV8. In some embodiments, the AAV capsid polypeptide comprises an amino acid sequence at least 90%, 95%, 98% or 99% identical to any one of SEQ ID NO:33, SEQ ID NO:35 and SEQ ID NO:37. In some embodiments, the AAV capsid polypeptide comprises or consists of the amino acid sequence of any one of SEQ ID NO:33, SEQ ID NO:35 and SEQ ID NO:37.

In some embodiments, the region between β-sheet G and β-sheet H of the parental AAV VP1polypeptide, the region between β-sheet G and β-sheet H of the alternative AAV VP1 polypeptide or both is flanked by an amino acid sequence comprising or consisting of the sequence LYRFVST (SEQ ID NO:79) at the amino terminal end and flanked by an amino acid sequence comprising or consisting of the sequence PPPM (SEQ ID NO:80) at the carboxy terminal end. In some embodiments, the serotype of the parental AAV VP1 polypeptide is AAV5 and wherein the serotype of the alternative AAV VP1 polypeptide is AAVporcine5. In some embodiments, the AAV capsid polypeptide comprises an amino acid sequence at least 90%, 95%, 98% or 99% identical to SEQ ID NO:47. In some embodiments, the polypeptide comprises or consists of the amino acid sequence of SEQ ID NO:47.

In some aspects, the disclosure provides an AAV capsid polypeptide comprising an amino acid sequence of a parental AAV VP1 polypeptide, comprising a substitution of amino acids from a region between β-sheet G and β-sheet I with amino acids from a region between β-sheet G and β-sheet I of an alternative AAV VP1 polypeptide. In some embodiments, the regions between β-sheet G and β-sheet I comprise amino acids from within β-sheet G and β-sheet I. In some embodiments, the β-sheet G of the parental AAV VP1 polypeptide and of the alternative AAV VP1 polypeptide comprises or consists of an amino acid sequence of FX₁X₂X₃YX₄, wherein X₁ is E or T; wherein X₂ is F or M; wherein X₃ is S, T or V; and wherein X₄ is K, N or T (SEQ ID NO:81).

In some embodiments, the parental AAV VP1, the alternative AAV VP1 or both is selected from the group consisting of AAV2, AAV5, AAV6, AAV8, AAVbovine, AAVporcine4 and AAVporcine5.

In some embodiments, the β-sheet I of the parental AAV VP1 polypeptide and of the alternative AAV VP1 polypeptide comprises or consists of an amino acid sequence of TQYSTGQVX₁VX₂X₃X₄WEX₅X₆, wherein X₁ is A, S or T; wherein X₂ is E, K or Q; wherein X₃ is I or M; wherein X₄ is D or E; wherein X₅ is I or L; and wherein X₆ is Q or K (SEQ ID NO:71).

In some embodiments, the parental AAV VP1, the alternative AAV VP1 or both is selected from the group consisting of AAV2, AAV3B, AAV4, AAV5, AAV6, AAV7, AAV8, AAV9, AAVrh10, AAVbovine, AAVporcine4 and AAVporcine5. In some embodiments, the amino acid sequence of the parental AAV VP1 polypeptide, of the alternative AAV VP1 polypeptide, or both is set forth in any one of SEQ ID NO:1, SEQ ID NO:3, SEQ ID NO:7, SEQ ID NO:9, SEQ ID NO: 11, SEQ ID NO: 13, SEQ ID NO: 15, SEQ ID NO:17, SEQ ID NO:19, SEQ ID NO:21 and SEQ ID NO:23. In some embodiments, the amino acid sequence of the parental AAV VP1 polypeptide, of the alternative AAV VP1 polypeptide or both is encoded by a nucleic acid set forth in any one of SEQ ID NO:2, SEQ ID NO:4, SEQ ID NO:8, SEQ ID NO:10, SEQ ID NO:12, SEQ ID NO:14, SEQ ID NO:16, SEQ ID NO:18, SEQ ID NO: 20, SEQ ID NO:22 and SEQ ID NO: 24.

In some embodiments, the region between β-sheet G and β-sheet I of the parental AAV VP1polypeptide and the region between β-sheet G and β-sheet I of the alternative AAV VP1 polypeptide is flanked by an amino acid sequence comprising or consisting of the sequence MLRTGNNF (SEQ ID NO:82) at the amino terminal end and flanked by an amino acid sequence comprising or consisting of the sequence FITQYSTGQV (SEQ ID NO:83) at the carboxy terminal end. In some embodiments, the serotype of the parental AAV VP1 polypeptide is AAV5 and wherein the serotype of the alternative AAV VP1 polypeptide is AAVbovine or AAVporcine4. In some embodiments, the AAV capsid polypeptide comprises an amino acid sequence at least 90%, 95%, 98% or 99% identical to SEQ ID NO:43 or SEQ ID NO:45. In some embodiments, the AAV capsid polypeptide comprises or consists of the amino acid sequence of SEQ ID NO:43 or SEQ ID NO:45.

In some aspects, the disclosure provides an AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV6 VP1 polypeptide, and wherein the polypeptide comprises the amino acid sequence of SEQ ID NO:33. In some embodiments, any one or more of amino acids E547 S552, N553, A555, N558 V559 from the AAV6 VP1 polypeptide and any one or more of amino acids N710, N711 and V721 from the AAV9 VP1 polypeptide interact with the PKD1 receptor. In some embodiments, any one or more of amino acids W504 and T505 from the AAV6 VP1 polypeptide and wherein any one or more of amino acids S263, S269, S386, Q387, D384, N270 from the AAV9 VP1 polypeptide interact with the PKD2 receptor.

In some aspects, the disclosure provides an AAV capsid polypeptide comprising or consisting of an amino acid sequence selected from the group consisting of SEQ ID NO:25, SEQ ID NO:27, SEQ ID NO:29, SEQ ID NO:31, SEQ ID NO:33, SEQ ID NO:35, SEQ ID NO: 37, SEQ ID NO:39, SEQ ID NO:41, SEQ ID NO:43, SEQ ID NO:45 and SEQ ID NO:47.

In some aspects, the disclosure provides an AAV capsid polypeptide comprising or consisting of an amino acid sequence encoded by a nucleic acid sequence selected from the group consisting of SEQ ID NO:26, SEQ ID NO:28, SEQ ID NO:30, SEQ ID NO:32, SEQ ID NO: 34, SEQ ID NO:36, SEQ ID NO:38, SEQ ID NO:40, SEQ ID NO:42, SEQ ID NO:44, SEQ ID NO: 46 and SEQ ID NO:48.

In some aspects, the disclosure provides a recombinant adeno-associated viral (rAAV) vector comprising an AAV capsid polypeptide disclosed herein. In some embodiments, the rAAV vector further comprises a nucleic acid comprising a transgene. In some embodiments, the transgene encodes a therapeutic protein or a reporter protein. In some embodiments, the reporter protein is a GFP protein. In some embodiments, the tropism of the rAAV vector differs from the tropism of an otherwise identical rAAV vector comprising an AAV capsid polypeptide of a parental serotype.

In some aspects, the disclosure provides a nucleic acid encoding an AAV capsid polypeptide disclosed herein. In some embodiments, the nucleic acid is a plasmid.

In some aspects, the disclosure provides a host cell comprising a nucleic acid encoding a AAV capsid polypeptide disclosed herein. In some embodiments, a host cell comprises a nucleic acid encoding an AAV capsid polypeptide disclosed herein.

In some aspects, the disclosure provides a pharmaceutical composition comprising i) a rAAV vector comprising an AAV capsid polypeptide disclosed herein and a vector genome and ii) a pharmaceutically acceptable excipient. In some embodiments, a pharmaceutical composition comprises i) a rAAV vector disclosed herein and ii) a pharmaceutically acceptable excipient.

In some aspects, the disclosure provides a method of making a rAAV vector, the method comprising i) transfecting a host cell in culture with a plasmid comprising a nucleic acid encoding an AAV capsid polypeptide disclosed herein, and a plasmid comprising a nucleic acid encoding a therapeutic protein or a reporter protein and ii) isolating the rAAV vector from the host cell, from the culture media or both. In some embodiments, an amount of rAAV vector made by the host cell is substantially similar to an amount of rAAV vector made by an otherwise identical host cell comprising a nucleic acid encoding an AAV capsid polypeptide of a parental serotype. In some embodiments, an amount of rAAV vector made by the host cell comprising a nucleic acid encoding an AAV capsid polypeptide disclosed herein is about 80% to 140% of an amount of rAAV vector made by an otherwise identical host cell comprising a nucleic acid encoding an AAV capsid polypeptide of a parental serotype. In some embodiments, the parental serotype is AAV9. In some embodiments, the rAAV vector comprises an AAV capsid polypeptide comprising the amino acid sequence of SEQ ID NO:33, SEQ ID NO: 35 or SEQ ID NO: 37. In some embodiments, an amount of rAAV vector made by the host cell comprising a nucleic acid encoding an AAV capsid polypeptide disclosed herein is 5 to 100-fold greater than an amount of rAAV vector made by an otherwise identical host cell comprising a nucleic acid encoding an AAV capsid polypeptide of a parental serotype. In some embodiments, the parental serotype if AAV5. In some embodiments, the rAAV vector comprises an AAV capsid polypeptide comprising the amino acid sequence of SEQ ID NO:43, SEQ ID NO: 45 or SEQ ID NO: 47.

In some aspects, the disclosure provides a method of transducing a target cell, the method comprising contacting the target cell with a rAAV vector comprising an AAV capsid polypeptide disclosed herein, a rAAV vector disclosed herein or a pharmaceutical composition disclosed herein such that the rAAV vector is introduced into the target cell. In some embodiments, the target cell is a liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells, hematopoietic (e.g., blood or lymph) or stem (e.g., pluripotent or multipotent progenitor) cell. In some embodiments, the target cell is an isolated cell and transduction occurs ex vivo. In some embodiments, the target cell is a cell within an organism and transduction occurs in vivo. In some embodiments, the transduced target cell expresses a therapeutic protein or a reporter protein. In some embodiments, the reporter protein is green fluorescent protein (GFP) or enhanced green fluorescent protein (eGFP). In some embodiments, the transduced cell within an organism expresses the transgene encoding a therapeutic protein.

In some aspects, the disclosure provides a rAAV vector comprising an AAV capsid polypeptide disclosed herein, a rAAV vector disclosed herein or a pharmaceutical composition disclosed herein for use in treating and/or preventing a disease, disorder or condition. In some aspects, the disclosure provides use of a rAAV vector comprising an AAV capsid polypeptide disclosed herein, a rAAV vector disclosed herein or a pharmaceutical composition disclosed herein in the manufacture of a medicament for treating and/or preventing a disease, disorder or condition.

### BRIEF DESCRIPTION OF THE FIGURES

FIG. 1 depicts exemplary design of AAV capsids with GH loop or GI loop substitutions.

FIG. 2 depicts exemplary production values of rAAV vectors in HEK293T cells in 30 mL cultures with an AAV9 or AAV5 capsid with a GH loop or GI loop substitution from AAVbovine (AAV5GHBov), AAVporcine4 (AAV5GHpo5), AAVporcine5 (AAV5GHpo5), AAV7 (AAV9GH7) or AAV8 (AAV9GH8) as compared to production of parental rAAV9 and rAAV5 vectors. Data are expressed as viral genomes (vg)/mL.

FIG. 3 depicts exemplary 2 liter suspension culture production values of rAAV vectors with an AAV9 capsid with a GH loop substitution from AAV6 (AAV9GH6), AAV7 (AAV9GH7) or AAV8 (AAV9GH8) as compared to production of a parental rAAV9 vector. Data are expressed as viral genomes (vg) produced per producer cell.

FIG. 4 depicts exemplary whole animal and tissue biodistribution of rAAV vectors with an enhanced green fluorescent protein (eGFP) transgene and an AAV9 capsid or an AAV9 capsid with a GH loop substitution from AAV6 (AAV9GH6) or AAV7 (AAV9GH7) administered intravenously to mice. Bioluminescence was measured in whole animals and in ex vivo tissues as photons/second. Control animals administered saline as a vehicle control.

FIG. 5 depicts exemplary quantification of tissue biodistribution of rAAV vectors with an eGFP transgene and an AVV9 capsid or an AAV9 capsid with a GH loop substitution from AAV6 (AAV9GH6) or AAV7 (AAV9GH7) administered intravenously to mice. Bioluminescence was measured in whole animals and in tissues as photons/second and normalized to non-injected controls (n=2). Significant difference in relative luminescence in liver tissue is indicated by **.

FIG. 6 depicts exemplary whole animal biodistribution of rAAV vectors with an eGFP transgene and an AAV9 capsid or an AAV9 capsid with a GH loop substitution from AAV6 (GH6), AAV7 (GH7) or AAV8 (GH8) administered by ICV to mice. Bioluminescence was measured in whole animals as photons/second. Control animals administered saline as a vehicle control.

FIG. 7 depicts exemplary ex vivo tissue biodistribution of rAAV vectors with an eGFP transgene and an AAV9 capsid or an AAV9 capsid with a GH loop substitution from AAV7 (GH7) or AAV8 (GH8) administered by ICV to mice. Bioluminescence was measured in tissues as photons/second. Animals administered saline as a vehicle control.

FIG. 8 depicts biodistribution of rAAV vectors by dd PCR in tissues from C57/BI6 mice administered 3E+13 vg/kg or 5E+12 vg/kg (n=4) of rAAV vector with an eGFP transgene and an AAV9 capsid or an AAV9 capsid with a GH loop substitution from AAV6 (AAV9GH6), AAV7 (AAV9GH7) or AAV8 (AAV9GH8). Significant differences in biodistribution are indicated by ****.

FIG. 9 depicts exemplary RNA expression levels in liver tissue from mice administered intravenously 3E+13 vg/kg rAAV vectors with an eGFP transgene and an AAV9 capsid or an AAV9 capsid with a GH loop substitution from AAV6 (AAV9GH6), AAV7 (AAV9GH7) or AAV8 (AAV9GH8).

FIG. 10 depicts exemplary IHC staining in liver and heart tissue of mice administered 3E+13 vg/kg of rAAV vector with an eGFP transgene and a AAV9 capsid or a AAV capsid with a GH loop substitution from AAV6 (AAV9GH6), AAV7 (AAV9GH7) or AAV8 (AAV9GH8).

FIG. 11 depicts exemplary quantification of IHC GFP positive areas of whole heart (top left panel), ventricle (top right panel) and liver (bottom panel) tissue from mice administered a rAAV vector with an eGFP transgene and a AAV9 capsid or a AAV capsid with a GH loop substitution from AAV6 (AAV9GH6) or AAV7 (AAV9GH7). Each point represents a single animal. Bars represent mean+/−SD. Significance determined by one-way ANOVA with Tukey's multiple comparisons test. ns=not significant; * p<0.05, ** p<0.01, **** p<0.0001.

FIG. 12A-12C depicts exemplary structures of an AAV-GH-AAV6 capsid by cryoEM analysis. FIG. 12A depicts the overall EM density map of the AAV9-GH-AAV6 capsid. FIG. 12B depicts the atomic structure model of one capsomer fitted into the density map. FIG. 12C depicts the capsid surface component from the AAV6 sequence shown in black (within the triangle) and the component from the AAV9 sequence shown in grey.

FIG. 13A-13C depicts exemplary AAV9-GH-AAV6 capsid structure comparison to AAV9 capsid structure. FIG. 13A depicts the AAV9-GH-AAV6 capsomer structure overlaid (based on alignment) with the AAV9 structure (PDB code: 3UX1). The loop IV (FIG. 13B) and VIII (FIG. 13C) regions are enlarged to show the structural differences between the capsids.

FIG. 14A-14C depicts exemplary AAV9-GH-AAV6 capsid structure comparison to AAV6 capsid structure. FIG. 14A depicts the AAV9-GH-AAV6 capsomer structure overlaid (based on alignment) with the AAV6 structure (PDB code: 3SHM). The loop IV (FIG. 14B) and VIII (FIG. 14C) regions are enlarged to show the structural differences between the capsids.

FIG. 15A-15B depicts exemplary AAV receptor PKD1 binding. FIG. 15A depicts the binding regions of AAV5 capsid for the AAV receptor PKD1 with the interacting residues from AAV5 shown as spheres and labeled (Zhang et al. (2019) Nat. Comm. 10:3760). FIG. 15B depicts the predicted binding regions of an AAV9-GH-AAV6 capsid for the AAV receptor PKD1 with the interacting residues from AAV6 and AAV9 shown as spheres and labeled.

FIG. 16A-FIG. 16B depicts exemplary AAV receptor PKD2 binding. FIG. 16A depicts the binding region of AAV1 capsid for the AAV receptor PKD2 with the interacting residues from AAV1 shown as spheres and labeled (Zhang et al. (2019) Nat. Comm. 10:3760). FIG. 16B depicts the predicted binding regions of an AAV9-GH-AAV6 capsid for the AAV receptor PKD2 with the interacting residues from AAV6 and AAV9 shown as spheres and labeled.

### DETAILED DESCRIPTION OF THE INVENTION

The present disclosure provides novel chimeric AAV capsids for use in the production of rAAV vectors for gene therapy. The novel chimeric capsids comprise a parental AAV capsid serotype with a substitution of a region between the β-sheet G domain and the β-sheet H domain, or the β-sheet I domain, from an alternative AAV capsid serotype. The novel chimeric AAV capsids of the disclosure exhibit advantageous properties, including, for example, a tropism profile differing from the tropism profile of the parental AAV capsid. Furthermore, the novel chimeric AAV capsids are amenable to production under standard cell culture protocols and generate titers consistent with that of parental AAV serotypes. Furthermore, the novel chimeric AAV capsids are amenable to purification under standard protocols used to generate drug substance for clinical gene therapy program.

One of skill in the art would recognize that development of novel AAV capsids, such as those disclosed herein, and that target specific cell types or tissues, or have a specific tropism profile across cells types or tissues, would be beneficial for use in clinical gene therapy programs. Novel AAV capsids with tissue tropism that corresponds to tissues that are effected by a particular disease or condition would be particularly beneficial.

### I. Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. Numeric ranges are inclusive of the numbers defining the ranges. The terms “comprising,” “comprise,” “comprises,” “including” and “having” are intended to be inclusive and mean that there may be additional elements other than the listed elements. It is understood that wherever embodiments are described herein with the language “comprising,” otherwise analogous embodiments described in terms of “consisting of” and/or “consisting essentially of” are also provided. The following terms have the meanings given:

As used herein, the term “about,” or “approximately” is used to indicate that a value includes the standard deviation of error for the device or method being employed to determine the value. In some embodiments, the term “about” can be added to any numeral recited herein to the extent the numeral would have a standard deviation of error when measuring.

As used herein, the term “and/or” refers to and encompasses any and all possible combinations of one or more of the associated listed items, as well as the lack of combinations when interpreted in the alternative (“or”).

As used herein, the term “formulation,” “pharmaceutical formulation,” or “pharmaceutical composition” as they relate to a rAAV vector are meant to describe the rAAV vector in combination with a pharmaceutically acceptable excipient comprising, for example, a buffer, a salt, a cryoprotectant, a surfactant, and, wherein the pH is defined. A “pharmaceutical formulation” or “pharmaceutical composition” is a preparation which in in such form as to permit the biological activity of the active ingredients to be effective.

rAAV vectors are referred to as “full,” a “full capsid,” a “full vector” or a “fully packaged vector” when the capsid contains a complete vector genome, including a transgene. During production of rAAV vectors by host cells, vectors may be produced that have less packaged nucleic acid than the full capsids and contain, for example a partial or truncated vector genome. These vectors are referred to as “intermediates,” an “intermediate capsid,” a “partial” or a “partially packaged vector.” An intermediate capsid may also be a capsid with an intermediate sedimentation rate, that is a sedimentation rate between that of full capsids and empty capsids, when analyzed by analytical ultracentrifugation. Host cells may also produce viral capsids that do not contain any detectable nucleic acid material. These capsids are referred to as “empty(s),” or “empty capsids.” Full capsids may be distinguished from empty capsids based on A260/A280 ratios determined by SEC-HPLC, whereby the A260/A280 ratios have been previously calibrated against capsids (i.e., full, intermediate and empty) analyzed by analytical ultracentrifugation. Other methods known in the art for the characterization of capsids include CryoTEM, capillary isoelectric focusing and charge detection mass spectrometry. Calculated isoelectric points of ˜6.2 and ˜5.8 for empty and full AAV9 capsids, respectively have been reported (Venkatakrishnan et al., (2013) J. Virology 87.9:4974-4984).

As used herein, the term “gene” refers to a polynucleotide containing at least one open reading frame that is capable of encoding a particular polypeptide or protein after being transcribed and translated. “Gene transfer” or “gene delivery” refers to methods or systems for reliably inserting foreign DNA into host cells. Such methods can result in transient expression of non-integrated transferred DNA, extrachromosomal replication and expression of transferred replicons (e.g. episomes), and/or integration of transferred genetic material into the genomic DNA of host cells.

As used herein, the terms “host cell,” refers to a cell into which an exogenous nucleic acid has been introduced and includes the progeny of such a cell. A host cell includes a “transfectant,” “transformant,” “transformed cell,” and “transduced cell,” which includes the primary transfected, transformed or transduced cell, and progeny derived therefrom, without regard to the number of passages. In some embodiments, a host cell is a packaging cell for production of an rAAV vector. In some embodiments, a host cell is a “host cell line,” or “host cell culture” including it progeny derived therefrom.

As used herein, the term “identity” or “identical to” refers to the overall relatedness between polymeric molecules, e.g., between nucleic acid molecules (e.g., DNA molecules and/or RNA molecules) and/or between polypeptide molecules. “Identity” measures the percent of identical matches between two or more sequences with gap alignments addressed by a particular mathematical model of computer programs (i.e. “algorithms”).

In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98% or 99% identical.

Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, sequences can be aligned using the methods and computer programs, including BLAST, available over the world wide web at ncbi.nlm.nih.gov/BLAST/. Other alignment programs include MegAlign® program in the Lasergene® suite of bioinformatics software (DNASTAR®, Inc., Madison, WI). Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

Also, of interest is the BestFit program using the local homology algorithm of Smith and Waterman (1981, Advances in Applied Mathematics 2:482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA. Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc.

As used herein, the terms “inverted terminal repeat, “ITR,” “terminal repeat,” and “TR” refer to palindromic terminal repeat sequences at or near the ends of the AAV virus genome, comprising mostly complementary, symmetrically arranged sequences. These ITRs can fold over to form T-shaped hairpin structures that function as primers during initiation of DNA replication. They are also needed for viral genome integration into host genome, for the rescue from the host genome; and for the encapsidation of viral nucleic acid into mature virions. The ITRs are required in cis for vector genome replication and its packaging into viral particles. “5′ ITR” refers to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa. In some embodiments, at least one ITR is present at the 5′ and/or 3′ end of a recombinant vector genome such that the vector genome can be packaged into a capsid to produce a rAAV vector (also referred to herein as “rAAV vector particle” or “rAAV viral particle”) comprising the vector genome.

As used here, the term “nucleic acid construct,” refers to a non-naturally occurring nucleic acid molecule resulting from the use of recombinant DNA technology (e.g., a recombinant nucleic acid). A nucleic acid construct is a nucleic acid molecule, either single or double stranded, which has been modified to contain segments of nucleic acid sequences, which are combined and arranged in a manner not found in nature. A nucleic acid construct may be a “vector” (e.g., a plasmid, a rAAV vector genome, an expression vector, etc.), that is, a nucleic acid molecule designed to deliver exogenously created DNA into a host cell.

As used herein, the term “pharmaceutically acceptable” and “physiologically acceptable” refers to a biologically acceptable formulation, gaseous, liquid or solid, or mixture thereof, which is suitable for one or more routes of administration, in vivo delivery or contact. “Pharmaceutically acceptable excipients” (vehicles, additives) are those, which can safely be administered to a subject to provide an effective dose of the active ingredient employed. The term “excipient” or “carrier” as used herein refers to an inert substance, which is commonly used as a diluent, vehicle, preservative, binder or stabilizing agent for drugs. As used herein, the term “diluent” refers to a pharmaceutically acceptable (safe and non-toxic for administration to a human) solvent and is useful for the preparation of the liquid formulations herein. Exemplary diluents include, but are not limited to, sterile water and bacteriostatic water for injection (BWFI).

As used herein, the term “polynucleotide” or “nucleic acid” refers to a polymeric form of nucleotides, either ribonucleotides or deoxynucleotides, or a modified form of either type of nucleotide, and may be single or double stranded forms. A “polynucleotide” or a “nucleic acid” sequence encompasses its complement unless otherwise specified. As used herein, the term “isolated polynucleotide,” “isolated nucleic acid” or “isolated recombinant nucleic acid” means a polynucleotide of genomic, cDNA, or synthetic origin, or some combination thereof, which by virtue of its origin or source of derivation, has one to three of the following: (1) is not associated with all or a portion of a polynucleotide with which the “isolated polynucleotide” is found in nature, (2) is operably linked to a polynucleotide to which it is not linked in nature, or (3) does not occur in nature as part of a larger sequence.

As used herein, the term “recombinant,” refers to a vector, polynucleotide (e.g., a recombinant nucleic acid), polypeptide or cell that is the product of various combinations of cloning, restriction or ligation steps (e.g., relating to a polynucleotide or polypeptide comprised therein), and/or other procedure that results in a construct that is distinct from a product found in nature. A recombinant virus or vector (e.g., rAAV vector) comprises a vector genome comprising a recombinant nucleic acid (e.g., a nucleic acid comprising a transgene and one or more regulatory elements for the expression of the transgene). The terms respectively include replicates of the original polynucleotide construct and progeny of the original virus construct.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered. In some embodiments, a subject is a human patient with a genetic disease.

As used herein, the term “substantial” or “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve an absolute result. The term “substantial” or “substantially” therefore captures the potential lack of completeness inherent in many biological and chemical phenomena.

As used herein, the term “therapeutic protein” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a target cell, is an enzyme expressed from a transgene (i.e., an exogenous nucleic acid that has been introduced into the target cell).

As used herein, the term “transfection” refers to transfer of a recombinant nucleic acid (e.g., an expression plasmid) into a cell (e.g., a host cell) without use of a viral vector. A cell into which a recombinant nucleic acid has been introduced is referred to as a “transfected cell.” A transfected cell may be a host cell (e.g., a CHO cell, Pro10 cell, HEK293 cell) comprising an expression plasmid/vector for producing a recombinant AAV vector. In some embodiments, a transfected cell (e.g., a packing cell) may comprise a plasmid comprising a transgene (e.g., a transgene encoding a therapeutic protein), a plasmid comprising an AAV rep gene and an AAV cap gene (e.g., a AAV cap gene with a GH loop substitution of a GI loop substitution) and a plasmid comprising a helper gene. Many transfection techniques are known in the art, which include, but are not limited to, electroporation, calcium phosphate precipitation, microinjection, cationic or anionic liposomes, and liposomes in combination with a nuclear localization signal.

As used herein, the term “transduction” refers to transfer of a nucleic acid (e.g., a vector genome) by a viral vector (e.g., rAAV vector) to a cell (e.g., a target cell, including, but not limited to, a cell within a mammal). In some embodiments, a gene therapy for the treatment disease includes transducing a vector genome comprising a modified nucleic acid encoding a therapeutic protein into a target cell. A cell into which a transgene has been introduced by a virus or a viral vector is referred to as a “transduced cell.” In some embodiments, a transduced cell is an isolated cell and transduction occurs ex vivo. In some embodiments, a transduced cell is a cell within an organism (e.g., a subject) and transduction occurs in vivo. A transduced cell may be a target cell of an organism which has been transduced by a recombinant AAV vector such that the target cell of the organism expresses a polynucleotide (e.g., a transgene encoding a therapeutic protein).

A cell that may be transduced includes a cell of any tissue or organ type, or any origin (e.g., mesoderm, ectoderm or endoderm). Non-limiting examples of cells include liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells or hematopoietic (e.g., blood or lymph) cells. Additional examples include stem cells, such as pluripotent or multipotent progenitor cells that develop or differentiate into liver (e.g., hepatocytes, sinusoidal endothelial cells), pancreas (e.g., beta islet cells, exocrine cells), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblast, myocytes), synoviocytes, chondrocytes, osteoclasts, epithelial cells, endothelial cells, salivary gland cells, inner ear nervous cells or hematopoietic (e.g., blood or lymph) cells.

In some embodiments, cells present within particular areas of a tissue or organ (e.g., liver) may be transduced by an rAAV vector (e.g., an rAAV comprising a therapeutic transgene, a reporter transgene) that is administered to the tissue or organ.

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector). A transgene may be operably linked to a control sequence, such as a promoter. It will be appreciated by those of skill in the art that expression control sequences can be selected based on an ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. An example of a transgene is a nucleic acid encoding a therapeutic polypeptide.

As used herein, the term “vector” refers to a plasmid, virus (e.g., a rAAV), cosmid, or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments, a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) signal sequence and/or an ITR. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence (e.g., a transgene). In some embodiments, when delivered to a host cell, the nucleic acid sequence, or a portion of the nucleic acid sequence is packaged into a capsid (e.g., a chimeric capsid comprising a GH loop substitution or a GI loop substitution). A host cell may be an isolated cell or a cell within a host organism. In addition to a nucleic acid sequence (e.g., transgene) which encodes a polypeptide or protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene. Plasmid vectors may be referred to herein as “expression vectors.”

As used herein, the term “vector genome” refers to a nucleic acid that that may, but need not, be packaged/encapsidated in an AAV capsid to form a rAAV vector. Typically, a vector genome includes a heterologous polynucleotide sequence (e.g., a transgene, regulatory elements, etc.) and at least one ITR. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is referred to as the “plasmid backbone,” which is important for cloning, selection and amplification of the plasmid, a process that is needed for propagation of recombinant viral vector production, but which is not itself packaged or encapsidated into a rAAV vector. Typically, the heterologous sequence to be packaged into the capsid is flanked by the ITRs such that when cleaved from the plasmid backbone, the heterologous sequence is packaged into the capsid.

As used herein, the term “viral vector” generally refers to a viral particle that functions as a nucleic acid delivery vehicle and which comprises a vector genome (e.g., comprising a transgene which has replaced the wild type rep and cap) packaged within the viral particle (i.e., capsid) and includes, for example, lenti- and parvo-viruses, including AAV serotypes and variants (e.g., rAAV vectors). As noted elsewhere herein, a recombinant viral vector does not comprise a virus genome with a rep and/or a cap gene; rather, these sequences have been removed to provide capacity for the vector genome to carry a transgene of interest.

Unless otherwise defined, all technical and scientific terms used herein have the same meaning as commonly understood by one of ordinary skill in the art to which this invention belongs.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

The present disclosure provides novel chimeric AAV capsids for use in the production of rAAV vectors for gene therapy. The novel chimeric capsids comprise a parental AAV capsid serotype with a substitution of a region between the β-sheet G domain and the β-sheet H domain, or I domain, from an alternative AAV capsid serotype. Each of these aspects of the disclosure is discussed further in the ensuing sections.

### II. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### III. AAV and rAAV

### A. AAV

Adeno-associated virus is a 20-25 nm diameter non-enveloped single stranded DNA containing virus in the Dependovirus genus in the Parvoviridae family. As used herein the term “adeno-associated virus” and/or “AAV” refers to these parvoviruses and variants thereof. The term covers all subtypes and both naturally occurring and recombinant forms, except where required otherwise. AAV is ubiquitously prevalent in the human population but has not been associated with disease. Helper virus infection or DNA damaging stress will trigger latent-AAV proviruses to become active leading to viral replication.

Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome, for example at a site on human chromosome 19. Site-specific integration, as opposed to random integration, is believed to likely result in a predictable long-term expression profile. The insertion site of AAV into the human genome is referred to as AAVS1. Once introduced into a cell, polypeptides encoded by the nucleic acid can be expressed by the cell. Because AAV is not associated with any pathogenic disease in humans, a nucleic acid delivered by AAV can be used to express a therapeutic polypeptide for the treatment of a disease, disorder and/or condition in a human subject.

The canonical AAV wild-type genome comprises 4681 bases (Berns et al. (1987) Advances in Virus Research 32:243-307) and includes terminal repeat sequences (e.g., inverted terminal repeats (ITRs)) at each end which function in cis as origins of DNA replication and as packaging signals for the virus. The genome includes two large open reading frames, known as AAV replication (“AAV rep” or “rep”) and capsid (“AAV cap” or “cap”) genes, respectively. AAV rep and cap may also be referred to herein as AAV “packaging genes.” These genes code for the viral proteins involved in replication and packaging of the viral genome.

Wild type AAV comprises a small (20-25 nm) icosahedral virus capsid composed of three proteins, VP1, VP2 and VP3, with 60 capsid proteins comprising the capsid. Each viral particle has a 2—, 3- and 5-fold axis of symmetry. The three capsid genes VP1, VP2 and VP3 overlap each other within a single open reading frame and alternative splicing leads to production of VP1, VP2 and VP3 (Grieger et al. (2005) J. Virol. 79 (15): 9933-9944.). VP3 makes up to 80-90% of total subunits. VP1 has essential functions including phospholipase activity and a nuclear localization signal.

A single P40 promoter allows all three capsid proteins to be expressed at a ratio of about 1:1:10 for VP1, VP2, VP3, respectively, which complements AAV capsid production. More specifically, VP1 is the full-length protein, with VP2 and VP3 being increasingly shortened due to increasing truncation of the N-terminus. A well-known example is the capsid of AAV9 as described in U.S. Pat. No. 7,906,111, wherein VP1 comprises amino acid residues 1 to 736 of a sequence identified as number 123, VP2 comprises amino acid residues 138 to 736 of a sequence identified as number 123, and VP3 comprises amino acid residues 203 to 736 of a sequence identified as number 123. The AAV2 capsid protein sequences are available in Genbank: VP1 (735 aa; Genbank Accession No. AAC03780), VP2 (598 aa; Genbank Accession No. AAC03778) and VP3 (533 aa; Genbank Accession No. AAC03779). As used herein, the term “AAV Cap” or “cap” refers to AAV capsid proteins VP1, VP2 and/or VP3, and variants and analogs thereof.

A second open reading frame of the capsid gene encodes an assembly factor, called assembly-activating protein (AAP), which is essential for the capsid assembly process (Sonntag et al. (2011) J. Virol. 85 (23): 12686-12697).

At least four viral proteins are synthesized from the AAV rep gene-Rep 78, Rep 68, Rep 52 and Rep 40-named according to their apparent molecular weights. As used herein, “AAV rep” or “rep” means AAV replication proteins Rep 78, Rep 68, Rep 52 and/or Rep 40, as well as variants and analogs thereof. As used herein, rep and cap refer to both wild type and recombinant (e.g., modified chimeric, and the like) rep and cap genes as well as the polypeptides they encode. In some embodiments, a nucleic acid encoding a rep will comprise nucleotides from more than one AAV serotype. For instance, a nucleic acid encoding a rep protein may comprise nucleotides from an AAV2 serotype and nucleotides from an AAV3 serotype (Rabinowitz et al. (2002) J. Virology 76 (2): 791-801).

Multiple serotypes of AAV exist in nature with at least fifteen wild type serotypes having been identified from humans thus far (i.e., AAV1-AAV15). Over 150 unique AAV serotypes have been identified. Naturally occurring and variant serotypes are distinguished by having a protein capsid that is serologically distinct from other AAV serotypes. Naturally occurring and non-naturally occurring AAV serotypes include: AAV type 1 (AAV1), AAV type 2 (AAV2), AAV type 3 (AAV3) including AAV type 3A (AAV3A) and AAV type 3B (AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 12 (AAV12), AAVrh8, AAVrh10, AAVrh39, AAVrh43, AAVrh74 (see WO 2016/210170), AAVrh32.22, AAV1.1, AAV2.5, AAV6.1, AAV6.2, AAV6.3.1, AAV9.45, AAVShH10, HSC15/17, RHM4-1 (SEQ ID NO:5 of WO 2015/013313), RHM15-1, RHM15-2, RHM15-3/RHM15-5, RHM15-4, RHM15-6, AAVhu.26, AAV2i8, AAV29G, AAV2,8G9, AAV-LK03, AAV2-TT, AAV2-TT-S312N, AAV3B-S312N, avian AAV, bovine AAV, canine AAV, equine AAV, primate AAV, non-primate AAV, and ovine AAV, NP4, NP22, NP66, AAVDJ, AAVDJ/8, AAVDJ/9, among many others (see, e.g., Fields et al., “Virology”, volume 2, chapter 69 (4^{th} ed., Lippincott-Raven Publishers); U.S. Pat. No. 7,906,111; Gao et al. (2004) J. Virol. 78:6381; Morris et al. (2004) Virol. 33:375; WO 2013/063379; WO 2014/194132; WO 2015/121501; WO 2015/013313, all of which are hereby incorporated by reference). AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15 (Smith et al. (2014) Molecular Therapy 22 (9): 1625-1634, which is hereby incorporated by reference). Naturally occurring AAVs isolated from human tissues by long-read sequencing include AAVv66 with tropism for the CNS as well as AAVv33, AAVv37, AAVv40, AAVv67, AAVv70, AAVv72, AAVv84, AAVv86, AAVv87 and AAVv90 (Hsu et al. (2020) Nat. Comm. 11:3279).

Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, e.g., AAV, as well as viruses, e.g., AAV, that are not serologically distinct but that may be within a subgroup or a variant of a given serotype.

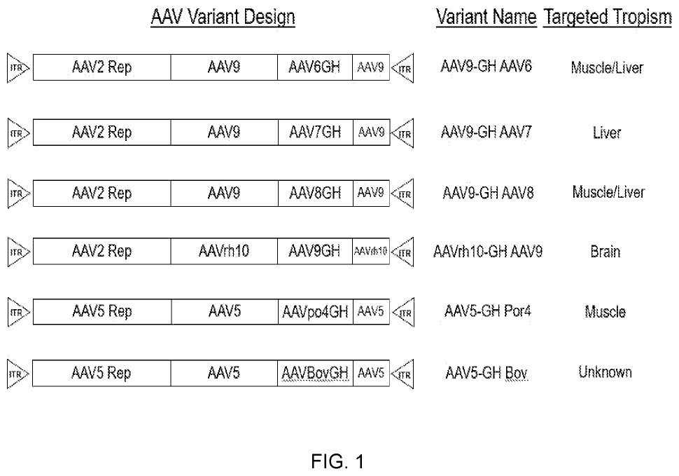

A comprehensive list and alignment of amino acid sequences of capsids of known AAV serotypes is provided by Marsic et al. (2014) Molecular Therapy 22 (11): 1900-1909, especially at supplementary FIG. 1; the entire publication is hereby incorporated by reference.

Genomic sequences of various serotypes of AAV, as well as sequences of the native inverted terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV4), U89790 (AAV4), NC_006152 (AAV5), AF028704 (AAV6), AF513851 (AAV7), AF513852 (AAV8), NC_006261 (AAV8), AY530579 (AAV9), AY631965 (AAV10), AY631966 (AAV11), and DQ813647 (AAV12); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99:11854; Moris et al. (2004) Virology 33:375-383; International Patent Publications WO 00/28061, WO 99/61601, WO 98/11244; WO 2013/063379; WO 2014/194132; WO 2015/121501, and U.S. Pat. Nos. 6,156,303 and 7,906,111, all of which are hereby incorporated by reference.

### B. Chimeric AVV

In some embodiments, a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a chimeric capsid sequence is derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Viney et al. (2021) J. Virol. 95:1-15; Choi et al. (2005) Curr. Gene Ther. 5 (3): 299-310).

The VP1 polypeptide of an AAV capsid comprises variable regions (e.g., VR I-VR IX) and β-sheet regions (e.g., A though I). The amino acid sequence between β-sheet G and β-sheet H (also referred to herein as the “GH loop”), encompasses variable region IV through variable region VIII and contains the highest level of diversity among AAV serotypes as well as among all Parvoviruses. The GH loop is at the 3-fold axis of symmetry, constitutes about 30% of the capsid and interacts with primary glycan attachment receptor. The region within the GH loop comprises about 222 to about 235 amino acids. The region within and including the GH loop comprises about 235 to about 248 amino acids.

The AAV β-sheet G has been defined as FTFSYT (SEQ ID NO:49) for AAV2, as FEITYS (SEQ ID NO:50) for AAV4 and FQFTYT (SEQ ID NO:51) for AAV8 (Nam et al. (2007) J. Virology 81 (22): 12260-12271). In some embodiments, an AAV capsid polypeptide comprises a β-sheet G comprising or consisting of the amino acid sequence of FX₁X₂X₃YX₄, wherein X₁ is E, Q, or T; wherein X₂ is F, I, or M; wherein X₃ is S, T or V; and X₄ is E, K, N, Q, S or T (SEQ ID NO: 52), and optionally wherein the AAV is serotype AAV2, AAV3B, AAV4, AAV5, AAV6, AAV7, AAV8, AAV9, AAVrh10, AAVbovine, AAVporcine4 and/or AAVporcine5.

In some embodiments, an AAV capsid polypeptide comprises a β-sheet G comprising or consisting of the amino acid sequence of FX₁X₂X₃YX₄, wherein X₁ is E, Q, or T; wherein X₂ is F or I; wherein X₃ is S or T; and X₄ is E, N, Q, S or T (SEQ ID NO:53), and optionally wherein the AAV is serotype AAV2, AAV3B, AAV4, AAV5, AAV9, AAVrh10 and/or AAVporcine5.

In some embodiments, an AAV capsid polypeptide comprises a β-sheet G comprising or consisting of the amino acid sequence of FTFSYT (SEQ ID NO:49), optionally wherein the AAV is serotype AAV2. In some embodiments, an AAV capsid polypeptide comprises a β-sheet G comprising or consisting of the amino acid sequence of FQFSYT (SEQ ID NO: 54), optionally wherein the AAV is serotype AAV3B. In some embodiments, an AAV capsid polypeptide comprises a β-sheet G comprising or consisting of the amino acid sequence of FEITYS (SEQ ID NO:50), optionally wherein the AAV is serotype AAV4. In some embodiments, an AAV capsid polypeptide comprises a β-sheet G comprising or consisting of the amino acid sequence of FEFTYN (SEQ ID NO:55), optionally wherein the AAV is serotype AAV5. In some embodiments, an AAV capsid comprises a β-sheet G comprising or consisting of the amino acid sequence of FTFSYT (SEQ ID NO:56), optionally wherein the AAV is serotype AAV6. In some embodiments, an AAV capsid polypeptide comprises a β-sheet G comprising or consisting of the amino acid sequence of FEFSYS (SEQ ID NO:57), optionally wherein the AAV is serotype AAV7. In some embodiments, an AAV capsid polypeptide comprises a β-sheet G comprising or consisting of the amino acid sequence of FQFTYT (SEQ ID NO: 51), optionally wherein the AAV is serotype AAV8. In some embodiments, an AAV capsid polypeptide comprises a β-sheet G comprising or consisting of the amino acid sequence of FQFSYE (SEQ ID NO:58), optionally wherein the AAV is serotype AAV9. In some embodiments, an AAV capsid polypeptide comprises a β-sheet G comprising or consisting of the amino acid sequence of FEFSYQ (SEQ ID NO:59), optionally wherein the AAV is serotype AAVrh10. In some embodiments, an AAV capsid polypeptide comprises a β-sheet G comprising or consisting of the amino acid sequence of FEMVYK (SEQ ID NO:60), optionally wherein the AAV is serotype AAVbovine. In some embodiments, an AAV capsid polypeptide comprises a β-sheet G comprising or consisting of the amino acid sequence of FTFSYT (SEQ ID NO: 61), optionally wherein the AAV is serotype AAVporcine4. In some embodiments, an AAV capsid polypeptide comprises a β-sheet G comprising or consisting of the amino acid sequence of FEFTYS (SEQ ID NO:62), optionally wherein the AAV is serotype AAVporcine5.

The AAV β-sheet H has been defined as QILIKNT (SEQ ID NO:63) for AAV2 and AAV8 and as QIFIKNT (SEQ ID NO:64) for AAV4 (Nam et al. (2007) J. Virology 81 (22): 12260-12271).

In some embodiments, an AAV capsid polypeptide comprises a β-sheet H comprising or consisting of the amino acid sequence of X₁X₂X₃IKNT, wherein X₁ is Q, or M; wherein X₂ is I or M; and wherein X₃ is L, M or F (SEQ ID NO:65), and optionally wherein the AAV is serotype AAV2, AAV3B, AAV4, AAV5, AAV6, AAV7, AAV8, AAV9, AAVrh10, bovine, porcine4 and/or porcine5.

In some embodiments, an AAV capsid polypeptide comprises a β-sheet H comprising or consisting of the amino acid sequence of QX₁X₂IKNT, wherein X₁ is I or M and wherein X₂ is F, L or M, (SEQ ID NO:66) and optionally wherein the AAV is serotype AAV2, AAV3B, AAV4, AAV5, AAV9, AAVrh10, bovine and/or AAVporcine4.

In some embodiments, an AAV capsid polypeptide comprises a β-sheet H comprising or consisting of the amino acid sequence of QILIKNT (SEQ ID NO:63), optionally wherein the AAV is serotype AAV2, AAV6, AAV7, AAV8, AAV9, AAVrh10, and/or AAVporcine4. In some embodiments, an AAV capsid polypeptide comprises a β-sheet H comprising or consisting of the sequence of QIMIKNT (SEQ ID NO:67), optionally wherein the AAV is serotype AAV3B. In some embodiments, an AAV capsid polypeptide comprises a β-sheet H comprising or consisting of the amino acid sequence of QIFIKNT (SEQ ID NO:64), optionally wherein the AAV is serotype AAV4 and/or AAVbovine. In some embodiments, an AAV capsid polypeptide comprises a β-sheet H comprising or consisting of the amino acid sequence of MMLIKNT (SEQ ID NO:68), optionally wherein the AAV is serotype AAV5 and/or AAVporcine5.

The AAV β-sheet I has been defined as TQYSTGQVSVEIEWELQ (SEQ ID NO:69) for AAV2 and AAV8 and as TQYSTGQVSVQIDWEIQ (SEQ ID NO:70) for AAV4 (Nam et al. (2007) J. Virology 81 (22): 12260-12271).

In some embodiments, an AAV capsid polypeptide comprises a β-sheet I comprising or consisting of the amino acid sequence of TQYSTGQVX₁VX₂X₃X₄WEX₅X₆, wherein X₁ is A, S or T; wherein X₂ is E, K or Q; wherein X₃ is I or M; wherein X₄ is D or E; wherein X₅ is I or L; and wherein X₆ is Q or K (SEQ ID NO:71), and optionally wherein the AAV is serotype AAV2, AAV3B, AAV4, AAV5, AAV6, AAV7, AAV8, AAV9, AAVrh10, bovine, porcine4 and/or porcine5.

In some embodiments, an AAV capsid polypeptide comprises a β-sheet I comprising or consisting of the amino acid sequence of TQYSTGQVX₁VX₂X₃EWEX₄X₅, wherein X₁ is A, S or T; wherein X₂ is E or K; wherein X₃ is I or M; wherein X₄ is I or L; and wherein X₅ is Q or K (SEQ ID NO:72), and optionally wherein the AAV is serotype AAV5, bovine and/or porcine4.

In some embodiments, an AAV capsid polypeptide comprises a β-sheet I comprising or consisting of the amino acid sequence of TQYSTGQVSVEIEWELQ (SEQ ID NO:69), and optionally wherein the AAV is serotype AAV2, AAV3B, AAV6, AV7, AAV8, AAV9, AAVrh10 and/or porcin4.

In some embodiments, an AAV capsid polypeptide comprises a β-sheet I comprising or consisting of the amino acid sequence of TQYSTGQVSVQIDWEIQ (SEQ ID NO:70), and optionally wherein the AAV is serotype AAV4.

In some embodiments, an AAV capsid polypeptide comprises a β-sheet I comprising or consisting of the amino acid sequence of TQYSTGQVTVEMEWELK (SEQ ID NO:73), and optionally wherein the AAV is serotype AAV5 or AAVporcine5.

In some embodiments, an AAV capsid polypeptide comprises a β-sheet I comprising or consisting of the amino acid sequence of TQYSTGQVAVKIEWEIQ (SEQ ID NO:74), and optionally wherein the AAV is serotype AAVbovine.

In some embodiments, a chimeric AAV capsid polypeptide of the disclosure comprises an amino acid sequence of a parental AAV VP1 polypeptide, comprising a substitution of amino acids from a region between β-sheet G and β-sheet H with amino acids from a region between β-sheet G and β-sheet H of an alternative AAV VP1 polypeptide. In some embodiments, the substitution includes amino acids from the β-sheet G and/or β-sheet H of either the parental AAV VP1 polypeptide or the alternative AAV VP1 polypeptide. Such chimeric capsids may be referred to as an “AAV capsid with a GH loop substitution” or a “GH loop substitution capsid.”

In some embodiments, a chimeric AAV capsid polypeptide of the disclosure comprises an amino acid sequence of a parental AAV VP1 polypeptide, comprising a substitution of amino acids from a region between β-sheet G and β-sheet I with amino acids from a region between β-sheet G and β-sheet I of an alternative AAV VP1 polypeptide. In some embodiments, the substitution includes amino acids from the β-sheet G and/or β-sheet I. Such chimeric capsids may be referred to an “AAV capsid with a GI loop substitution” or a “GI loop substitution capsid.”

In some embodiments, novel chimeric AAV capsid polypeptides disclosed herein (e.g., AAV capsids with a GH loop substitution or a GI loop substitution) exhibit advantageous properties, including, for example, a tropism profile differing from the tropism profile of the parental AAV capsid. In some embodiments, novel chimeric AAV capsid polypeptides disclosed herein are amenable to production under standard cell culture protocols and generate titers consistent with that of parental AAV serotypes. In some embodiments, novel chimeric AAV capsid polypeptides disclosed herein are amenable to purification under standard protocols used to generate drug substance for clinical gene therapy program. In some embodiments, novel chimeric capsid polypeptides disclosed herein may be used in the production of rAAV vectors for gene therapy.

Without wishing to be bound by any specific theory, the modified tropism profile of a chimeric capsid may be due to diversity of the 3-fold axis components and the maintenance of production values may be due to binding of the 5-fold axis components with Rep proteins.

In some embodiments, a chimeric AAV capsid polypeptide of the disclosure comprises an amino acid sequence of a parental AAV VP1 polypeptide, comprising a substitution of amino acids from a region between β-sheet G and β-sheet H with amino acids from a region between β-sheet G and β-sheet H of an alternative AAV VP1 polypeptide. In some embodiments, the substitution includes amino acids from the β-sheet G and/or β-sheet H of either the parental AAV VP1 polypeptide or the alternative AAV VP1 polypeptide. In some embodiments, a parental AAV VP1 polypeptide and/or an alternative AAV VP1 polypeptide is from an AAV serotype selected from the group consisting of AAV1, AAV2, AAV3 (including AAV3A and AAV3B), AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2,8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90.

In some embodiments, a chimeric AAV capsid polypeptide of the disclosure comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally, wherein the β-sheet G comprises the amino acid sequence of FX₁X₂X₃YX₄, wherein X₁ is E, Q, or T; wherein X₂ is F, I, or M; wherein X₃ is S, T or V; and wherein X₄ is E, K, N, Q, S or T (SEQ ID NO: 52) and optionally, wherein the β-sheet H comprises the amino acid sequence of X₁X₂X₃IKNT, wherein X₁ is Q, or M; wherein X₂ is I or M; and wherein X₃ is L, M or F (SEQ ID NO: 65). In some embodiments, the substitution includes amino acids from the β-sheet G and/or β-sheet H of either the parental AAV VP1 polypeptide and/or the alternative AAV VP1 polypeptide. In some embodiments, the parental AAV VP1 polypeptide, and/or the alternative AAV VP1 polypeptide comprises an amino acid sequence selected from the group consisting of AAV2 (e.g., SEQ ID NO:1), AAV3B (e.g., SEQ ID NO:3), AAV4 (e.g., SEQ ID NO:5), AAV5 (e.g., SEQ ID NO:7), AAV6 (e.g., SEQ ID NO:9), AAV7 (e.g., SEQ ID NO:11), AAV8 (e.g., SEQ ID NO: 13), AAV9 (e.g., SEQ ID NO:15), AAVrh10 (e.g., SEQ ID NO:12), AAVbovine (e.g., SEQ ID NO: 19), AAVporcine4 (e.g., SEQ ID NO:21) and AAVporcine5 (e.g., SEQ ID NO:13). In some embodiments, the parental AAV VP1 polypeptide, and/or the alternative AAV VP1 polypeptide is encoded by a nucleic acid selected from the group consisting of AAV2 (e.g., SEQ ID NO: 2), AAV3B (e.g., SEQ ID NO:4), AAV4 (e.g., SEQ ID NO:6), AAV5 (e.g., SEQ ID NO:8), AAV6 (e.g., SEQ ID NO:10), AAV7 (e.g., SEQ ID NO:12), AAV8 (e.g., SEQ ID NO:14), AAV9 (e.g., SEQ ID NO:16), AAVrh10 (e.g., SEQ ID NO:18), AAVbovine (e.g., SEQ ID NO:20), AAVporcine4 (e.g., SEQ ID NO:22) and AAVporcine5 (e.g., SEQ ID NO:24).

In some embodiments, a chimeric AAV capsid polypeptide of the disclosure comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally, wherein the β-sheet G comprises the amino acid sequence of FX₁X₂X₃YX₄, wherein X₁ is E, Q, or T; wherein X₂ is F or I; wherein X₃ is S or T; and X₄ is E, N, Q, S or T (SEQ ID NO:53) and optionally, wherein the β-sheet H comprises the amino acid sequence of QX₁X₂IKNT, wherein X₁ is Io M; and wherein X₂ is F, L or M (SEQ ID NO:66). In some embodiments, the substitution includes amino acids from the β-sheet G and/or β-sheet H of either the parental AAV VP1 polypeptide and/or the alternative AAV VP1 polypeptide. In some embodiments, the parental AAV VP1 polypeptide, and/or the alternative AAV VP1 polypeptide comprises an amino acid sequence selected from the group consisting of AAV2 (e.g., SEQ ID NO:1), AAV3B (e.g., SEQ ID NO:3), AAV4 (e.g., SEQ ID NO:5), AAV5 (e.g., SEQ ID NO:7), AAV6 (e.g., SEQ ID NO: 9), AAV7 (e.g., SEQ ID NO:11), AAV8 (e.g., SEQ ID NO:13), AAV9 (e.g., SEQ ID NO: 15), AAVrh10 (e.g., SEQ ID NO:12) and AAVporcine4 (e.g., SEQ ID NO:21). In some embodiments, the parental AAV VP1 polypeptide, and/or the alternative AAV VP1 polypeptide is encoded by a nucleic acid selected from the group consisting of AAV2 (e.g., SEQ ID NO:2), AAV3B (e.g., SEQ ID NO:4), AAV4 (e.g., SEQ ID NO:6), AAV5 (e.g., SEQ ID NO:8), AAV6 (e.g., SEQ ID NO:10), AAV7 (e.g., SEQ ID NO:12), AAV8 (e.g., SEQ ID NO:14), AAV9 (e.g., SEQ ID NO:16), AAVrh10 (e.g., SEQ ID NO:18) and AAVporcine4 (e.g., SEQ ID NO:22).

In some embodiments, a chimeric AAV capsid polypeptide of the disclosure comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally, wherein the region between β-sheet G and β-sheet H of the parental AAV VP1 polypeptide and the region between β-sheet G and β-sheet H of the alternative AAV VP1 polypeptide is flanked by an amino acid sequence comprising or consisting of the sequence VPFHS, (SEQ ID NO:75) at the amino terminal end and is flanked by an amino acid sequence comprising or consisting of the sequence KHPPP (SEQ ID NO:76) or MKHPPP (SEQ ID NO:77) at the carboxy terminal end. In some embodiments, a parental AAV VP1 polypeptide is a AAV9 VP1 polypeptide (e.g., SEQ ID NO:15) or an rh10 VP1 polypeptide (e.g., SEQ IC NO: 17). In some embodiments, an alternative AAV VP1 polypeptide is an AAV2 (e.g., SEQ ID NO:1), AAV3B (e.g., SEQ ID NO:3), AAV4 (e.g., SEQ ID NO:5), AAV5 (e.g., SEQ ID NO:7) or AAV9 VP1 (e.g., SEQ ID NO:15) polypeptide.

In some embodiments, a chimeric AAV capsid polypeptide comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide wherein the chimeric AAV capsid polypeptide comprises an amino acid sequence at least 90%, 95%, 98% or 99% identical to an amino acid sequence of any one of SEQ ID NO:25 (AAV9GH2), SEQ ID NO:27 (AAV9GH3B), SEQ ID NO: 29 (AAV9GH4), SEQ ID NO:31 (AAV9GH5), SEQ ID NO:39 (AAVrh10GH39), and SEQ ID NO: 41 (AAVrh10GH9). In some embodiments, a chimeric AAV capsid polypeptide comprises or consists of an amino acid sequence of any one of SEQ ID NO:25 (AAV9GH2), SEQ ID NO:27 (AAV9GH3B), SEQ ID NO:29 (AAV9GH4), SEQ ID NO:31 (AAV9GH5), SEQ ID NO:39 (AAVrh10GH39), and SEQ ID NO:41 (AAVrh10GH9).

In some embodiments, a chimeric AAV capsid polypeptide comprises an amino acid sequence of a parental AAV VP1 polypeptide, comprising a substitution of amino acids from a region between β-sheet G and β-sheet H with amino acids from a region between β-sheet G, and β-sheet H of an alternative VP1 polypeptide wherein the chimeric AAV capsid polypeptide is encoded by a nucleic acid sequence at least 90%, 95%, 98% or 99% identical to a nucleic acid sequence of any one of SEQ ID NO:26 (AAV9GH2), SEQ ID NO:28 (AAV9GH3B), SEQ ID NO: 30 (AAV9GH4), SEQ ID NO:32 (AAV9GH5), SEQ ID NO:40 (AAVrh10GH4), and SEQ ID NO: 42 (AAVrh10GH9). In some embodiments, a chimeric AAV capsid polypeptide is encoded by a nucleic acid sequence comprising or consisting of a nucleic acid sequence of any one of SEQ ID NO:26 (AAV9GH2), SEQ ID NO:28 (AAV9GH3B), SEQ ID NO:30 (AAV9GH4), SEQ ID NO: 32 (AAV9GH5), SEQ ID NO:40 (AAVrh10GH4), and SEQ ID NO:42 (AAVrh10GH9).

In some embodiments, a chimeric AAV capsid polypeptide of the disclosure comprises an amino acid sequence of an AAV9 VP1 polypeptide, comprising a substitution of amino acids from a region between β-sheet G and β-sheet H with amino acids from a region between β-sheet G and β-sheet H of an AAV2 VP1 polypeptide, wherein the amino acid sequence of the chimeric AAV capsid polypeptide comprises or consists of SEQ ID NO:25.

In some embodiments, a chimeric AAV capsid polypeptide of the disclosure comprises an amino acid sequence of an AAV9 VP1 polypeptide, comprising a substitution of amino acids from a region between β-sheet G and β-sheet H with amino acids from a region between β-sheet G and β-sheet H of an AAV3B VP1 polypeptide, wherein the amino acid sequence of the chimeric AAV capsid polypeptide comprises or consists of SEQ ID NO:27.

In some embodiments, a chimeric AAV capsid polypeptide of the disclosure comprises an amino acid sequence of an AAV9 VP1 polypeptide, comprising a substitution of amino acids from a region between β-sheet G and β-sheet H with amino acids from a region between β-sheet G and β-sheet H of an AAV4 VP1 polypeptide, wherein the amino acid sequence of the chimeric AAV capsid polypeptide comprises or consists of SEQ ID NO:29.

In some embodiments, a chimeric AAV capsid polypeptide of the disclosure comprises an amino acid sequence of an AAV9 VP1 polypeptide, comprising a substitution of amino acids from a region between β-sheet G and β-sheet H with amino acids from a region between β-sheet G and β-sheet H of an AAV5 VP1 polypeptide, wherein the amino acid sequence of the chimeric AAV capsid polypeptide comprises or consists of SEQ ID NO:31.

In some embodiments, a chimeric AAV capsid polypeptide of the disclosure comprises an amino acid sequence of an AAVrh10 VP1 polypeptide, comprising a substitution of amino acids from a region between β-sheet G and β-sheet H with amino acids from a region between β-sheet G and β-sheet H of an AAV4 VP1 polypeptide, wherein the amino acid sequence of the chimeric AAV capsid polypeptide comprises or consists of SEQ ID NO:39.

In some embodiments, a chimeric AAV capsid polypeptide of the disclosure comprises an amino acid sequence of an AAVrh10 VP1 polypeptide, comprising a substitution of amino acids from a region between β-sheet G and β-sheet H with amino acids from a region between β-sheet G and β-sheet H of an AAV9 VP1 polypeptide, wherein the amino acid sequence of the chimeric AAV capsid polypeptide comprises or consists of SEQ ID NO:41.

In some embodiments, a chimeric AAV capsid polypeptide of the disclosure comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally wherein the region between β-sheet G and β-sheet H of the parental AAV VP1 polypeptide and the region between β-sheet G and β-sheet H of the alternative AAV VP1 polypeptide is flanked by an amino acid sequence comprising or consisting of the sequence GNNFX₁F wherein X₁ is E, Q or T (SEQ ID NO:78) at the amino terminal end and flanked by an amino acid sequence comprising or consisting of the sequence KHPPP (SEQ ID NO:76) at the carboxy terminal end.

In some embodiments, the substitution includes amino acids from the β-sheet G and/or β-sheet H of either the parental AAV VP1 polypeptide and/or the alternative AAV VP1 polypeptide. In some embodiments, a parental AAV VP1 polypeptide is a AAV9 VP1 polypeptide (e.g., SEQ ID NO: 15). In some embodiments, an alternative AAV VP1 polypeptide is an AAV6 (e.g., SEQ ID NO: 9), AAV7 (e.g., SEQ ID NO:11) or AAV8 (e.g., SEQ ID NO:13) polypeptide.

In some embodiments, a chimeric AAV capsid polypeptide comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally wherein the substitution includes amino acids from the β-sheet G and/or the β-sheet H, and wherein the chimeric AAV capsid polypeptide comprises an amino acid sequence at least 90%, 95%, 98% or 99% identical to an amino acid sequence of any one of SEQ ID NO:33 (AAV9GH6), SEQ ID NO:35 (AAV9GH7) and SEQ ID NO:37 (AAV9GH8). In some embodiments, a chimeric AAV capsid polypeptide comprises or consists of an amino acid sequence of any one of SEQ ID NO:33 (AAV9GH6), SEQ ID NO:35 (AAV9GH7) and SEQ ID NO:37 (AAV9GH8).

In some embodiments, a chimeric AAV capsid polypeptide comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally wherein the substitution includes amino acids from a β-sheet G and/or the β-sheet H, and wherein the chimeric AAV capsid polypeptide is encoded by a nucleic acid sequence at least 90%, 95%, 98% or 99% identical to a nucleic acid sequence of any one of SEQ ID NO:34 (AAV9GH6), SEQ ID NO:36 (AAV9GH7), SEQ ID NO:38 (AAV9GH8). In some embodiments, a chimeric AAV capsid polypeptide is encoded by a nucleic acid sequence comprising or consisting of a nucleic acid sequence of any one of SEQ ID NO:34 (AAV9GH6), SEQ ID NO:36 (AAV9GH7), SEQ ID NO:38 (AAV9GH8).

In some embodiments, a chimeric AAV capsid polypeptide of the disclosure comprises an amino acid sequence of an AAV9 VP1 polypeptide, comprising a substitution of amino acids from a region between β-sheet G and β-sheet H with amino acids from a region between β-sheet G and β-sheet H of an AAV6 VP1 polypeptide, wherein the amino acid sequence of the chimeric AAV capsid polypeptide comprises or consists of SEQ ID NO:33 and optionally, wherein the substitution includes amino acids from a β-sheet G and/or β-sheet H.

In some embodiments, a chimeric AAV capsid polypeptide of the disclosure comprises an amino acid sequence of an AAV9 VP1 polypeptide, comprising a substitution of amino acids from a region between β-sheet G and β-sheet H with amino acids from a region between β-sheet G and β-sheet H of an AAV7 VP1 polypeptide, wherein the amino acid sequence of the chimeric AAV capsid polypeptide comprises or consists of SEQ ID NO:35 and optionally, wherein the substitution includes amino acids from a β-sheet G and/or β-sheet H.

In some embodiments, a chimeric AAV capsid polypeptide of the disclosure comprises an amino acid sequence of an AAV9 VP1 polypeptide, comprising a substitution of amino acids from a region between β-sheet G and β-sheet H with amino acids from a region between β-sheet G and β-sheet H of an AAV8 VP1 polypeptide, wherein the amino acid sequence of the chimeric AAV capsid polypeptide comprises or consists of SEQ ID NO:37 and optionally, wherein the substitution includes amino acids from a β-sheet G and/or β-sheet H.

In some embodiments, a chimeric AAV capsid polypeptide of the disclosure comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally wherein the region between β-sheet G and β-sheet H of the parental AAV VP1 polypeptide and the region between β-sheet G and β-sheet H of the alternative AAV VP1 polypeptide is flanked by an amino acid sequence comprising or consisting of the sequence LYRFVST (SEQ ID NO: 79) at the amino terminal end and flanked by an amino acid sequence comprising or consisting of the sequence PPPM (SEQ ID NO:80) at the carboxy terminal end. In some embodiments, a parental AAV VP1 polypeptide is a AAV5 VP1 polypeptide (e.g., SEQ ID NO: 7). In some embodiments, an alternative AAV VP1 polypeptide is an AAVporcine5 (e.g., SEQ ID NO:23) polypeptide.

In some embodiments, a chimeric AAV capsid polypeptide comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally wherein the substitution includes amino acids from the β-sheet G and/or the β-sheet H and wherein the chimeric AAV capsid polypeptide comprises an amino acid sequence at least 90%, 95%, 98% or 99% identical to an amino acid sequence of SEQ ID NO:47 (AAV5GHporcine5). In some embodiments, a chimeric AAV capsid polypeptide comprises or consists of an amino acid sequence of SEQ ID NO:47 (AAV5GHporcine5).

In some embodiments, a chimeric AAV capsid polypeptide comprises an amino acid sequence of a parental AAV VP1 polypeptide, comprising a substitution of amino acids from a region between β-sheet G and β-sheet H with amino acids from a region between β-sheet G and β-sheet H of an alternative VP1 polypeptide, optionally wherein the substitution includes amino acids from the β-sheet G and/or the β-sheet H and wherein the chimeric AAV capsid polypeptide is encoded by a nucleic acid sequence at least 90%, 95%, 98% or 99% identical to a nucleic acid sequence of SEQ ID NO:48 (AAV5GHporcine5). In some embodiments, a chimeric AAV capsid polypeptide is encoded by a nucleic acid sequence comprising or consisting of a nucleic acid sequence of SEQ ID NO:48 (AAV5GHporcine5).

In some embodiments, a chimeric AAV capsid polypeptide of the disclosure comprises an amino acid sequence of an AAV5 VP1 polypeptide, comprising a substitution of amino acids from a region between β-sheet G and β-sheet H with amino acids from a region between β-sheet G and β-sheet H of an AAVporcine5 VP1 polypeptide, wherein the amino acid sequence of the chimeric AAV capsid polypeptide comprises or consists of SEQ ID NO:47.

In some embodiments, a chimeric AAV capsid polypeptide of the disclosure comprises an amino acid sequence of a parental AAV VP1 polypeptide, comprising a substitution of amino acids from a region between β-sheet G and β-sheet I with amino acids from a region between β-sheet G and β-sheet I of an alternative AAV VP1 polypeptide. In some embodiments, the substitution includes amino acids from the β-sheet G and/or β-sheet I of either the parental AAV VP1 polypeptide or the alternative AAV VP1 polypeptide. In some embodiments, a parental AAV VP1 polypeptide or an alternative AAV VP1 polypeptide is from an AAV serotype selected from the group consisting of AAV1, AAV2, AAV3 (including AAV3A and AAV3B), AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2,8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90.

In some embodiments, a chimeric AAV capsid polypeptide of the disclosure comprises an amino acid sequence of a parental AAV VP1 polypeptide, comprising a substitution of amino acids from a region between β-sheet G and β-sheet I with amino acids from a region between β-sheet G and β-sheet I of an alternative VP1 polypeptide, optionally, wherein the β-sheet G of the parental AAV VP1 polypeptide and/or of the alternative AAV VP1 polypeptide comprises an amino acid sequence of FX₁X₂X₃YX₄, wherein X₁ is E, Q, or T; wherein X₂ is F, I, or M; wherein X₃ is S, T or V; and X₄ is E, K, N, Q, S or T (SEQ ID NO:52) and optionally, wherein the β-sheet I comprises the amino acid sequence of TQYSTGQVX₁VX₂X₃X₄WEX₅X₆, wherein X₁ is A, S or T; wherein X₂ is E, K or Q; wherein X₃ is I or M; wherein X₄ is D or E; wherein X₅ is I or L; and wherein X₆ is Q or K (SEQ ID NO:71) In some embodiments, the substitution includes amino acids from the β-sheet G and/or β-sheet H of either the parental AAV VP1 polypeptide or the alternative AAV VP1 polypeptide.

In some embodiments, the parental AAV VP1 polypeptide, and/or the alternative AAV VP1 polypeptide comprises an amino acid sequence selected from the group consisting of AAV2 (e.g., SEQ ID NO:1), AAV3B (e.g., SEQ ID NO:3), AAV4 (e.g., SEQ ID NO:5), AAV5 (e.g., SEQ ID NO:7), AAV6 (e.g., SEQ ID NO:9), AAV7 (e.g., SEQ ID NO:11), AAV8 (e.g., SEQ ID NO: 13), AAV9 (e.g., SEQ ID NO:15), AAVrh10 (e.g., SEQ ID NO:12), AAVbovine (e.g., SEQ ID NO: 19), AAVporcine4 (e.g., SEQ ID NO:21) and AAVporcine5 (e.g., SEQ ID NO:13). In some embodiments, the parental AAV VP1 polypeptide, and/or the alternative AAV VP1 polypeptide is encoded by a nucleic acid selected from the group consisting of AAV2 (e.g., SEQ ID NO: 2), AAV3B (e.g., SEQ ID NO:4), AAV4 (e.g., SEQ ID NO:6), AAV5 (e.g., SEQ ID NO:8), AAV6 (e.g., SEQ ID NO:10), AAV7 (e.g., SEQ ID NO:12), AAV8 (e.g., SEQ ID NO:14), AAV9 (e.g., SEQ ID NO:16), AAVrh10 (e.g., SEQ ID NO:18), AAVbovine (e.g., SEQ ID NO:20), AAVporcine4 (e.g., SEQ ID NO:22) and AAVporcine5 (e.g., SEQ ID NO:24).

In some embodiments, a chimeric AAV capsid polypeptide of the disclosure comprises an amino acid sequence of a parental AAV VP1 polypeptide, comprising a substitution of amino acids from a region between β-sheet G and β-sheet I with amino acids from a region between β-sheet G and β-sheet I of an alternative VP1 polypeptide, optionally, wherein the β-sheet G comprises the amino acid sequence of FX₁X₂X₃YX₄, wherein X₁ is E or T; wherein X₂ is F or M; wherein X₃ is S, T or V; and wherein X₄ is K, N or T (SEQ ID NO:81) and optionally, wherein the β-sheet I comprises the amino acid sequence of TQYSTGQVX₁VX₂X₃EWEX₄X₅, wherein X₁ is A, S or T; wherein X₂ is E or K; wherein X₃ is I or M; wherein X₄ is I or L; and wherein X₅ is Q or K (SEQ ID NO:72). In some embodiments, the substitution includes amino acids from the β-sheet G and/or β-sheet H of either the parental AAV VP1 polypeptide or the alternative AAV VP1 polypeptide.

In some embodiments, the parental AAV VP1 polypeptide, and/or the alternative AAV VP1 polypeptide comprises an amino acid sequence selected from the group consisting of AAV5 (e.g., SEQ ID NO:7), AAVbovine (e.g., SEQ ID NO:19), AAVporcine4 (e.g., SEQ ID NO: 21). In some embodiments, the parental AAV VP1 polypeptide, and/or the alternative AAV VP1 polypeptide is encoded by a nucleic acid selected from the group consisting of AAV5 (e.g., SEQ ID NO:8), AAVbovine (e.g., SEQ ID NO:20) and AAVporcine4 (e.g., SEQ ID NO:22).

In some embodiments, a chimeric AAV capsid polypeptide of the disclosure comprises an amino acid sequence of a parental AAV VP1 polypeptide, comprising a substitution of amino acids from a region between β-sheet G and β-sheet I with amino acids from a region between β-sheet G and β-sheet I of an alternative VP1 polypeptide, optionally wherein the region between β-sheet G and β-sheet H of the parental AAV VP1 polypeptide and the region between β-sheet G and β-sheet H of the alternative AAV VP1 polypeptide is flanked by an amino acid sequence comprising or consisting of the sequence MLRTGNNF (SEQ ID NO: 82) at the amino terminal end and is flanked by an amino acid sequence comprising or consisting of the sequence FITQYSTGQV (SEQ ID NO:83) at the carboxy terminal end. In some embodiments, a parental AAV VP1 polypeptide is a AAV5 VP1 polypeptide (e.g., SEQ ID NO: 7). In some embodiments, an alternative AAV VP1 polypeptide is an AAVbovine (e.g., SEQ ID NO: 19) or AAVporcine4 (e.g., SEQ ID NO:21) polypeptide.

In some embodiments, a chimeric AAV capsid polypeptide comprises an amino acid sequence of a parental AAV VP1 polypeptide, comprising a substitution of amino acids from a region between β-sheet G and β-sheet I with amino acids from a region between β-sheet G and β-sheet I of an alternative VP1 polypeptide, optionally wherein the substitution includes amino acids from the β-sheet G and/or the β-sheet H and wherein the chimeric AAV capsid polypeptide comprises an amino acid sequence at least 90%, 95%, 98% or 99% identical to an amino acid sequence of SEQ ID NO:43 (AAV5GHbovine) or SEQ ID NO:45 (AAV5GHporcine4). In some embodiments, a chimeric AAV capsid polypeptide comprises or consists of an amino acid sequence of SEQ ID NO:43 (AAV5GHbovine) or SEQ ID NO:45 (AAV5GHporcine4).

In some embodiments, a chimeric AAV capsid polypeptide comprises an amino acid sequence of a parental AAV VP1 polypeptide, comprising a substitution of amino acids from a region between β-sheet G and β-sheet I with amino acids from a region between β-sheet G and β-sheet I of an alternative VP1 polypeptide, optionally wherein the substitution includes amino acids from the β-sheet G and/or the β-sheet H and wherein the chimeric AAV capsid polypeptide is encoded by a nucleic acid sequence at least 90%, 95%, 98% or 99% identical to a nucleic acid sequence of any one of SEQ ID NO:44 (AAV5GHbovine) or SEQ ID NO:46 (AAV5GHporcine4). In some embodiments, a chimeric AAV capsid polypeptide is encoded by a nucleic acid sequence comprising or consisting of a nucleic acid sequence of any one of SEQ ID SEQ ID NO:44 (AAV5GHbovine) or SEQ ID NO:46 (AAV5GHporcine4).

In some embodiments, a chimeric AAV capsid polypeptide of the disclosure comprises an amino acid sequence of an AAV5 VP1 polypeptide, comprising a substitution of amino acids from a region between β-sheet G and β-sheet I with amino acids from a region between β-sheet G and β-sheet I of an AAVbovine VP1 polypeptide, wherein the amino acid sequence of the chimeric AAV capsid polypeptide comprises or consists of SEQ ID NO:43.

In some embodiments, a chimeric AAV capsid polypeptide of the disclosure comprises an amino acid sequence of an AAV5 VP1 polypeptide, comprising a substitution of amino acids from a region between β-sheet G and β-sheet I with amino acids from a region between β-sheet G and β-sheet I of an AAVporcine4 VP1 polypeptide, wherein the amino acid sequence of the chimeric AAV capsid polypeptide comprises or consists of SEQ ID NO:45.

Chimeric AAV capsids of the disclosure may be produced by methods known to skilled artisans (see, e.g., WO 2013/063379). An exemplary non-limiting method is described in Grieger, et al. (2015) Molecular Therapy 24 (2): 287-297, the contents of which are incorporated by reference herein for all purposes. Chimeric AAV capsids can be made by mammalian cell AAV production systems (e.g., those based on 293T or HEK293 cells) and insect cell AAV production systems (e.g., those based on sf9 insect cells and/or those using baculoviral helper vectors).

Transfection of HEK293 cells allows for rapid and scalable AAV and rAAV production. Using a triple transfection method (e.g., WO 96/40240), with a packaging cell, such as HEK293, a plasmid encoding an AAV rep and chimeric capsid of the disclosure, a plasmid encoding helper functions (e.g., adenovirus or HSV proteins such as Ela, E1b, E2a, E4, and VA RNA), and a plasmid encoding a transgene (e.g., a therapeutic transgene, a reporter transgene (e.g., green fluorescent protein) and various elements to control expression of the transgene, rAAV vectors comprising a chimeric capsid of the disclosure can be produced.

In some embodiments, a host cell transfected with a nucleic acid encoding a chimeric AAV capsid polypeptide comprising an amino acid sequence of a parental AAV VP1 polypeptide, comprising a substitution of amino acids from a region between β-sheet G and β-sheet H (or β-sheet I) with amino acids from a region between β-sheet G and β-sheet H (or β-sheet I) of an alternative VP1 polypeptide and a plasmid comprising a transgene produces between about 1E+1 vg/cell to about 1E+10 vg/cell. In some embodiments, a host cell transfected with a plasmid comprising a nucleic acid (e.g., SEQ ID NO:26, 28, 30, 32, 34, 36, 38, 40, 42, 44, 46 or 48) encoding a chimeric AAV capsid polypeptide and a plasmid comprising a transgene produces between about 1E+1 vg/cell, about 1E+2 vg/cell, about 1E+3 vg/cell, about 1E+4 vg/cell, 1E+5 vg/cell, about 1E+6 vg/cell, about 1E+7 vg/cell, about 1E+8, vg/cell, about 1E+9 vg/cell or about 1E+10 vg/cell.

In some embodiments, a host cell transfected with plasmid comprising a nucleic acid encoding a chimeric AAV capsid polypeptide (e.g., SEQ ID NO:26, 28, 30, 32, 34, 36, 38, 40, 42, 44, 46 or 48) and a plasmid comprising a transgene produces an amount of rAAV vector that is substantially similar to an amount of rAAV vector made by an otherwise identical host cell comprising a plasmid comprising a nucleic acid encoding an AAV capsid polypeptide of a parental serotype (e.g., AAV9).

In some embodiments, a host cell transfected with a plasmid comprising a nucleic acid (e.g., SEQ ID NO:26, 28, 30, 32, 34, 36, 38, 40, 42, 44, 46 or 48) encoding a chimeric AAV capsid polypeptide and a plasmid comprising a transgene produces an amount of rAAV vector that about 80% to 140% of an amount of rAAV vector made by an otherwise identical host cell comprising a plasmid comprising a nucleic acid encoding an AAV capsid polypeptide of a parental serotype (e.g., AAV9).

In some embodiments, a host cell transfected with a plasmid comprising a nucleic acid (e.g., SEQ ID NO: 34, 36, 38,) encoding a chimeric AAV capsid polypeptide and a plasmid comprising a transgene produces an amount of rAAV vector about 80% to 140% of an amount of rAAV vector made by an otherwise identical host cell comprising a plasmid comprising a nucleic acid encoding an AAV9 capsid polypeptide.

In some embodiments, a host cell transfected with a plasmid comprising a nucleic acid encoding a chimeric AAV capsid comprising a parental AAV9 amino acid sequence and an AAV6 GH loop substitution (e.g., SEQ ID NO:33) produces a substantially similar amount of vg/cell as compared to an amount of vg/cell produced by a host cell transfected with a plasmid comprising a nucleic acid encoding an AAV9 capsid (e.g., SEQ ID NO:15). In some embodiments, a host cell transfected with a plasmid comprising a nucleic acid encoding a chimeric AAV capsid comprising a parental AAV9 amino acid sequence and an AAV6 GH loop substitution (e.g., SEQ ID NO:33) produces more than 1E+3 vg/cell.

In some embodiments, a host cell transfected with a plasmid comprising a nucleic acid encoding a chimeric AAV capsid comprising a parental AAV9 amino acid sequence and an AAV7 GH loop substitution (e.g., SEQ ID NO:35) produces a substantially similar amount of vg/cell as compared to an amount of vg/cell produced by a host cell transfected with a plasmid comprising a nucleic acid encoding an AAV9 capsid (e.g., SEQ ID NO:15). In some embodiments, a host cell transfected with a plasmid comprising a nucleic acid encoding a chimeric AAV capsid comprising a parental AAV9 amino acid sequence and an AAV7 GH loop substitution (e.g., SEQ ID NO:35) produces more than 1E+3 vg/cell. In some embodiments, a host cell transfected with a plasmid comprising a nucleic acid encoding a chimeric AAV capsid comprising a parental AAV9 amino acid sequence and an AAV7 GH loop substitution (e.g., SEQ ID NO:35) produces more than 1E+10 vg/mL.

In some embodiments, a host cell transfected with a plasmid comprising a nucleic acid encoding a chimeric AAV capsid comprising a parental AAV9 amino acid sequence and an AAV8 GH loop substitution (e.g., SEQ ID NO:37) produces a substantially similar amount of vg/cell as compared to an amount of vg/cell produced by a host cell transfected with a plasmid comprising a nucleic acid encoding an AAV9 capsid (e.g., SEQ ID NO:15). In some embodiments, a host cell transfected with a plasmid comprising a nucleic acid encoding a chimeric AAV capsid comprising a parental AAV9 amino acid sequence and an AAV8 GH loop substitution (e.g., SEQ ID NO:37) produces more than 1E+3 vg/cell. In some embodiments, a host cell transfected with a plasmid comprising a nucleic acid encoding a chimeric AAV capsid comprising a parental AAV9 amino acid sequence and an AAV8 GH loop substitution (e.g., SEQ ID NO:37) produces more than 1E+10 vg/mL.

In some embodiments, a host cell transfected with a plasmid comprising a nucleic acid encoding a chimeric AAV capsid comprising a parental AAVrh10 amino acid sequence and an AAV9 GH loop substitution (e.g., SEQ ID NO:41), produces more than 1.5E+1 vg/cell.

In some embodiments, a host cell transfected with plasmid comprising a nucleic acid encoding a chimeric AAV capsid polypeptide (e.g., SEQ ID NO:26, 28, 30, 32, 34, 36, 38, 40, 42, 44, 46 or 48) and a plasmid comprising a transgene produces an amount of rAAV vector that is 5 to 100-fold greater than an amount of rAAV vector made by an otherwise identical host cell comprising a plasmid comprising a nucleic acid encoding an AAV capsid polypeptide of a parental serotype.

In some embodiments, chimeric vectors have been engineered to exhibit altered tropism or tropism for a particular tissue or cell type. The term “tropism” refers to preferential entry of the virus into certain cell or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Tissue tropism and biodistribution of AAV serotypes have been studied in vivo and have shown preferential tropism of AAV8 for liver, AAV1, AAV6 and AAV9 for heart, AAV1, AAV6 and AAV9 for skeletal muscle, AAV5 for lung, AAV1, AAV5, AAV8 and AAV9 for the CNS and AAV4 and AAV8 for the eye (Asokan et al. (2012) Mol. Therapy 20 (4): 699-708).

In some embodiments, tissue tropism of an AVV capsid of the disclosure, i.e., an AAV capsid with a GH loop substitution or a GI loop substitution can be demonstrated by administration of a rAAV vector comprising the chimeric capsid and a reporter transgene (e.g., GFP) to an animal, for example, a mouse.

A “tropism profile” refers to a pattern of transduction of one or more target cells, tissues and/or organs. For example, an AAV capsid may have a tropism profile characterized by efficient transduction of muscle cells with only low transduction of, for example, brain cells.

In some embodiments, a chimeric capsid of the disclosure comprising a GH loop substitution, has a tropism profile that differs from the parent AAV capsid. In some embodiments, an AAV9 capsid comprising an AAV6 GH loop substitution (e.g., comprising or consisting of an amino acid of SEQ ID NO:33) has tropism for skeletal muscle and or cardiac muscle. In some embodiments, an AAV9 capsid comprising an AAV 6 GH loop substitution (e.g., comprising or consisting of an amino acid of SEQ ID NO:33) has reduced tropism for liver tissue as compared to an AAV9 capsid or an AAV7 capsid. In some embodiments, an AAV9 capsid comprising an AAV 6 GH loop substitution (e.g., comprising or consisting of an amino acid of SEQ ID NO:33) has reduced tropism for brain tissue as compared to the tropism of an AAV9 capsid or an AAV7 capsid.

In some embodiments, an AAV9 capsid comprising an AAV7 GH loop substitution (e.g., comprising or consisting of an amino acid of SEQ ID NO:35) has tropism for skeletal muscle, cardiac muscle and liver.

In some embodiments, an AAV9 capsid comprising an AAV8 GH loop substitution (e.g., comprising or consisting of an amino acid of SEQ ID NO:37) has reduced tropism for liver, heart and muscle as compared to the tropism of an AAV9 capsid.

In some embodiments, an an AAV9 capsid comprising an AAV7 GH loop substitution (e.g., comprising or consisting of an amino acid of SEQ ID NO:35) has tropism for brain, heart, skeletal muscle, liver and spine, optionally when administer by ICV.

C. Recombinant AAV (rAAV)

A “recombinant adeno-associated virus,” or “rAAV” (also referred to herein as a “rAAV vector,” “rAAV viral particle,” and/or “rAAV vector particle”) refers to an AAV capsid comprising a vector genome, unless specifically noted otherwise. The vector genome comprises a polynucleotide sequence that is not, at least in part, derived from a naturally-occurring AAV (e.g., a heterologous polynucleotide not present in wild type AAV), and wherein the rep and/or cap genes of the wild type AAV genome have been removed from the vector genome. ITRs from an AAV have been added or remain in the vector genome. Therefore, the term rAAV vector encompasses a rAAV viral particle that comprises a capsid (including a chimeric capsid as disclosed herein) but does not comprise a complete AAV genome; instead the recombinant viral particle can comprise a heterologous, i.e., not originally present in the capsid, nucleic acid, the vector genome. Thus, a “rAAV vector genome” (or “vector genome”) refers to a heterologous polynucleotide sequence (including at least one ITR) that may, but need not, be contained within an AAV capsid. A rAAV vector genome may be double-stranded (dsAAV), single-stranded (ssAAV) or self-complementary (scAAV). Typically, a vector genome comprises a heterologous nucleic acid often encoding a therapeutic transgene, or a reporter transgene such as eGFP.

A rAAV vector, and those terms provided above, are to be distinguished from an “AAV viral particle” or “AAV virus” that is not recombinant, contains a virus genome encoding rep and cap genes, and which AAV virus is capable of replicating when present in a cell also comprising a helper virus, such as an adenovirus and/or herpes simplex virus, and/or required helper genes therefrom. Thus, production of a rAAV vector necessarily includes production of a recombinant vector genome using recombinant DNA technologies, and wherein the recombinant vector genome is contained within an AAV capsid to form the rAAV vector.

The present disclosure provides for chimeric AAV capsids comprising GH loop or GI loop substitutions and methods of use thereof, including as a rAAV vector. In some embodiments, delivery or administration of a rAAV vector to a subject (e.g. a patient) provides encoded proteins and peptides to the subject. Thus, a rAAV vector comprising a chimeric capsid comprising or consisting of an amino acid of any one of SEQ ID NO:25, SEQ ID NO:27, SEQ ID NO:29, SEQ ID NO:31, SEQ ID NO:33, SEQ ID NO:35, SEQ ID NO:37, SEQ ID NO: 39, SEQ ID NO:41, SEQ ID NO:43, SEQ ID NO:45 and SEQ ID NO:47 can be used to transfer/deliver a heterologous polynucleotide for expression for the treatment of diseases, disorders and/or conditions.

A rAAV vector genome generally retains 130 to 145 base ITRs in cis to the heterologous nucleic acid sequence that replaces the viral rep and cap genes. Such ITRs are necessary to produce a recombinant AAV vector as they mediate AAV genome replication and packaging. However, modified AAV ITRs and non-AAV terminal repeats including partially or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. A rAAV vector genome optionally comprises two ITRs which are generally at the 5′ and 3′ ends of the vector genome comprising a heterologous sequence (e.g., a transgene encoding a gene of interest). A 5′ and a 3′ ITR may both comprise the same sequence, or each may comprise a different sequence.

A rAAV vector genome may comprise an ITR from an AAV serotype (e.g., wild-type AAV2, a fragment or variant thereof) that differs from the serotype of a parental AAV VP1 polypeptide (e.g., AAV9, AAV5, AAVrh10) and/or an alternative AAV VP1 polypeptide (e.g., AAV2, AAV2B, AAV4, AAV5, AAV6, AAV7, AAV8, AAV9, AAVbovine, AAVporcine4, AAVporcine5). Such a rAAV vector genome comprising at least one ITR from one serotype, but comprising a capsid from a different serotype, may be referred to as a hybrid viral vector (see U.S. Pat. No. 7,172,893; Rabinowitz et al. (2002) J. Virology 76 (2): 791-801). An rAAV ITR may include the entire wild type ITR sequence, or be a variant, fragment, or modification thereof, but will retain functionality.

In addition to a transgene and at least one ITR, a vector genome may also include various regulatory or control elements. Typically, regulatory elements are nucleic acid sequence(s) that influence expression of an operably linked polynucleotide (e.g., a transgene). The precise nature of regulatory elements useful for gene expression will vary from organism to organism and from cell type to cell type including, for example, a promoter, enhancer, intron etc., with the intent to facilitate proper heterologous polynucleotide transcription and translation. Regulatory control can be affected at the level of transcription, translation, splicing, message stability, etc. In some embodiments, a rAAV vector comprising a recombinant nucleic acid comprises at least one ITR, a transgene, a promoter and a polyadenylation signal (polyA) sequence.

A chimeric AAV capsid polypeptide of the disclosure may comprise an amino acid sequence of a parental AAV VP1 polypeptide, or an alternative AAV VP1 polypeptide, selected from the group consisting of AAV1, AAV2, AAV3 (including AAV3A and AAV3B, AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2,8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90.

The rAAV vectors described herein may be obtained by any known production systems, such as mammalian cell AAV production systems (e.g., those based on 293T or HEK293 cells) and insect cell AAV production systems (e.g., those based on sf9 insect cells and/or those using baculoviral helper vectors).

A rAAV vector may be purified by methods standard in the art such as by any number of column chromatography methods (e.g., affinity chromatography, ion exchange chromatography, hydrophobic interaction chromatography) or cesium chloride gradients. Methods for purifying rAAV vectors are known in the art and include methods described in Clark et al. (1999) Human Gene Therapy 10 (6): 1031-1039; Schenpp et al. (2002) Methods Mol. Med. 69:427-443; U.S. Pat. No. 6,566,118 and WO 98/09657.

After rAAV vectors have been produced and purified, they can be titered (e.g., the amount of rAAV vector in a sample can be quantified) to prepare compositions for administration to subjects, such as human subjects with a disease. rAAV vector titering can be accomplished using methods know in the art.

Unless otherwise defined herein, scientific and technical terms used in connection with the present disclosure shall have the meanings that are commonly understood by those of ordinary skill in the art. All publications and other references mentioned herein are incorporated by reference in their entirety. Although a number of documents are cited herein, this citation does not constitute an admission that any of these documents form part of the common general knowledge in the art.

### Embodiments

Those skilled in the art will recognize or be able to ascertain using no more than routine experimentation, many equivalents to the specific embodiments of the invention described herein. Such equivalents are intended to be encompassed by the following embodiments (E).

E1. An AAV capsid polypeptide comprising an amino acid sequence of a parental AAV VP1 polypeptide, comprising a substitution of amino acids from a region between β-sheet G and β-sheet H with amino acids from a region between β-sheet G and β-sheet H of an alternative AAV VP1 polypeptide.

E2. The AAV capsid polypeptide of E1, wherein the region between β-sheet G and β-sheet H of the parental AAV VP1polypeptide and the alternative AAV VP1 polypeptide comprises about 222 to about 235 amino acids.

E3. The AAV capsid polypeptide of E1, wherein the regions between β-sheet G and β-sheet H comprise amino acids from within β-sheet G and β-sheet H.

E4. The AAV capsid polypeptide of E1 or E3, wherein the region within and between β-sheet G and β-sheet H of the parental AAV VP1polypeptide and the alternative AAV VP1 polypeptide comprises about 235 to about 248 amino acids.

E5. The AAV capsid polypeptide of any one of E1-E4, wherein the parental AAV VP1 and/or the alternative AAV VP1 is selected from the group consisting of AAV2, AAV3 (including AAV3A and AAV3B), AAV4, AAV5, AAV6, AAV7, AAV8, AAV9, AAVrh10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2,8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90.

E6. The AAV capsid polypeptide any one of E1-E5, wherein β-sheet G of the parental AAV VP1polypeptide and/or β-sheet G of the alternative AAV VP1 polypeptide comprises or consists of an amino acid sequence of FX₁X₂X₃YX₄, wherein X₁ is E, Q, or T; wherein X₂ is F, I, or M; wherein X₃ is S, T or V; and wherein X₄ is E, K, N, Q, S or T (SEQ ID NO:52).

E7. The AAV capsid polypeptide of E6, wherein the parental AAV VP1 and/or the alternative AAV VP1 is selected from the group consisting of AAV2, AAV3B, AAV4, AAV5, AAV6, AAV7, AAV8, AAV9, AAVrh10, AAVbovine, AAVporcine4 and AAVporcine5.

E8. The AAV capsid polypeptide any one of E1-E7, wherein β-sheet G of the parental AAV VP1polypeptide and/or β-sheet G of the alternative AAV VP1 polypeptide comprises or consists of an amino acid sequence of FX₁X₂X₃YX₄, wherein X₁ is E, Q, or T; wherein X₂ is F or; wherein X₃ is S or T; and X₄ is E, Q, S or T (SEQ ID NO:53).

E9. The AAV capsid polypeptide of E8, wherein the parental AAV VP1 and/or the alternative AAV VP1 is selected from the group consisting of AAV2, AAV3B, AAV4, AAV6, AAV7, AAV8, AAV9, AAVrh10, AAVporcine4 and AAVporcine5.

E10. The AAV capsid polypeptide any one of E1-E9, wherein β-sheet H of the parental AAV VP1 polypeptide and/or β-sheet H of the alternative AAV VP1 polypeptide comprises or consists of an amino acid sequence of X₁X₂X₃IKNT, wherein X₁ is Q, or M; wherein X₂ is I or M; and wherein X₃ is L, M or F (SEQ ID NO:65).

E11. The AAV capsid polypeptide of E10, wherein the parental AAV VP1 and/or the alternative AAV VP1 is selected from the group consisting of AAV2, AAV3B, AAV4, AAV5, AAV6, AAV7, AAV8, AAV9, AAVrh10, AAVbovine, AAVporcine4 and AAVporcine5.

E12. The AAV capsid polypeptide of any one of E1-E11, wherein β-sheet H of the parental AAV VP1 polypeptide and/or β-sheet H of the alternative AAV VP1 polypeptide comprises or consists of an amino acid sequence of QX₁X₂IKNT, wherein X₁ is I or M; and wherein X₂ is F, L or M (SEQ ID NO:66).

E13. The AAV capsid polypeptide of any one of E12, wherein a serotype of the parental AAV VP1 polypeptide and/or the alternative AAV VP1 is selected from the group consisting of AAV2, AAV3B, AAV4, AAV6, AAV7, AAV8, AAV9, AAVrh10, AAVbovine and AAVporcine4.

E14. The AAV capsid polypeptide of any one of E1-E13, wherein the amino acid sequence of the parental AAV VP1 polypeptide and/or of the alternative AAV VP1 polypeptide is set forth in any one of SEQ ID NO:1, SEQ ID NO:3, SEQ ID NO:5, SEQ ID NO:7, SEQ ID NO:9, SEQ ID NO: 11, SEQ ID NO: 13, SEQ ID NO:15, SEQ ID NO:17, SEQ ID NO: 19, SEQ ID NO:21 and SEQ ID NO:23.

E15. The AAV capsid polypeptide of any one of E1-E14, wherein the amino acid sequence of the parental AAV VP1 polypeptide and/or of the alternative AAV VP1 polypeptide is encoded by a nucleic acid set forth in any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO: 20, SEQ ID NO:22 and SEQ ID NO:24.

E16. The AAV capsid polypeptide of any one of E1-E15, wherein the region between β-sheet G and β-sheet H of the parental AAV VP1 polypeptide and/or the region between β-sheet G and β-sheet H of the alternative AAV VP1 polypeptide is flanked by an amino acid sequence comprising or consisting of the sequence VPFHS, (SEQ ID NO:75) at the amino terminal end and is flanked by an amino acid sequence comprising or consisting of the sequence KHPPP (SEQ ID NO:76) or MKHPPP (SEQ ID NO:77) at the carboxy terminal end.

E17. The AAV capsid polypeptide of any one of E1-E16, wherein the serotype of the parental AAV VP1 polypeptide is AAV9 or AAVrh10 and wherein the serotype of the alternative AAV

VP1 polypeptide is selected from the group consisting of AAV2, AAV3B, AAV4, AAV5 and AAV9.

E18. The AAV capsid polypeptide of any one of E1-E17, wherein the polypeptide comprises an amino acid sequence at least 90%, 95%, 98% or 99% identical to an amino acid sequence of any one of SEQ ID NO:25, SEQ ID NO:27, SEQ ID NO:29, SEQ ID NO:31, SEQ ID NO:39, and SEQ ID NO:41.

E19. The AAV capsid polypeptide of any one of E1-E18, wherein the polypeptide comprises or consists of the amino acid sequence of any one of SEQ ID NO:25, SEQ ID NO:27, SEQ ID NO: 29, SEQ ID NO:31, SEQ ID NO:39, and SEQ ID NO:41.

E20. The AAV capsid polypeptide of any one of E1-E19, wherein the polypeptide is encoded by a nucleic acid sequence at least 90%, 95%, 98% or 99% identical to a nucleic acid sequence of any one of SEQ ID NO:26, SEQ ID NO:28, SEQ ID NO:30, SEQ ID NO:32, SEQ ID NO: 40, and SEQ ID NO:42.

E21. The AAV capsid polypeptide of any one of E1-E20, wherein the polypeptide is encoded by a nucleic acid sequence comprising or consisting of any one of SEQ ID NO:26, SEQ ID NO: 28, SEQ ID NO:30, SEQ ID NO:32, SEQ ID NO:40, and SEQ ID NO:42.

E22. An AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV2 VP1 polypeptide, and wherein the polypeptide comprises the amino acid sequence of SEQ ID NO: 25.

E23. An AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV3B VP1 polypeptide, and wherein the polypeptide comprises the amino acid sequence of SEQ ID NO: 27.

E24. An AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV4 VP1 polypeptide, and wherein the polypeptide comprises the amino acid sequence of SEQ ID NO: 29.

E25. An AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV5 VP1 polypeptide, and wherein the polypeptide comprises the amino acid sequence of SEQ ID NO: 31.

E26. An AAV capsid polypeptide comprising an amino acid sequence of an AAVrh10 VP1 polypeptide, comprising a substitution of amino acids from a region between β-sheet G and β-sheet H with amino acids from a region between β-sheet G and β-sheet H of an AAV4 VP1 polypeptide, and wherein the polypeptide comprises the amino acid sequence of SEQ ID NO: 39.

E27. An AAV capsid polypeptide comprising an amino acid sequence of an AAVrh10 VP1 polypeptide, comprising a substitution of amino acids from a region between β-sheet G and β-sheet H with amino acids from a region between β-sheet G and β-sheet H of an AAV9 VP1 polypeptide, and wherein the polypeptide comprises the amino acid sequence of SEQ ID NO: 41.

E28. The AAV capsid polypeptide of any one of E1-E15, wherein the region between β-sheet G and β-sheet H of the parental AAV VP1 polypeptide and/or the region between β-sheet G and β-sheet H of the alternative AAV VP1 polypeptide is flanked by an amino acid sequence comprising or consisting of the sequence GNNFX₁F wherein X₁ is E, Q or T (SEQ ID NO:78) at the amino terminal end and flanked by an amino acid sequence comprising or consisting of the sequence KHPPP (SEQ ID NO:76) at the carboxy terminal end.

E29. The AAV capsid polypeptide of any one of E1-E15 or E28, wherein the serotype of the parental AAV VP1 polypeptide is AAV9 and wherein the serotype of the alternative AAV VP1 polypeptide is selected from the group consisting of AAV6, AAV7 and AAV8.

E30. The AAV capsid polypeptide of any one of E1-E15, E28 or E29, wherein the polypeptide comprises an amino acid sequence at least 90%, 95%, 98% or 99% identical to any one of SEQ ID NO:33, SEQ ID NO:35 and SEQ ID NO:37.

E31. The AAV capsid polypeptide of any one of E1-E15 or E28-E30, wherein the polypeptide comprises or consists of the amino acid sequence of any one of SEQ ID NO:33, SEQ ID NO:35 and SEQ ID NO:37.

E32. The AAV capsid polypeptide of any one of E1-E15 or E28-E31, wherein the polypeptide is encoded by a nucleic acid sequence at least 90%, 95%, 98% or 99% identical to a nucleic acid sequence of any one of SEQ ID NO:34, SEQ ID NO:36 and SEQ ID NO:38.

E33. The AAV capsid polypeptide of any one of E1-E15 or E28-E32, wherein the polypeptide is encoded by a nucleic acid sequence comprising or consisting of a nucleic acid sequence of any one of SEQ ID NO:34, SEQ ID NO:36 and SEQ ID NO:38.

E34. An AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV6 VP1 polypeptide, and wherein the polypeptide comprises the amino acid sequence of SEQ ID NO: 33.

E35. An AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV7 VP1 polypeptide, and wherein the polypeptide comprises the amino acid sequence of SEQ ID NO: 35.

E36. An AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV8 VP1 polypeptide, and wherein the polypeptide comprises the amino acid sequence of SEQ ID NO: 37.

E37. The AAV capsid polypeptide of any one of E1-E15, wherein the region between β-sheet G and β-sheet H of the parental AAV VP1polypeptide and/or the region between β-sheet G and β-sheet H of the alternative AAV VP1 polypeptide is flanked by an amino acid sequence comprising or consisting of the sequence LYRFVST (SEQ ID NO:79) at the amino terminal end and flanked by an amino acid sequence comprising or consisting of the sequence PPPM (SEQ ID NO: 80) at the carboxy terminal end.

E38. The AAV capsid polypeptide of any one of E1-E15 or E37, wherein the serotype of the parental AAV VP1 polypeptide is AAV5 and wherein the serotype of the alternative AAV VP1 polypeptide is AAVporcine5.

E39. The AAV capsid polypeptide of any one of E1-E15 or E37-E38, wherein the polypeptide comprises an amino acid sequence at least 90%, 95%, 98% or 99% identical to SEQ ID NO:47.

E40. The AAV capsid polypeptide of any one of E1-E15 or E37-E39, wherein the polypeptide comprises or consists of the amino acid sequence of SEQ ID NO:47.

E41. The AAV capsid polypeptide of any one of E1-E15 or E37-E40, wherein the polypeptide is encoded by a nucleic acid at least 90%, 95%, 98% or 99% identical to SEQ ID NO:48.

E42. The AAV capsid polypeptide of any one of E1-E15 or E37-E41, wherein the polypeptide is encoded by a nucleic acid comprising or consisting of the nucleic acid of SEQ ID NO:48.

E43. An AAV capsid polypeptide comprising an amino acid sequence of an AAV5 VP1 polypeptide, comprising a substitution of amino acids from a region between β-sheet G and β-sheet H with amino acids from a region between β-sheet G and β-sheet H of an AAVporcine5 VP1 polypeptide, and wherein the polypeptide comprises the amino acid sequence of SEQ ID NO: 47.

E44. An AAV capsid polypeptide comprising an amino acid sequence of a parental AAV VP1 polypeptide, comprising a substitution of amino acids from a region between β-sheet G and β-sheet I with amino acids from a region between β-sheet G and β-sheet I of an alternative AAV VP1 polypeptide.

E45. The AAV capsid polypeptide of E44, wherein the parental AAV VP1 and/or the alternative AAV VP1 is selected from the group consisting of AAV1, AAV2, AAV3 (including AAV3A and AAV3B, AAV4, AAV5, AAV6, AAV7, AAV8, AAV9, AAV10, AAV12, AAVrh8, AAVrh10, AAVrh39, AAVrh43, AAVrh74, AAVrh32.22, AAV1.1, AAV2.5, AAV6.1, AAV6.2, AAV6.3.1, AAV9.45, AAVShH10, HSC15/17, RHM4-1, RHM15-1, RHM15-2, RHM15-3/RHM15-5, RHM15-4, RHM15-6, AAVhu.26, AAV2i8, AAV29G, AAV2,8G9, AAV-LK03, AAV2-TT, AAV2-TT-S312N, AAV3B-S312N, AAVavian, AAVbat, AAVbovine, AAVcanine, AAVequine, AAVprimate, AAVnon-primate, AAVovine, AAVmuscovy duck, AAVporcine4, AAVporcine5, AAVsnake NP4, NP22, NP66, AAVDJ, AAVDJ/8, AAVDJ/9, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVv66, AAVv33, AAVv37, AAVv40, AAVv67, AAVv70, AAVv72, AAVv84, AAVv86, AAVv87 and AAVv90.

E46. The AAV capsid polypeptide of E44-E45, wherein the regions between β-sheet G and β-sheet I comprise amino acids from within β-sheet G and β-sheet I.

E47. The AAV capsid polypeptide of E44-E46, wherein the β-sheet G of the parental AAV VP1 polypeptide and/or of the alternative AAV VP1 polypeptide comprises or consists of an amino acid sequence of FX₁X₂X₃YX₄, wherein X₁ is E or T; wherein X₂ is F or M; wherein X₃ is S, T or V; and wherein X₄ is K, N or T (SEQ ID NO:81).

E48. The AAV capsid polypeptide of E47, wherein the parental AAV VP1 and/or the alternative AAV VP1 is selected from the group consisting of AAV2, AAV5, AAV6, AAV8, AAVbovine, AAVporcine4 and AAVporcine5.

E49. The AAV capsid polypeptide of E44-E48, wherein the β-sheet I of the parental AAV VP1 polypeptide and/or of the alternative AAV VP1 polypeptide comprises or consists of an amino acid sequence of TQYSTGQVX₁VX₂X₃X₄WEX₅X₆, wherein X₁ is A, S or T; wherein X₂ is E, K or Q; wherein X₃ is I or M; wherein X₄ is D or E; wherein X₅ is I or L; and wherein X₆ is Q or K (SEQ ID NO:71).

E50. The AAV capsid polypeptide of E49, wherein the parental AAV VP1 and/or the alternative AAV VP1 is selected from the group consisting of AAV2, AAV3B, AAV4, AAV5, AAV6, AAV7, AAV8, AAV9, AAVrh10, AAVbovine, AAVporcine4 and AAVporcine5.

E51. The AAV capsid polypeptide of E44-E50, wherein the β-sheet I of the parental AAV VP1 polypeptide and/or of the alternative AAV VP1 polypeptide comprises or consists of an amino acid sequence of TQYSTGQVX₁VX₂X₃EWEX₄X₅, wherein X₁ is A, S or T; wherein X₂ is E or K; wherein X₃ is I or M; wherein X₄ is I or L; and wherein X₅ is Q or K (SEQ ID NO:72).

E52. The AAV capsid polypeptide of E51, wherein the parental AAV VP1 and/or the alternative AAV VP1 is selected from the group consisting of AAV2, AAV3B, AAV5, AAV6, AAV7, AAV8, AAV9, AAVrh10, AAVbovine, AAVporcine4 and AAVporcine5.

E53. The AAV capsid polypeptide of any one of E44-E52, wherein the amino acid sequence of the parental AAV VP1 polypeptide and/or of the alternative AAV VP1 polypeptide is set forth in any one of SEQ ID NO:1, SEQ ID NO:3, SEQ ID NO:7, SEQ ID NO:9, SEQ ID NO:11, SEQ ID NO: 13, SEQ ID NO: 15, SEQ ID NO: 17, SEQ ID NO: 19, SEQ ID NO:21 and SEQ ID NO:23.

E54. The AAV capsid polypeptide of any one of E44-E53, wherein the amino acid sequence of the parental AAV VP1 polypeptide and/or of the alternative AAV VP1 polypeptide is encoded by a nucleic acid set forth in any one of SEQ ID NO:2, SEQ ID NO:4, SEQ ID NO:8, SEQ ID NO: 10, SEQ ID NO: 12, SEQ ID NO:14, SEQ ID NO:16, SEQ ID NO:18, SEQ ID NO: 20, SEQ ID NO: 22 and SEQ ID NO:24.

E55. The AAV capsid polypeptide of any one of E44-E54, wherein the region between β-sheet G and β-sheet I of the parental AAV VP1polypeptide and the region between β-sheet G and β-sheet I of the alternative AAV VP1 polypeptide is flanked by an amino acid sequence comprising or consisting of the sequence MLRTGNNF (SEQ ID NO:82) at the amino terminal end and flanked by an amino acid sequence comprising or consisting of the sequence FITQYSTGQV (SEQ ID NO:83) at the carboxy terminal end.

E56. The AAV capsid polypeptide of E33, wherein the serotype of the parental AAV VP1 polypeptide is AAV5 and wherein the serotype of the alternative AAV VP1 polypeptide is AAVbovine or AAVporcine4.

E57. The AAV capsid polypeptide of any one of E44-E56, wherein the polypeptide comprises an amino acid sequence at least 90%, 95%, 98% or 99% identical to SEQ ID NO:43 or SEQ ID NO: 45.

E58. The AAV capsid polypeptide of any one of E44-E57, wherein the polypeptide comprises or consists of the amino acid sequence of SEQ ID NO:43 or SEQ ID NO:45.

E59. The AAV capsid polypeptide of any one of E44-E58, wherein the polypeptide is encoded by a nucleic acid at least 90%, 95%, 98% or 99% identical to SEQ ID NO:44 or SEQ ID NO:46.

E60. The AAV capsid polypeptide of any one of E44-E59, wherein the polypeptide is encoded by a nucleic acid comprising or consisting of SEQ ID NO:44 or SEQ ID NO:46.

E61. An AAV capsid polypeptide comprising an amino acid sequence of an AAV5 VP1 polypeptide, comprising a substitution of amino acids from a region between β-sheet G and β-sheet I with amino acids from a region between β-sheet G and β-sheet I of an AAVbovine VP1 polypeptide, and wherein the polypeptide comprises the amino acid sequence of SEQ ID NO: 43.

E62. An AAV capsid polypeptide comprising an amino acid sequence of an AAV5 VP1 polypeptide, comprising a substitution of amino acids from a region between β-sheet G and β-sheet I with amino acids from a region between β-sheet G and β-sheet I of an AAVporcine4 VP1 polypeptide, and wherein the polypeptide comprises the amino acid sequence of SEQ ID NO: 46.

E63. The AAV capsid polypeptide of any one of E28-E34, wherein any one or more of amino acids E547 S552, N553, A555, N558 V559 from the AAV6 VP1 polypeptide and wherein any one or more of amino acids N710, N711 and V721 from the AAV9 VP1 polypeptide interact with the PKD1 receptor.

E64. The AAV capsid polypeptide of any one of E28-E34 or E63, wherein any one or more of amino acids W504 and T505 from the AAV6 VP1 polypeptide and wherein any one or more of amino acids S263, S269, S386, Q387, D384, N270 from the AAV9 VP1 polypeptide interact with the PKD2 receptor.

E65. An AAV capsid polypeptide comprising or consisting of an amino acid sequence selected from the group consisting of SEQ ID NO:25, SEQ ID NO:27, SEQ ID NO:29, SEQ ID NO: 31, SEQ ID NO:33, SEQ ID NO:35, SEQ ID NO:37, SEQ ID NO:39, SEQ ID NO:41, SEQ ID NO: 43, SEQ ID NO:45 and SEQ ID NO:47.

E66. An AAV capsid polypeptide comprising or consisting of an amino acid sequence encoded by a nucleic acid sequence selected from the group consisting of SEQ ID NO:26, SEQ ID NO: 28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46 and SEQ ID NO:48.

E67. A recombinant adeno-associated viral (rAAV) vector comprising an AAV capsid polypeptide of any one of E1-E66.

E68. The rAAV vector of E67, further comprising a nucleic acid comprising a transgene.

E69. The rAAV vector of E68, wherein the transgene encodes a therapeutic protein or a reporter protein.

E70. The rAAV vector of E69, wherein the reporter protein is a green fluorescent protein (GFP) or enhanced green fluorescent protein (eGFP).

E71. The rAAV vector of any one of E67-E70, wherein the tropism of the vector differs from the tropism of an otherwise identical rAAV vector comprising an AAV capsid polypeptide of a parental serotype.

E72. The rAAV vector of E71, wherein the tropism of the vector for any one of the liver, heart, skeletal muscle or central nervous system (e.g., brain, spinal cord) is decreased as compared to an otherwise identical rAAV vector comprising an AAV capsid polypeptide of a parental serotype.

E73. The rAAV vector of E71, wherein the tropism of the vector for any one of the liver, heart, skeletal muscle or central nervous system (e.g., brain, spinal cord) is increased as compared to an otherwise identical rAAV vector comprising an AAV capsid polypeptide of a parental serotype.

E74. A nucleic acid encoding the AAV capsid polypeptide of any one of E1-E66.

E75. The nucleic acid of E74, wherein the nucleic acid is a plasmid.

E76. A host cell comprising a nucleic acid encoding the AAV capsid polypeptide of any one of E1-E66.

E77. A pharmaceutical composition comprising i) a rAAV vector comprising an AAV capsid polypeptide of any one of E1-E66 and a vector genome and ii) a pharmaceutically acceptable excipient.

E78. A pharmaceutical composition comprising i) a rAAV vector of any one of E67-E73 and ii) a pharmaceutically acceptable excipient.

E79. A method of making a rAAV vector, the method comprising i) transfecting a host cell in culture with a plasmid comprising a nucleic acid encoding an AAV capsid polypeptide of any one of E1-E66, and a plasmid comprising a nucleic acid encoding a therapeutic protein or a reporter protein and ii) isolating the rAAV vector from the host cell and/or from the culture media.

E80. The method of E79, wherein an amount of rAAV vector made by the host cell is substantially similar to an amount of rAAV vector made by an otherwise identical host cell comprising a nucleic acid encoding an AAV capsid polypeptide of a parental serotype.

E81. The method of E80, wherein an amount of rAAV vector made by the host cell is about 80% to 140% of an amount of rAAV vector made by an otherwise identical host cell comprising a nucleic acid encoding an AAV capsid polypeptide of a parental serotype.

E82. The method of any one of E79-E81, wherein the parental serotype is AAV9 or rh10.

E83. The method of any one of E79-E82, wherein the rAAV vector comprises an AAV capsid polypeptide comprising the amino acid sequence of any one of SEQ ID NO:25, SEQ ID NO:27, SEQ ID NO:29, SEQ ID NO:31, SEQ ID NO:33, SEQ ID NO:35, SEQ ID NO:37, SEQ ID NO:39 and SEQ ID NO:41.

E84. The method of E79, wherein an amount of rAAV vector made by the host cell is 5 to 100-fold greater than an amount of rAAV vector made by an otherwise identical host cell comprising a nucleic acid encoding an AAV capsid polypeptide of a parental serotype.

E85. The method of E84, wherein the parental serotype is AAV5.

E86. The method of E84 or E85, wherein the rAAV vector comprises an AAV capsid polypeptide comprising the amino acid sequence of SEQ ID NO:43, SEQ ID NO: 45 or SEQ ID NO: 47.

E87. A method of transducing a target cell, the method comprising contacting the target cell with a rAAV vector comprising an AAV capsid polypeptide of any one of E1-E66, a rAAV vector of any one of E67-E73 or a pharmaceutical composition of E77 or E78 such that the rAAV vector is introduced into the target cell.

E88. The method of E87, wherein the target cell is a liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells, hematopoietic (e.g., blood or lymph) or stem (e.g., pluripotent or multipotent progenitor) cell.

E89. The method of E87 or E88, wherein the target cell is an isolated cell and transduction occurs ex vivo.

E90. The method of E87 or E89, wherein the target cell is a cell within an organism and transduction occurs in vivo.

E91. The method of any one of E87-E90, wherein the cell that is transduced expresses a therapeutic protein or a reporter protein.

E92. The method of E91, wherein the reporter protein is green fluorescent protein (GFP) or enhanced green fluorescent protein (eGFP).

E93. The method of any one of E90-E92, wherein the transduced cell within an organism expresses the transgene encoding a therapeutic protein.

E94. A rAAV vector comprising an AAV capsid polypeptide of any one of E1-E66, a rAAV vector of E67-E73 or a pharmaceutical composition of E77 or E78 for use in treating and/or preventing a disease, disorder or condition.

E95. Use of a rAAV vector comprising an AAV capsid polypeptide of any one of E1-E66, a rAAV vector of E67-E73 or a pharmaceutical composition of E77 or E78 in the manufacture of a medicament for treating and/or preventing a disease, disorder or condition.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

The following Examples describe studies in which the inventors discovered that by substituting certain domains in a parental VP1 polypeptide (e.g., AAV9) with a homologous domain from another AAV serotype (e.g., AAV6) they were able to develop novel AAV capsids with novel tropism profiles. The inventors also discovered that these chimeric AAV capsids were useful for the production of rAAV vectors and that the rAAV vectors could be purified by standard methods for the production of vectors suitable for gene therapy applications.

### Example 1: Chimeric AAV Capsids with a GH Loop Substitution

To develop chimeric AAV capsids, crystal and cryoEM structures were used as a guide for large domain exchanges. Using parental AAV serotypes 5, 9 and rh10, the nucleic acids corresponding to the regions including β-sheet G (amino acids FX₁X₂X₃YX₄ where X₁ is E, Q or T, where X₂ is F, I or M, where X₃, is S, T or V and where X₄, is E, K, N, Q, S or T) through β-sheet H (X₁X₂X₃IKNT where X₁ is Q or M, X₂ is I or M, X₃F, L or M) (referred to as the GH loop) and β-sheet G through β-sheet I (TQTSTGQVX₁VX₂X₃EWEX₄X₅ where X₁ is A, S or T, X₂ is E or K, X₃ is I or M, X₄ is I or L, X₅ is K or Q) (referred to as the GI loop) were identified (Nam et al. (2007) J. Virology 81 (22): 12260-12271).

For AAV capsids with GH loop substitutions, three capsid serotypes were used as the parental serotype: AAV5, AAV9, and AAVrh10. Chimeric capsids with GH loop substitutions were made by substituting amino acids from the region between β-sheets G and H of a parental AAV VP1 (e.g., AAV9) with the amino acids from the same region of a VP1 polypeptide from an alternative AAV serotype (e.g., AAV6) (FIG. 1). To do this a GH loop fragment was cut from a plasmid comprising an AAV capsid sequence using, for example, BsiWI and AfeI. The fragment was integrated by standard ligation into a parental VP1 capsid sequence within a plasmid at BsiWI and AfeI sites. Successful sequence insertion was confirmed by BaeI digestion (present in the AAV9 GH loop region), MluI digestion (present in the AAV8 GH loop region), ScaI digestion (present in the AAV7 GH loop region) and BspEI digestion (present in the AAV6 GH loop). Plasmids containing the chimeric VP1 nucleotide sequences were sequence verified.

The cross-over region from the parental AAV VP1 sequence to the substituted region of the alternative AAV VP1 sequence was initiated at the DNA sequence region that corresponded with amino acids within, or downstream of, the β-sheet G region of the VP1 amino acid sequence and ended at the DNA sequence region that corresponded with amino acids within, or upstream of, the β-sheet H region, or β-sheet I region, of the alternative VP1 amino acid sequence (Table 1). The resulting chimeric AAV capsids exhibited advantageous properties, including a tropism profile different from the parental AAV capsid, production values consistent with that of parental AAV serotypes and characteristics that permitted purification using methods common to the parental AAV serotype.

TABLE 1

| Chimeric | | SEQ ID |
|---|---|---|
| capsid | β-sheet G (bold and italic) with cross-over regions (within box) | NO: |
| AAV5GI | 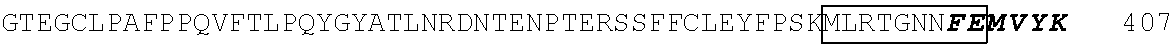 | 84 |
| bovine | FENVPFHSMYAHSQSLDRLMNPLLDQYLWELQSTTSGGTLNQGNSATNFAKLTKINFSGY 467 | |
| AAV5GI | 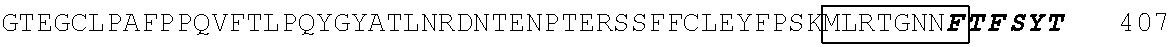 | 85 |
| porcine4 | FEDVPFHSSYAHSQSLDRLMNPLIDQYLYYLSKTAGGLAFSQ---------AGPTTMRNQ 458 | |
| AAV5GH | GTEGCLPAFPPQVFTLPQYGYATLNRDNTENPTERSSFFCLEYFPSKMLRTGNNFEFTYN 407 | 86 |
| porcine5 | 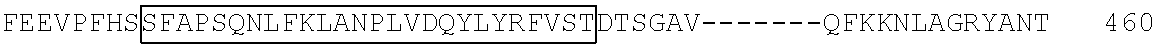 | |
| AAV9GH2 | AHEGCLPPFPADVFMIPQYGYLTLNDGS--QAVGRSSFYCLEYFPSQMLRTGNNFQFSYE 416 | 87 |
| | 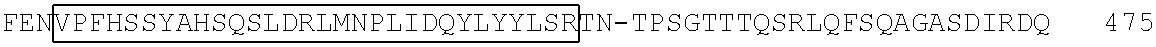 | |
| AAV9GH3B | AHEGCLPPFPADVFMIPQYGYLTLNDGS--QAVGRSSFYCLEYFPSQMLRTGNNFQFSYE 416 | 88 |
| | 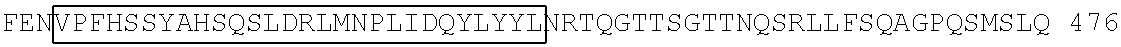 | |
| AAV9GH4 | AHEGCLPPFPADVFMIPQYGYLTLNDGS--QAVGRSSFYCLEYFPSQMLRTGNNFQFSYE 416 | 89 |
| | 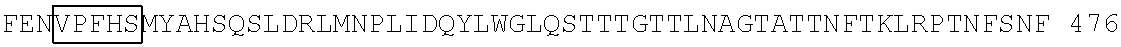 | |
| AAV9GH5 | AHEGCLPPFPADVFMIPQYGYLTLNDGS--QAVGRSSFYCLEYFPSQMLRTGNNFQFSYE 416 | 90 |
| | 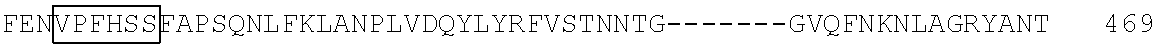 | |
| AAV9GH6 | 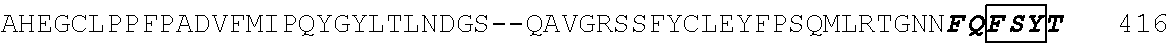 | 91 |
| | FEDVPFHSSYAHSQSLDRLMNPLIDQYLYYLNRTQNQ-SGSAQNKDLLFSRGSPAGMSVQ 475 | |
| AAV9GH7 | 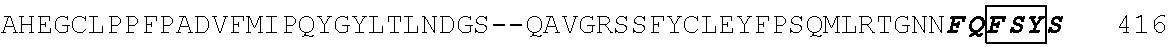 | 92 |
| | FEDVPFHSSYAHSQSLDRLMNPLIDQYLYYLARTQSNPGGTAGNRELQFYQGGPSTMAEQ 476 | |
| AAV9GH8 | 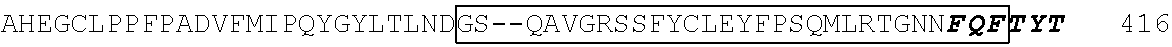 | 93 |
| | FEDVPFHSSYAHSQSLDRLMNPLIDQYLYYLSRTQT-TGGTANTQTLGFSQGGPNTMANQ 475 | |
| AAVrh10GH4 | AHQGCLPPFPADVFMIPQYGYLTLNNGS--QAVGRSSFYCLEYFPSQMLRTGNNFEFSYQ 417 | 94 |
| | 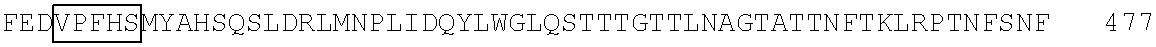 | |
| AAVrh10GH9 | AHQGCLPPFPADVFMIPQYGYLTLNNGS--QAVGRSSFYCLEYFPSQMLRTGNNFEFSYQ 417 | 95 |
| | 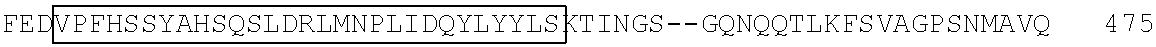 | |
| Chimeric | | SEQ ID |
| capsid | β-sheet H (bold and italic) with cross-over regions (within box) | NO: |
| AAV5GH | 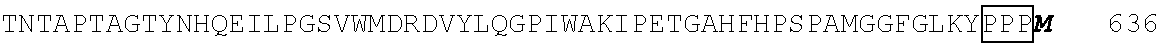 | 96 |
| porcine5 | 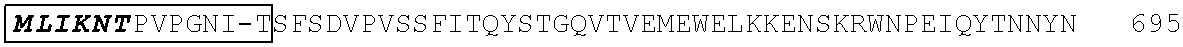 | |
| AAV9GH2 | 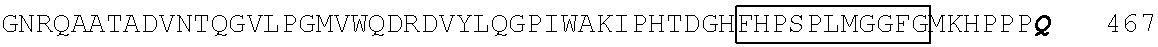 | 97 |
| | ILIKNTPVPADPPTAFNKDKLNSFITQYSTGQVSVEIEWELQKENSKRWNPEIQYTSNYY 707 | |
| AAV9GH3B | 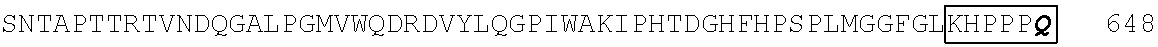 | 98 |
| | 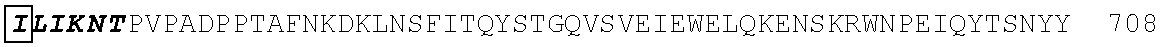 | |
| AAV9GH4 | 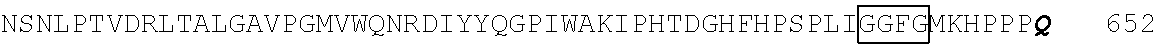 | 99 |
| | ILIKNTPVPADPPTAFNKDKLNSFITQYSTGQVSVEIEWELQKENSKRWNPEIQYTSNYY 712 | |
| AAV9GH5 | 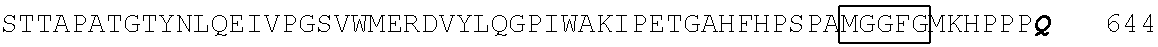 | 100 |
| | ILIKNTPVPADPPTAFNKDKLNSFITQYSTGQVSVEIEWELQKENSKRWNPEIQYTSNYY 704 | |
| AAV9GH6 | 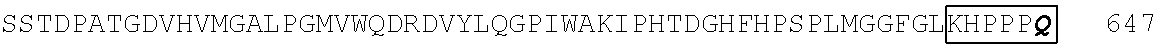 | 101 |
| | 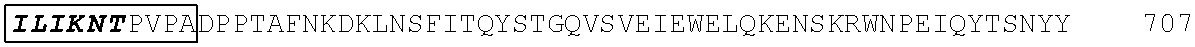 | |
| AAV9GH7 | 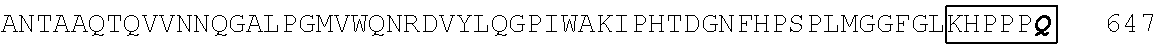 | 102 |
| | 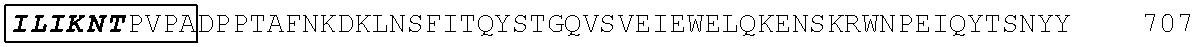 | |
| AAV9GH8 | 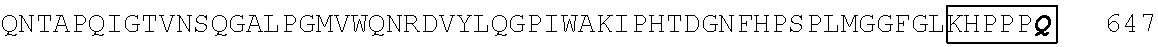 | 103 |
| | 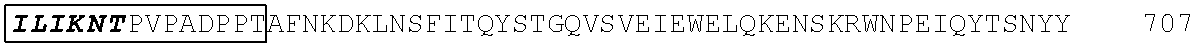 | |
| AAVrh10GH4 | 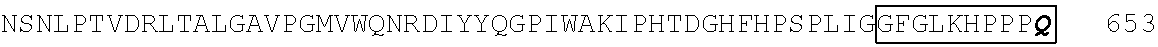 | 104 |
| | 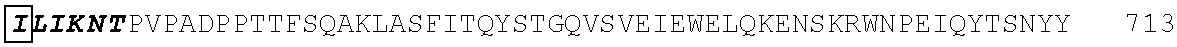 | |
| AAVrh10GH9 | 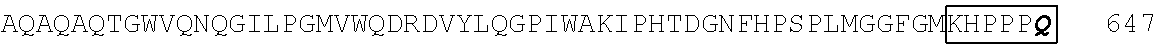 | 105 |
| | 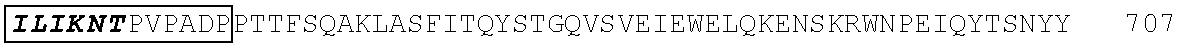 | |
| Chimeric | | SEQ ID |
| capsid | β-sheet I (bold and italic) with cross-over regions (within box) | NO: |
| AAV5GI | ATTVPTVDDVDGVGVYPGMVWQDRDIYYQGPIWAKIPHTDGHFHPSPLIGGFGLKSPPPQ 643 | 106 |
| bovine | 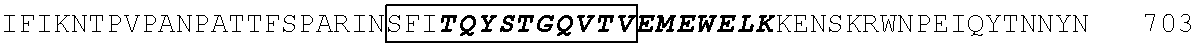 | |
| AAV5GI | SSTQATTAIVNAQGILPGMVWQDRDVYLQGPIWAKIPHTDGHFHPSPLMGGFGLKHPPPQ 630 | 107 |
| porcine4 | 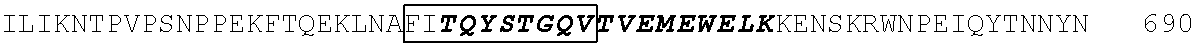 | |

Twelve novel capsids with GH loop or GI substitutions, were generated including: AAV9-GH-AAV2 (also referred to as AAV9GH2), AAV9-GH-AAV3B (also referred to as AAV9GH3B), AAV9-GH-AAV4 (also referred to as AAV9GH4), AAV9-GH-AAV5 (also referred to as AAV9GH5), AAV9-GH-AAV6 (also referred to as AAV9GH6), AAV9-GH-AAV7 (also referred to as AAV9GH7), AAV9-GH-AAV8 (also referred to as AAV9GH8), AAVrh10-GH-AAV9 (also referred to as AAVrh10GH9), AAVrh10-GH-AAV4 (also referred to as AAVrh10GH4), AAV5-GI-AAVporcine4 (also referred to as AAV5GIporcine4), AAV5-GH-AAVporcine5 (also referred to as AAV5GHporcine5) and AAV5-GI-AAVbovine (also referred to as AAV5GIbovine). Similarity, as measured by the percent distance (i.e., 1 minus the % identity), between the parental AAV9 VP1 capsid amino acid sequence and some of the VP1 amino acid sequences of the capsids with GH loop substitutions is shown in Table 2.

TABLE 2
Percent distance between capsids.

| | | | | | | |
|---|---|---|---|---|---|---|
| AAV9 | AAV9GH2 | AAV9GH6 | AAV9GH7 | AAV9GH4 | AAV9GHbov | AAV9GH5 |

| | | | | | | |
|---|---|---|---|---|---|---|
| AAV9 | 8 | 10 | 10 | 16 | 16 | 17 |
| AAV9GH2 | | 9 | 8 | 16 | 15 | 17 |
| AAV9GH6 | | | 9 | 16 | 15 | 18 |
| AAV9GH7 | | | | 16 | 16 | 18 |
| AAV9GH4 | | | | | 10 | 19 |
| AAV9GHbov | | | | | | 18 |
| AAV9GH5 | | | | | | |

Similarity, as measured by the percent conserved (i.e., the percentage of identical amino acids plus the percentage of similar amino acids), between the parental AAV9 VP1 polypeptide amino acid sequence and some of the VP1 amino acid sequences of the capsids with GH loop substitutions is shown in Table 3.

TABLE 3
Percent conserved between capsids

| | | | | | | |
|---|---|---|---|---|---|---|
| AAV9 | AAV9GH2 | AAV9GH6 | AAV9GH7 | AAV9GH4 | AAV9GHbov | AAV9GH5 |

| | | | | | | |
|---|---|---|---|---|---|---|
| AAV9 | 95 | 94 | 94 | 88 | 88 | 87 |
| AAV9GH2 | | 95 | 95 | 88 | 89 | 86 |
| AAV9GH6 | | | 96 | 88 | 90 | 86 |
| AAV9GH7 | | | | 88 | 88 | 86 |
| AAV9GH4 | | | | | 93 | 85 |
| AAV9GHbov | | | | | | 86 |
| AAV9GH5 | | | | | | |

rAAV vectors using the chimeric capsids were produced at 30 mL scale in a suspension HEK293 culture system to determine packaging capability and titer. Those chimeric capsids that yielded good titer, as compared to the parental capsid, were then produced at large capacity for in vivo testing using a bidirectional CMV driving enhanced GFP (eGFP) and firefly luciferase transgene.

rAAV vectors with an eGFP transgene were administered intravenously at a dose of 3E+13 vg/kg, or by biventricular (intracerebroventricular, “icv”) CNS administration at a dose of 2E+10 vg/ventricle, to Balb/c mice for biodistribution studies. The mice were injected with luciferin intravenously for bioluminescent imaging after 4 weeks.

C57BI/6 mice were also injected intravenously with 5E+12 vg/kg of each vector, and the animal tissues were harvested 4 weeks post-injection. Animal tissue was harvested and either frozen in liquid nitrogen for processing or fixed in formalin for immunofluorescent imaging. Tissues of interest were pulverized into a powder prior to DNA and RNA processing using Qiagen kits following manufacturer's protocol. Both DNA distribution and RNA expression levels were quantified by droplet digital PCR (ddPCR) using a GFP primer-probe set. Fixed tissue was paraffin embedded and sectioned. Tissue was then probed for GFP.

### Results

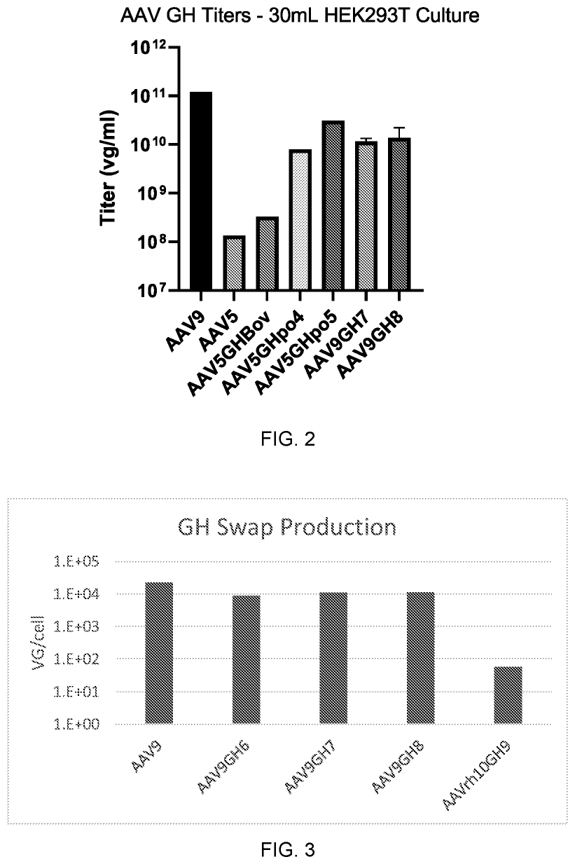

At the 30 mL production scale, vg/cell titer yields for the chimeric vectors comprising a capsid with a GH loop substitution were similar to AAV9 with the exception of AAV9GH3B, AAV9GH4, and AAV9GHrh10. The GH loop and GI loop substitutions into AAV5 yielded titers higher than AAV5 (FIG. 2). At the larger scale 2 liter suspension cultures, AAV9GH6, AAV9GH7, and AAV9GH8 yielded production titers similar to that of AAV9. AAVrh10GH9 titers were lower than those of AAV9 (FIG. 3).

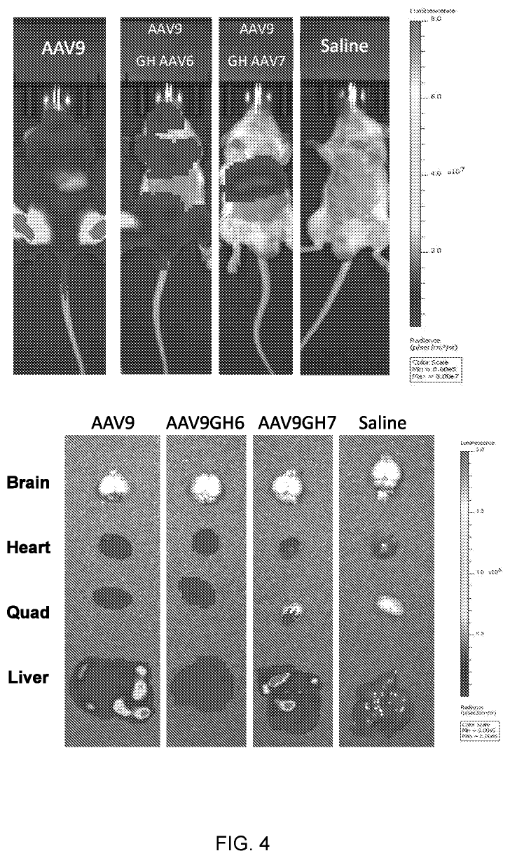

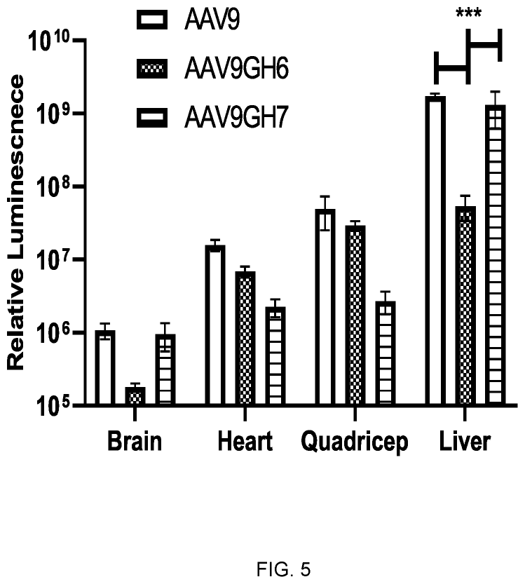

rAAV vectors with a capsid with a GH loop substitution demonstrated tissue tropism profiles that differed from the tropism profile of rAAV vectors comprising a parental AAV9 capsid. Based on the bioluminescent data, AAV9GH6 vectors administered intravenously demonstrated bioluminescence concentrated in the thoracic area and limbs of whole animals, indicative of tropism for the heart and skeletal muscle. Interestingly, there was reduced observation of bioluminescence in the liver region (FIG. 4). Ex vivo analysis of the tissues from mice injected intravenously with the AAV9GH6 vector, also demonstrated reduced bioluminescence in the liver as compared to the livers of mice injected with an AAV9 vector and an AAV9GH7 vector (FIG. 4). Quantification of the tissue bioluminescence from mice injected with AAV9, AAV9GH6 and AAV9GH7 vectors also demonstrated reduced biodistribution of the rAAV9GH6 vector in the liver and brain as compared to the parental rAAV9 vector and the rAAV9 with an AAV7 GH loop substitution (AAV9GH7) (FIG. 5). rAAV9GH8 vectors did not appear to target any tissue with specificity. Data were measured in photons/second and normalized to non-injected control mice.

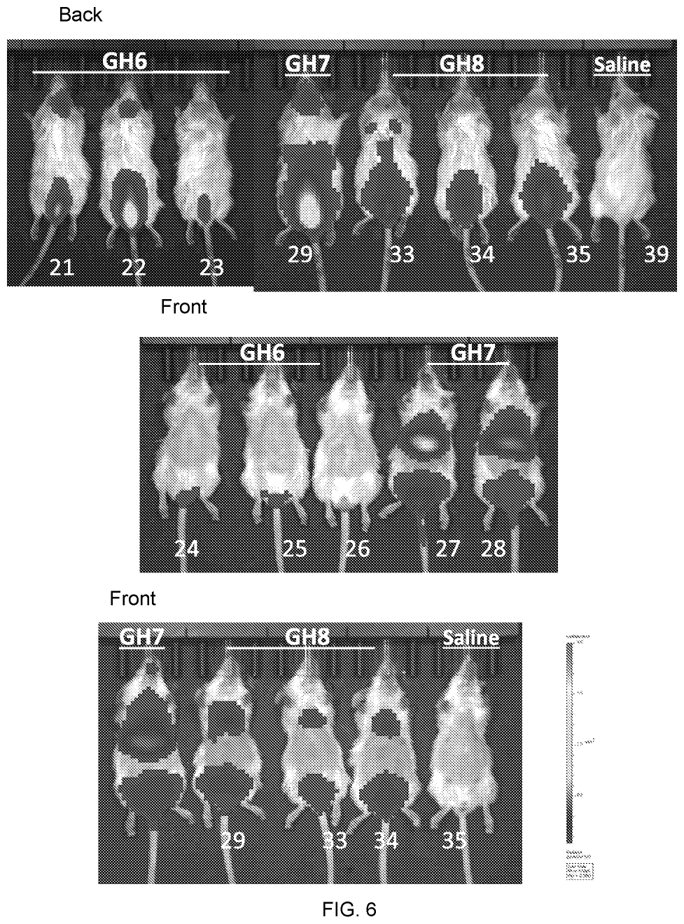

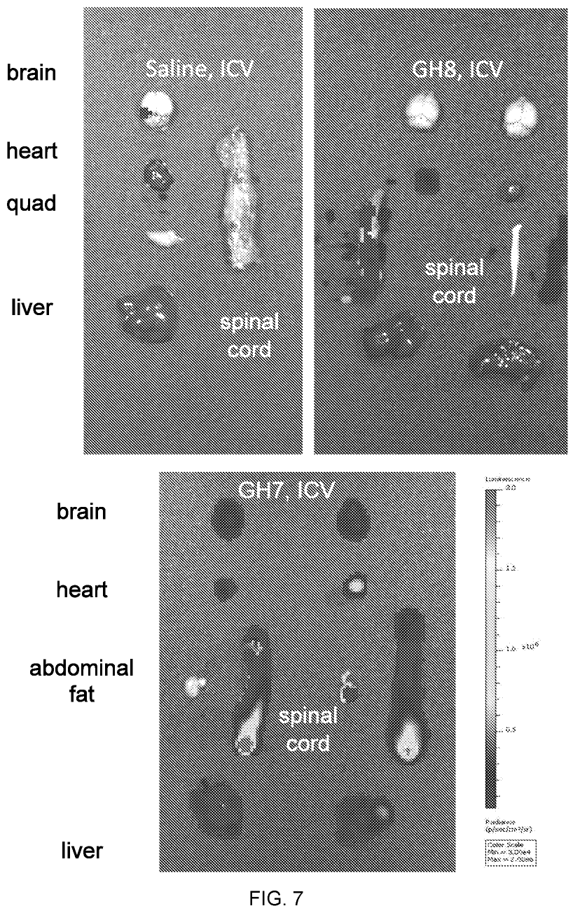

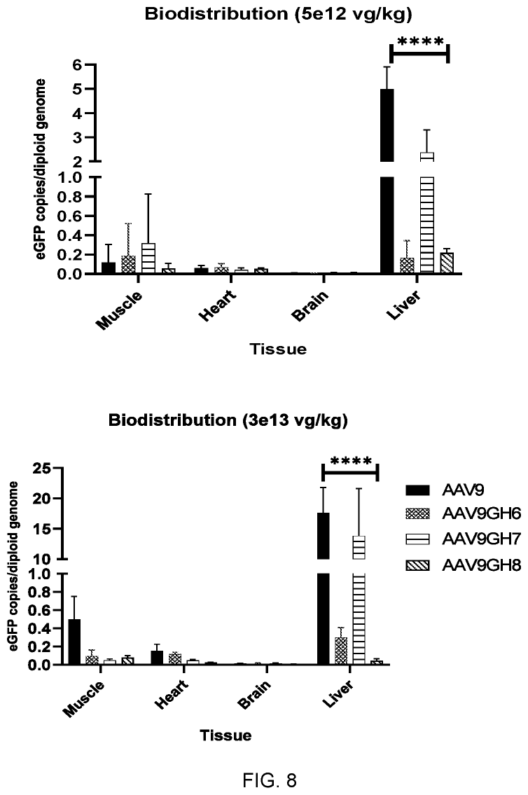

Administration of rAAV9GH7 vectors comprising an eGFP transgene by ICV injection resulted in significant levels of bioluminescent in the spine and brain as compared to mice administered AAV9GH8 and AAV9GH6 vectors by the same route. This was demonstrated by assessment of bioluminescence in both whole animal (FIG. 6) and ex vivo tissues (FIG. 7). Mice administered AAV9GH7 chimeric vectors also demonstrated significant bioluminescence in the liver and brain (Table 4). Data are measured in photons/second and normalized to non-injected control mice.

TABLE 4
Quantification of luminescence in ex vivo organs following
ICV administration of rAAV vectors with a GFP transgene.

| | | | | Quad | | |
|---|---|---|---|---|---|---|
| Mouse | AAV vector | Brain | Heart | muscle | Liver | Spine |
| 27 | AAV9GH7 | 2.42E+06 | 5.45E+05 | 3.30E+04 | 2.26E+07 | 2.79E+06 |
| 28 | AAV9GH7 | 3.29E+06 | 2.92E=06 | 2.89E+05 | 2.05E+07 | 5.76E+06 |
| 33 | AAV9GH8 | — | 6.37E+05 | — | — | 1.30E+06 |
| 34 | AAV8GH8 | — | — | — | — | 2.41E+04 |

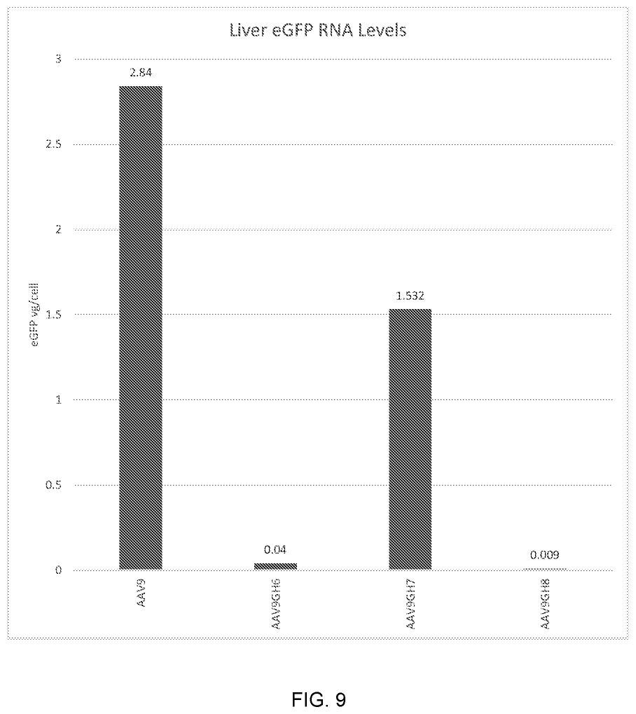

Biodistribution of the parental AAV9 and chimeric vectors administered IV was also determined by measuring the number of copies of expressed GFP (eGFP) per diploid genome in skeletal muscle, heart brain and liver tissue (FIG. 9). Significant differences were observed in the number of copies in the liver of mice administered the rAAV9 vector or the AAV9GH7 chimeric vector as compared to mice administered the chimeric rAAV9GH6 vector. This difference in biodistribution was observed in in mice administered 5E+12 vg/kg and in mice administered 3E+13 vg/kg. Also, mice administered the higher dose demonstrated an overall higher number of eGFP copies per diploid genome, representing a dose response. There was no significant difference in the number of eGFP copies per diploid genome in the heart among the vectors tested.

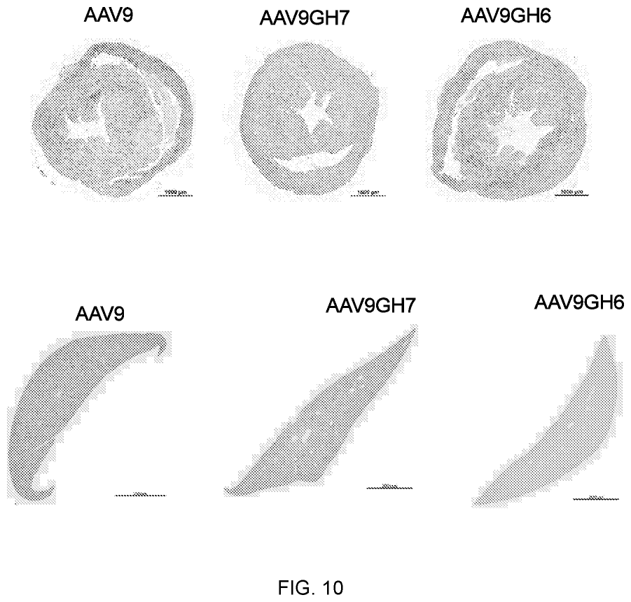

Expression of eGFP in the liver was measured in mice administered 3E+13 vg/kg by ddPCR (FIG. 10). Significant difference were observed in the expression levels as compared with the number of copies of eGFP measured per diploid genome. That is, RNA expression of eGFP in the liver was highest in the mice administered AAV9 or AAV9GH7 vector, and significantly lower in the liver of mice administered AAV9GH6 or AAV9GH8 vectors.

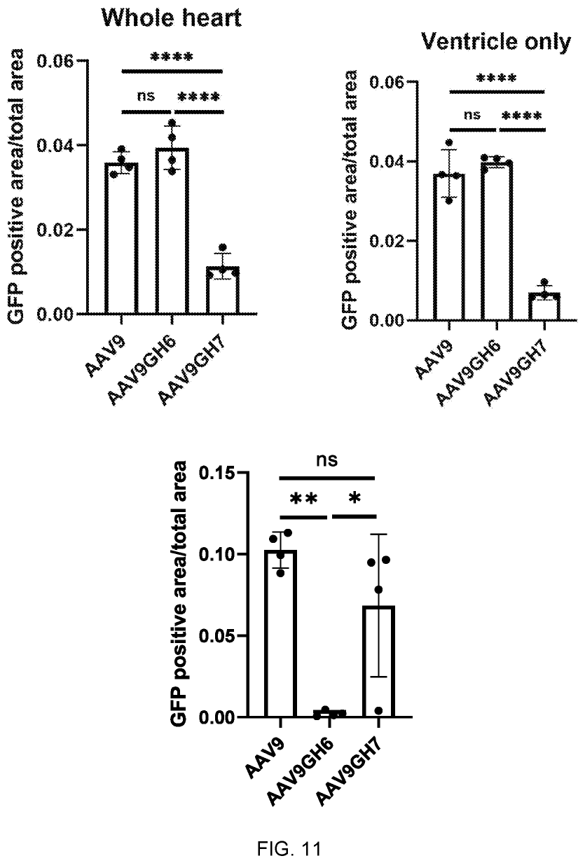

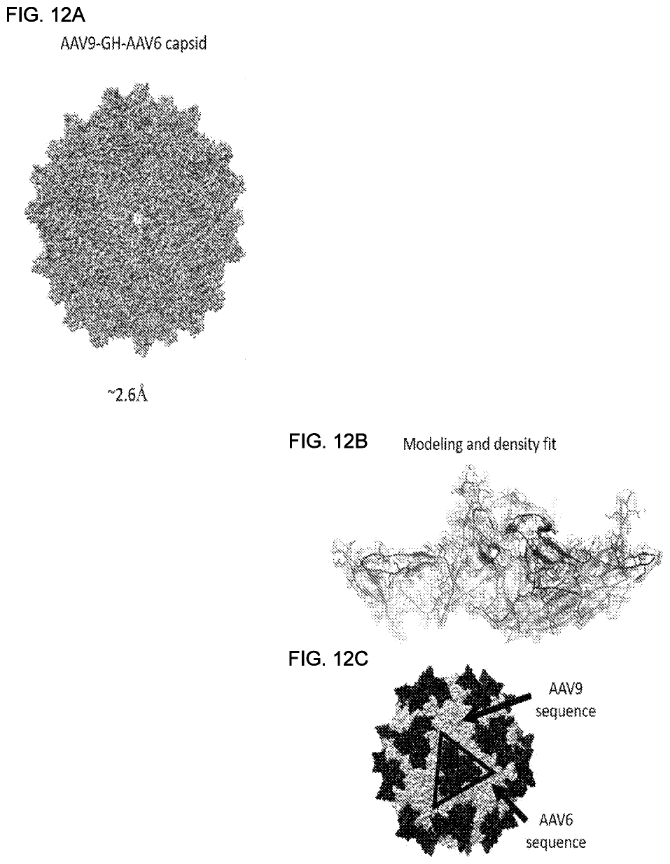

Immunohistochemistry (IHC) was performed on heart and liver tissues of mice administered 3E+13 vg/kg of rAAV9, rAAV9GH7 or rAAV9GH6 vector intravenously by staining against GFP protein (FIG. 11). The GFP positive area in the whole heart and in the ventricles only were quantified (FIG. 12). There was no significant difference in the GFP positive area in the whole heart or ventricle of mice administered the rAAV9 vector and the rAAV9GH6 vector. There was a significantly greater GFP positive area in the whole heart or ventricle of mice administered rAAV9 vector and mice administered rAAV9GH6 vector as compared to mice administered rAAV9GH7 vector (p<0.0001).

There was no significant difference in the GFP positive area in the liver of mice administered the rAAV9 vector and the rAAV9GH7 vector. There was a significantly greater GFP positive area in the liver of mice administered rAAV9 vector (p<0.01) and mice administered rAAV9GH7 vector (p<0.05) as compared to mice administered rAAV9GH6 vector (FIG. 12).

### Example 2: Cryo-Electron Microscopy (CryoEM) Analysis of AAV Capsids with a GH Loop Substitution

### Methods

Graphene oxide film-supported electron microscopy grids were prepared. AAV sample solutions were vitrified using a Vitrobot (ThermoFisher). The frozen grids were transferred to a FEI Titan Krios transmission electron microscope that operates at 300 kV. Target positions were set up in the SerialEM program, and high magnification (165KX) images were automatically collected with the program using a K2 direct detector camera (Gatan) using super resolution movie mode. The unbinned pixel size was 0.868 Å and the beam intensity was ˜8e/unbin pixel/s. The total electron dose on the sample for each movie was ˜40e/Å2. A total of 813 movies, each with 20 frames, was collected.

The cisTEM program (Grant et al. (2018) ELife 7: e353838) was used to process the data, including the steps of movie alignment, CTF correction, particle picking, 2D classification and auto refinement and post-processing. Following these steps, a ˜2.6 Å resolution electron density map was obtained. Based on these density maps and the known AAV9-GH-AAV6 amino acid sequence, atomic models were built with the Coot program (Emsley et al. (2010) Acta Crystallographica Section D-Biological Crystallography). The model was refined with the “Phenix.real-space-refine” tool (Afonine et al. (2018) Acta Crystallogr. D Struct. Biol.).

### Results

The AAV9-GH-AAV6 sample yielded a final 2.6A resolution structure map. The diameter of the capsid was ˜280 Å, which is similar to the diameter of capsids from other wild type AAV serotypes. The structure solution indicated that the AAV9-GH-AAV6 capsid was intact despite its non-natural sequence. The features from the EM density map supported the model building of the AAV9-GH-AAV6 capsid (FIG. 12A). Majority of the side chain configuration is well determined and fit into the density map well (FIG. 12B). On the capsid surface, the swapped sequence from the GH region are largely surface exposed and centered around the 3 fold axis (FIG. 12C).

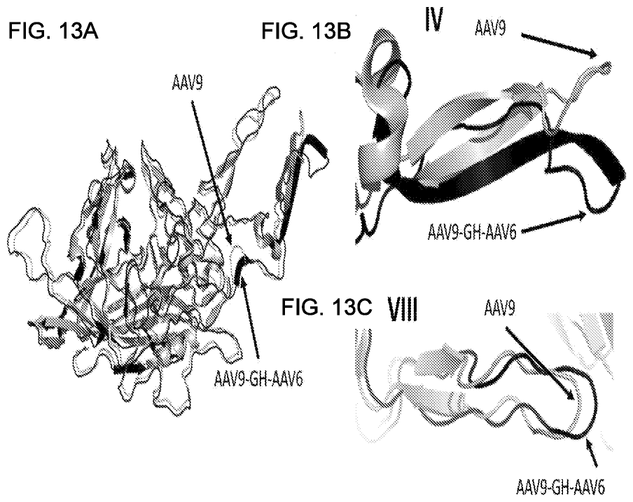

The structures from AAV9-GH-AAV6 and published AAV9 (PDB cod: 3UX1) were compared. The two showed similarity with an overall root-mean-square deviation (RMSD) of 1.275 Å (FIG. 13A). However, the difference was more pronounced in the loop IV and VIII regions (FIG. 13B and FIG. 13C).

The AAV9-GH-AAV6 structure was also compared with the published structure of AAV6 (PDB code: 3SHM). The overall RMSD between the two was 1.103 Å (FIG. 14A) and obvious local structural differences were also found in the loop IV and VIII regions (FIG. 14B and FIG. 14C). It is known that loop IV and VIII of the capsid are involved in interactions with cell surface glycans such as heparan sulfate proteoglycan (HSPG) and sialic acids (SIA) (Agbandje-McKenna and Kleinschmidt (2012) AAV Capsid Structure and Cell Interactions. In: Snyder R., Moullier P. (eds) Adeno-Associated Virus. Methods in Molecular Biology (Methods and Protocols), vol 807. Humana Press). The observation of overall structural similarity of the chimeric capsids with GH loop substitutions with wild type AAV serotype capsids indicates that the chimeric capsids are functional capsids and maintain the integrity of the virus. The observed differences in surface exposed loops indicates that variation in surface properties can contribute to the change in the tropism profile of the capsid.

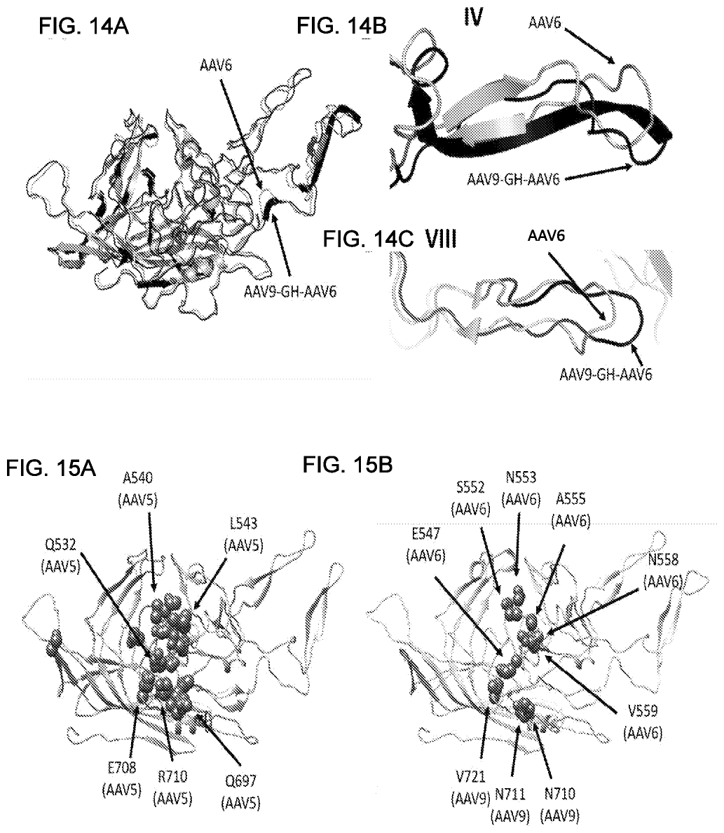

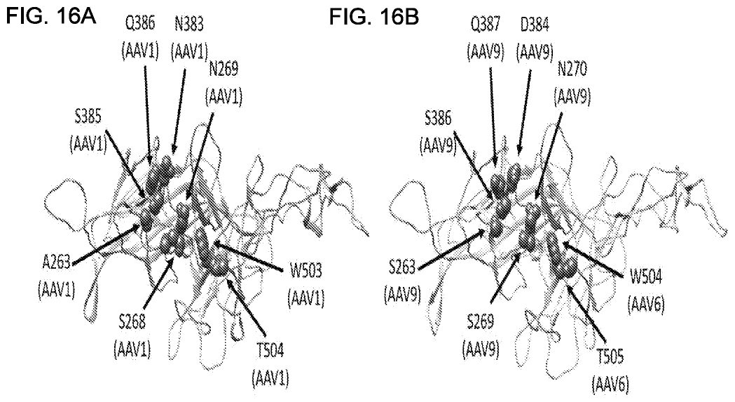

In addition to using cell surface glycans (including heparan sulfate proteoglycans, N-terminal galactose and sialic acid moieties) for initial engagement by an AAV virus, a glycoprotein that contains five polycystic kidney disease (PKD) repeat domains was recently identified as an AAV receptor. It has been shown that different subdomains of this receptor bind with AAV serotypes with different affinity. Thus, surface properties of an AAV capsid determines its interactions with cell surface receptors and may alter biodistribution. Using the known receptor binding regions of AAV5 with PKD1 and of AAV1 with PKD2 (Zhang et al. (2019) Nat. Comm. 10:3760) (FIG. 15A and FIG. 16A), the corresponding regions and residues of the AAV9-GH-AAV6 capsid acid sequence were identified (FIG. 15B and FIG. 16B). These corresponding regions are comprised of amino acids originating from the parental AAV9 VP1 sequence and from the AAV6 GH loop substitution sequence. Thus, interaction of the AAV9-GH-AAV6 capsid with AAV receptor(s) is likely mediated by regions and amino acid residues from both the AAV9 capsid sequence and the AAV6 capsid sequence, providing an explanation for altered biodistribution of chimeric capsids with GH or GI loop substitutions.

### EQUIVALENTS

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, textbooks, and the like, and the references cited therein, to the extent that they are not already, are hereby incorporated herein by reference in their entirety.

SEQUENCES

| SEQ | | |
|---|---|---|
| ID | | |
| NO: | Description | Sequence |
| 1 | AAV2 VP1 | maadgylpdwledtlsegirqwwklkpgppppkpaerhkddsrglvlpgykylgp |
| | amino acid | fngldkgepvneadaaalehdkaydrqldsgdnpylkynhadaefqerlkedtsf |
| | sequence | ggnlgravfqakkrvleplglveepvktapgkkrpvehspvepdsssgtgkagqq |
| | (GH loop | parkrlnfgqtgdadsvpdpqplgqppaapsglgtntmatgsgapmadnnegadg |
| | region | vgnssgnwhcdstwmgdrvittstrtwalptynnhlykqissqsgasndnhyfgy |
| | underlined) | stpwgyfdfnrfhchfsprdwqrlinnnwgfrpkrlnfklfniqvkevtqndgtt |
| | | tiannltstvqvftdseyqlpyvlgsahqgclppfpadvfmvpqygyltlnngsq |
| | | avgrssfycleyfpsqmlrtgnnftfsytfedvpfhssyahsqsldrlmnplidq |
| | | ylyylsrtntpsgtttqsrlqfsqagasdirdqsrnwlpgpcyrqqrvsktsadn |
| | | nnseyswtgatkyhlngrdslvnpgpamashkddeekffpqsgvlifgkqgsekt |
| | | nvdiekvmitdeeeirttnpvateqygsvstnlqrgnrqaatadvntqgvlpgmv |
| | | wqdrdvylqgpiwakiphtdghfhpsplmggfglkhpppqilikntpvpanpstt |
| | | fsaakfasfitqystgqvsveiewelqkenskrwnpeiqytsnynksvnvdftvd |
| | | tngvyseprpigtryltrnl |
| 2 | AAV2 VP1 | atggctgccgatggttatcttccagattggctcgaggacactctctctgaaggaa |
| | nucleotide | taagacagtggtggaagctcaaacctggcccaccaccaccaaagcccgcagagcg |
| | sequence | gcataaggacgacagcaggggtcttgtgcttcctgggtacaagtacctcggaccc |
| | | ttcaacggactcgacaagggagagccggtcaacgaggcagacgccgcggccctcg |
| | | agcacgacaaagcctacgaccggcagctcgacagcggagacaacccgtacctcaa |
| | | gtacaaccacgccgacgcggagtttcaggagcgccttaaagaagatacgtctttt |
| | | gggggcaacctcggacgagcagtcttccaggcgaaaaagagggttcttgaacctc |
| | | tgggcctggttgaggaacctgttaagacggctccgggaaaaaagaggccggtaga |
| | | gcactctcctgtggagccagactcctcctcgggaaccggaaaggcgggccagcag |
| | | cctgcaagaaaaagattgaattttggtcagactggagacgcagactcagtacctg |
| | | acccccagcctctcggacagccaccagcagccccctctggtctgggaactaatac |
| | | gatggctacaggcagtggcgcaccaatggcagacaataacgagggcgccgacgga |
| | | gtgggtaattcctcgggaaattggcattgcgattccacatggatgggcgacagag |
| | | tcatcaccaccagcacccgaacctgggccctgcccacctacaacaaccacctcta |
| | | caaacaaatttccagccaatcaggagcctcgaacgacaatcactactttggctac |
| | | agcaccccttgggggtattttgacttcaacagattccactgccacttttcaccac |
| | | gtgactggcaaagactcatcaacaacaactggggattccgacccaagagactcaa |
| | | cttcaagctctttaacattcaagtcaaagaggtcacgcagaatgacggtacgacg |
| | | acgattgccaataaccttaccagcacggttcaggtgtttactgactcggagtacc |
| | | agctcccgtacgtcctcggctcggcgcatcaaggatgcctcccgccgttoccagc |
| | | agacgtcttcatggtgccacagtatggatacctcaccctgaacaacgggagtcag |
| | | gcagtaggacgctcttcattttactgcctggagtactttccttctcagatgctgc |
| | | gtaccggaaacaactttaccttcagctacacttttgaggacgttcctttccacag |
| | | cagctacgctcacagccagagtctggaccgtctcatgaatcctctcatcgaccag |
| | | tacctgtattacttgagcagaacaaacactccaagtggaaccaccacgcagtcaa |
| | | ggcttcagttttctcaggccggagcgagtgacattcgggaccagtctaggaactg |
| | | gcttcctggaccctgttaccgccagcagcgagtatcaaagacatctgcggataac |
| | | aacaacagtgaatactcgtggactggagctaccaagtaccacctcaatggcagag |
| | | actctctggtgaatccgggcccggccatggcaagccacaaggacgatgaagaaaa |
| | | gttttttcctcagagcggggttctcatctttgggaagcaaggctcagagaaaaca |
| | | aatgtggacattgaaaaggtcatgattacagacgaagaggaaatcaggacaacca |
| | | atcccgtggctacggagcagtatggttctgtatctaccaacctccagagaggcaa |
| | | cagacaagcagctaccgcagatgtcaacacacaaggcgttcttccaggcatggtc |
| | | tggcaggacagagatgtgtaccttcaggggcccatctgggcaaagattccacaca |
| | | cggacggacattttcacccctctcccctcatgggtggattcggacttaaacaccc |
| | | tcctccacagattctcatcaagaacaccccggtacctgcgaatccttcgaccacc |
| | | ttcagtgcggcaaagtttgcttccttcatcacacagtactccacgggacaggtca |
| | | gcgtggagatcgagtgggagctgcagaaggaaaacagcaaacgctggaatcccga |
| | | aattcagtacacttccaactacaacaagtctgttaatgtggactttactgtggac |
| | | actaatggcgtgtattcagagcctcgccccattggcaccagatacctgactcgta |
| | | atctgtaa |
| 3 | AAV3B VP1 | maadgylpdwlednlsegirewwalkpgvpqpkanqqhqdnrrglvlpgykylgp |
| | amino acid | gngldkgepvneadaaalehdkaydqqlkagdnpylkynhadaefqerlqedtsf |
| | sequence (GH | ggnlgravfqakkrileplglveeaaktapgkkrpvdqspqepdsssgvgksgkq |
| | loop region | parkrlnfgqtgdsesvpdpqplgeppaaptslgsntmasgggapmadnnegadg |
| | underlined) | vgnssgnwhcdsqwlgdrvittstrtwalptynnhlykqissqsgasndnhyfgy |
| | | stpwgyfdfnrfhchfsprdwqrlinnnwgfrpkklsfklfniqvkevtqndgtt |
| | | tiannltstvqvftdseyqlpyvlgsahqgclppfpadvfmvpqygyltlnngsq |
| | | avgrssfycleyfpsqmlrtgnnfqfsytfedvpfhssyahsqsldrlmnplidq |
| | | ylyylnrtqgttsgttnqsrllfsqagpqsmslqarnwlpgpcyrqqrlsktand |
| | | nnnsnfpwtaaskyhlngrdslvnpgpamashkddeekffpmhgnlifgkegtta |
| | | snaeldnvmitdeeeirttnpvateqygtvannlqssntapttrtvndqgalpgm |
| | | vwqdrdvylqgpiwakiphtdghfhpsplmggfglkhpppqimikntpvpanppt |
| | | tfspakfasfitqystgqvsveiewelqkenskrwnpeiqytsnynksvnvdftv |
| | | dtngvyseprpigtryltrnl |
| 4 | AAV3B VP1 | atggctgccgatggttatcttccagattggctcgaggacaacctttctgaaggca |
| | nucleotide | ttcgtgagtggtgggctctgaaacctggagtccctcaacccaaagcgaaccaaca |
| | sequence | acaccaggacaaccgtcggggtcttgtgcttccgggttacaaatacctcggaccc |
| | | ggtaacggactcgacaaaggagagccggtcaacgaggcggacgcggcagccctcg |
| | | aacacgacaaagcttacgaccagcagctcaaggccggtgacaacccgtacctcaa |
| | | gtacaaccacgccgacgccgagtttcaggagcgtcttcaagaagatacgtctttt |
| | | gggggcaaccttggcagagcagtcttccaggccaaaaagaggatccttgagcctc |
| | | ttggtctggttgaggaagcagctaaaacggctcctggaaagaagaggcctgtaga |
| | | tcagtctcctcaggaaccggactcatcatctggtgttggcaaatcgggcaaacag |
| | | cctgccagaaaaagactaaatttcggtcagactggcgactcagagtcagtcccag |
| | | accctcaacctctcggagaaccaccagcagcccccacaagtttgggatctaatac |
| | | aatggcttcaggcggtggcgcaccaatggcagacaataacgagggtgccgatgga |
| | | gtgggtaattcctcaggaaattggcattgcgattcccaatggctgggcgacagag |
| | | tcatcaccaccagcaccagaacctgggccctgcccacttacaacaaccatctcta |
| | | caagcaaatctccagccaatcaggagcttcaaacgacaaccactactttggctac |
| | | agcaccccttgggggtattttgactttaacagattccactgccacttctcaccac |
| | | gtgactggcagcgactcattaacaacaactggggattccggcccaagaaactcag |
| | | cttcaagctcttcaacatccaagttaaagaggtcacgcagaacgatggcacgacg |
| | | actattgccaataaccttaccagcacggttcaagtgtttacggactcggagtatc |
| | | agctcccgtacgtgctcgggtcggcgcaccaaggctgtctcccgccgtttccagc |
| | | ggacgtcttcatggtccctcagtatggatacctcaccctgaacaacggaagtcaa |
| | | gcggtgggacgctcatccttttactgcctggagtacttcccttcgcagatgctaa |
| | | ggactggaaataacttccaattcagctataccttcgaggatgtaccttttcacag |
| | | cagctacgctcacagccagagtttggatcgcttgatgaatcctcttattgatcag |
| | | tatctgtactacctgaacagaacgcaaggaacaacctctggaacaaccaaccaat |
| | | cacggctgctttttagccaggctgggcctcagtctatgtctttgcaggccagaaa |
| | | ttggctacctgggccctgctaccggcaacagagactttcaaagactgctaacgac |
| | | aacaacaacagtaactttccttggacagcggccagcaaatatcatctcaatggcc |
| | | gcgactcgctggtgaatccaggaccagctatggccagtcacaaggacgatgaaga |
| | | aaaatttttccctatgcacggcaatctaatatttggcaaagaagggacaacggca |
| | | agtaacgcagaattagataatgtaatgattacggatgaagaagagattcgtacca |
| | | ccaatcctgtggcaacagagcagtatggaactgtggcaaataacttgcagagctc |
| | | aaatacagctcccacgactagaactgtcaatgatcagggggccttacctggcatg |
| | | gtgtggcaagatcgtgacgtgtaccttcaaggacctatctgggcaaagattcctc |
| | | acacggatggacactttcatccttctcctctgatgggaggctttggactgaaaca |
| | | tccgcctcctcaaatcatgatcaaaaatactccggtaccggcaaatcctccgacg |
| | | actttcagcccggccaagtttgcttcatttatcactcagtactccactggacagg |
| | | tcagcgtggaaattgagtgggagctacagaaagaaaacagcaaacgttggaatcc |
| | | agagattcagtacacttccaactacaacaagtctgttaatgtggactttactgta |
| | | gacactaatggtgtttatagtgaacctcgccctattggaacccggtatctcacac |
| | | gaaacttgtaa |
| 5 | AAV4 VP1 | maadgylpdwlednlsegvrewwalqpgapkpkanqqhqdnarglvlpgykylgp |
| | amino acid | gngldkgepvnaadaaalehdkaydqqlkagdnpylkynhadaefqqrlqgdtsf |
| | sequence (GH | ggnlgravfqakkrvleplglveqagetapgkkrpliespqqpdsstgigkkgkq |
| | loop region | pakkklvfedetgagdgppegstsgamsddsemraaaggaaveggqgadgvgnas |
| | underlined) | gdwhcdstwseghvtttstrtwvlptynnhlykrlgeslqsntyngfstpwgyfd |
| | | fnrfhchfsprdwqrlinnnwgmrpkamrvkifniqvkevttsngettvannlts |
| | | tvqifadssyelpyvmdagqegslppfpndvfmvpqygycglvtgntsqqqtdrn |
| | | afycleyfpsqmlrtgnnfeitysfekvpfhsmyahsqsldrlmnplidqylwgl |
| | | qstttgttlnagtattnftklrptnfsnfkknwlpgpsikqqgfsktanqnykip |
| | | atgsdslikyethstldgrwsaltpgppmatagpadskfsnsqlifagpkqngnt |
| | | atvpgtliftseeelaatnatdtdmwgnlpggdqsnsnlptvdrltalgavpgmv |
| | | wqnrdiyyqgpiwakiphtdghfhpspliggfglkhpppqifikntpvpanpatt |
| | | fsstpvnsfitqystgqvsvqidweiqkerskrwnpevqftsnygqqnsllwapd |
| | | aagkytepraigtrylthhl |
| 6 | AAV4 VP1 | atggctgctgacggttaccttccagattggctagaggacaacctctctgaaggcg |
| | nucleotide | ttcgagagtggtgggcgctgcaacctggagcccctaaacccaaggcaaatcaaca |
| | sequence | acatcaggacaacgctcggggtcttgtgcttccgggttacaaatacctcggaccc |
| | | ggcaacggactcgacaagggggaacccgtcaacgcagcggacgcggcagccctcg |
| | | agcacgacaaggcctacgaccagcagctcaaggccggtgacaacccctacctcaa |
| | | gtacaaccacgccgacgcggagttccagcagcggcttcagggcgacacatcgttt |
| | | gggggcaacctcggcagagcagtcttccaggccaaaaagagggttcttgaacctc |
| | | ttggtctggttgagcaagcgggtgagacggctcctggaaagaagagaccgttgat |
| | | tgaatccccccagcagcccgactcctccacgggtatcggcaaaaaaggcaagcag |
| | | ccggctaaaaagaagctcgttttcgaagacgaaactggagcaggcgacggacccc |
| | | ctgagggatcaacttccggagccatgtctgatgacagtgagatgcgtgcagcagc |
| | | tggcggagctgcagtcgagggcggacaaggtgccgatggagtgggtaatgcctcg |
| | | ggtgattggcattgcgattccacctggtctgagggccacgtcacgaccaccagca |
| | | ccagaacctgggtcttgcccacctacaacaaccacctctacaagcgactcggaga |
| | | gagcctgcagtccaacacctacaacggattctccaccccctggggatactttgac |
| | | ttcaaccgcttccactgccacttctcaccacgtgactggcagcgactcatcaaca |
| | | acaactggggcatgcgacccaaagccatgcgggtcaaaatcttcaacatccaggt |
| | | caaggaggtcacgacgtcgaacggcgagacaacggtggctaataaccttaccagc |
| | | acggttcagatctttgcggactcgtcgtacgaactgccgtacgtgatggatgcgg |
| | | gtcaagagggcagcctgcctccttttcccaacgacgtctttatggtgccccagta |
| | | cggctactgtggactggtgaccggcaacacttcgcagcaacagactgacagaaat |
| | | gccttctactgcctggagtactttccttcgcagatgctgcggactggcaacaact |
| | | ttgaaattacgtacagttttgagaaggtgcctttccactcgatgtacgcgcacag |
| | | ccagagcctggaccggctgatgaaccctctcatcgaccagtacctgtggggactg |
| | | caatcgaccaccaccggaaccaccctgaatgccgggactgccaccaccaacttta |
| | | ccaagctgcggcctaccaacttttccaactttaaaaagaactggctgcccgggcc |
| | | ttcaatcaagcagcagggcttctcaaagactgccaatcaaaactacaagatccct |
| | | gccaccgggtcagacagtctcatcaaatacgagacgcacagcactctggacggaa |
| | | gatggagtgccctgacccccggacctccaatggccacggctggacctgcggacag |
| | | caagttcagcaacagccagctcatctttgcggggcctaaacagaacggcaacacg |
| | | gccaccgtacccgggactctgatcttcacctctgaggaggagctggcagccacca |
| | | acgccaccgatacggacatgtggggcaacctacctggcggtgaccagagcaacag |
| | | caacctgccgaccgtggacagactgacagccttgggagccgtgcctggaatggtc |
| | | tggcaaaacagagacatttactaccagggtcccatttgggccaagattcctcata |
| | | ccgatggacactttcacccctcaccgctgattggtgggtttgggctgaaacaccc |
| | | gcctcctcaaatttttatcaagaacaccccggtacctgcgaatcctgcaacgacc |
| | | ttcagctctactccggtaaactccttcattactcagtacagcactggccaggtgt |
| | | cggtgcagattgactgggagatccagaaggageggtccaaacgctggaaccccga |
| | | ggtccagtttacctccaactacggacagcaaaactctctgttgtgggctcccgat |
| | | gcggctgggaaatacactgagcctagggctatcggtacccgctacctcacccacc |
| | | acctgtaa |
| 7 | AAV5 VP1 | msfvdhppdwleevgeglreflgleagppkpkpnqqhqdqarglvlpgynylgpg |
| | amino acid | ngldrgepvnradevarehdisyneqleagdnpylkynhadaefqekladdtsfg |
| | sequence (GH | gnlgkavfqakkrvlepfglveegaktaptgkriddhfpkrkkarteedskpsts |
| | loop region | sdaeagpsgsqqlqipaqpasslgadtmsaggggplgdnnqgadgvgnasgdwhc |
| | underlined) | dstwmgdrvvtkstrtwvlpsynnhqyreiksgsvdgsnanayfgystpwgyfdf |
| | | nrfhshwsprdwqrlinnywgfrprslrvkifniqvkevtvqdstttiannltst |
| | | vqvftdddyqlpyvvgngtegclpafppqvftlpqygyatlnrdntenpterssf |
| | | fcleyfpskmlrtgnnfeftynfeevpfhssfapsqnlfklanplvdqylyrfvs |
| | | tnntggvqfnknlagryantyknwfpgpmgrtqgwnlgsgvnrasvsafattnrm |
| | | elegasyqvppqpngmtnnlqgsntyalentmifnsqpanpgttatylegnmlit |
| | | sesetqpvnrvaynvggqmatnnqssttapatgtynlqeivpgsvwmerdvylqg |
| | | piwakipetgahfhpspamggfglkhpppmmlikntpvpgnitsfsdvpvssfit |
| | | qystgqvtvemewelkkenskrwnpeiqytnnyndpqfvdfapdstgeyrttrpi |
| | | gtryltrpl |
| 8 | AAV5 VP1 | atgtcttttgttgatcaccctccagattggttggaagaagttggtgaaggtcttc |
| | nucleotide | gcgagtttttgggccttgaagcgggcccaccgaaaccaaaacccaatcagcagca |
| | sequence | tcaagatcaagcccgtggtcttgtgctgcctggttataactatctcggacccgga |
| | | aacggtctcgatcgaggagagcctgtcaacagggcagacgaggtcgcgcgagagc |
| | | acgacatctcgtacaacgagcagcttgaggcgggagacaacccctacctcaagta |
| | | caaccacgcggacgccgagtttcaggagaagctcgccgacgacacatccttcggg |
| | | ggaaacctcggaaaggcagtctttcaggccaagaaaagggttctcgaaccttttg |
| | | gcctggttgaagagggtgctaagacggcccctaccggaaagcggatagacgacca |
| | | ctttccaaaaagaaagaaggctcggaccgaagaggactccaagccttccacctcg |
| | | tcagacgccgaagctggacccagcggatcccagcagctgcaaatcccagcccaac |
| | | cagcctcaagtttgggagctgatacaatgtctgcgggaggtggcggcccattggg |
| | | cgacaataaccaaggtgccgatggagtgggcaatgcctcgggagattggcattgc |
| | | gattccacgtggatgggggacagagtcgtcaccaagtccacccgaacctgggtgc |
| | | tgcccagctacaacaaccaccagtaccgagagatcaaaagcggctccgtcgacgg |
| | | aagcaacgccaacgcctactttggatacagcaccccctgggggtactttgacttt |
| | | aaccgcttccacagccactggagcccccgagactggcaaagactcatcaacaact |
| | | actggggcttcagaccccggtccctcagagtcaaaatcttcaacattcaagtcaa |
| | | agaggtcacggtgcaggactccaccaccaccatcgccaacaacctcacctccacc |
| | | gtccaagtgtttacggacgacgactaccagctgccctacgtcgtcggcaacggga |
| | | ccgagggatgcctgccggccttccctccgcaggtctttacgctgccgcagtacgg |
| | | ttacgcgacgctgaaccgcgacaacacagaaaatcccaccgagaggagcagcttc |
| | | ttctgcctagagtactttcccagcaagatgctgagaacgggcaacaactttgagt |
| | | ttacctacaactttgaggaggtgcccttccactccagcttcgctcccagtcagaa |
| | | cctgttcaagctggccaacccgctggtggaccagtacttgtaccgcttcgtgagc |
| | | acaaataacactggcggagtccagttcaacaagaacctggccgggagatacgcca |
| | | acacctacaaaaactggttcccggggcccatgggccgaacccagggctggaacct |
| | | gggctccggggtcaaccgcgccagtgtcagcgccttcgccacgaccaataggatg |
| | | gagctcgagggcgcgagttaccaggtgcccccgcagccgaacggcatgaccaaca |
| | | acctccagggcagcaacacctatgccctggagaacactatgatcttcaacagcca |
| | | gccggcgaacccgggcaccaccgccacgtacctcgagggcaacatgctcatcacc |
| | | agcgagagcgagacgcagccggtgaaccgcgtggcgtacaacgtcggcgggcaga |
| | | tggccaccaacaaccagagctccaccactgcccccgcgaccggcacgtacaacct |
| | | ccaggaaatcgtgcccggcagcgtgtggatggagagggacgtgtacctccaagga |
| | | cccatctgggccaagatcccagagacgggggcgcactttcacccctctccggcca |
| | | tgggcggattcggactcaaacacccaccgcccatgatgctcatcaagaacacgcc |
| | | tgtgcccggaaatatcaccagcttctcggacgtgcccgtcagcagcttcatcacc |
| | | cagtacagcaccgggcaggtcaccgtggagatggagtgggagctcaagaaggaaa |
| | | actccaagaggtggaacccagagatccagtacacaaacaactacaacgaccccca |
| | | gtttgtggactttgccccggacagcaccggggaatacagaagcaccagacctatc |
| | | ggaacccgataccttacccgacccctttaa |
| 9 | AAV6 VP1 | maadgylpdwlednlsegirewwdlkpgapkpkanqqkqddgrglvlpgykylgp |
| | amino acid | fngldkgepvnaadaaalehdkaydqqlkagdnpylrynhadaefqerlqedtsf |
| | sequence (GH | ggnlgravfqakkrvlepfglveegaktapgkkrpveqspqepdsssgigktgqq |
| | loop region | pakkrlnfgqtgdsesvpdpqplgeppatpaavgpttmasgggapmadnnegadg |
| | underlined) | vgnasgnwhcdstwlgdrvittstrtwalptynnhlykqissastgasndnhyfg |
| | | ystpwgyfdfnrfhchfsprdwqrlinnnwgfrpkrlnfklfniqvkevttndgv |
| | | ttiannltstvqvfsdseyqlpyvlgsahqgclppfpadvfmipqygyltlnngs |
| | | qavgrssfycleyfpsqmlrtgnnftfsytfedvpfhssyahsqsldrlmnplid |
| | | qylyylnrtqnqsgsaqnkdllfsrgspagmsvqpknwlpgpcyrqqrvsktktd |
| | | nnnsnftwtgaskynlngresiinpgtamashkddkdkffpmsgvmifgkesaga |
| | | sntaldnvmitdeeeikatnpvaterfgtvavnlqssstdpatgdvhvmgalpgm |
| | | vwqdrdvylqgpiwakiphtdghfhpsplmggfglkhpppqilikntpvpanppa |
| | | efsatkfasfitqystgqvsveiewelqkenskrwnpevqytsnyaksanvdftv |
| | | dnnglyteprpigtryltrpl |
| 10 | AAV6 VP1 | atggctgccgatggttatcttccagattggctcgaggacaacctctctgagggca |
| | nucleotide | ttcgcgagtggtgggacttgaaacctggagccccgaaacccaaagccaaccagca |
| | sequence | aaagcaggacgacggccggggtctggtgcttcctggctacaagtacctcggaccc |
| | | ttcaacggactcgacaagggggagcccgtcaacgcggcggatgcagcggccctcg |
| | | agcacgacaaggcctacgaccagcagctcaaagcgggtgacaatccgtacctgcg |
| | | gtataaccacgccgacgccgagtttcaggagcgtctgcaagaagatacgtctttt |
| | | gggggcaacctcgggcgagcagtcttccaggccaagaagagggttctcgaacctt |
| | | ttggtctggttgaggaaggtgctaagacggctcctggaaagaaacgtccggtaga |
| | | gcagtcgccacaagagccagactcctcctcgggcattggcaagacaggccagcag |
| | | cccgctaaaaagagactcaattttggtcagactggcgactcagagtcagtccccg |
| | | acccacaacctctcggagaacctccagcaacccccgctgctgtgggacctactac |
| | | aatggcttcaggcggtggcgcaccaatggcagacaataacgaaggcgccgacgga |
| | | gtgggtaatgcctcaggaaattggcattgcgattccacatggctgggcgacagag |
| | | tcatcaccaccagcacccgaacatgggccttgcccacctataacaaccacctcta |
| | | caagcaaatctccagtgcttcaacgggggccagcaacgacaaccactacttcggc |
| | | tacagcaccccctgggggtattttgatttcaacagattccactgccatttctcac |
| | | cacgtgactggcagcgactcatcaacaacaattggggattccggcccaagagact |
| | | caacttcaagctcttcaacatccaagtcaaggaggtcacgacgaatgatggcgtc |
| | | acgaccatcgctaataaccttaccagcacggttcaagtcttctcggactcggagt |
| | | accagttgccgtacgtcctcggctctgcgcaccagggctgcctccctccgttccc |
| | | ggcggacgtgttcatgattccgcagtacggctacctaacgctcaacaatggcagc |
| | | caggcagtgggacggtcatccttttactgcctggaatatttcccatcgcagatgc |
| | | tgagaacgggcaataactttaccttcagctacaccttcgaggacgtgcctttcca |
| | | cagcagctacgcgcacagccagagcctggaccggctgatgaatcctctcatcgac |
| | | cagtacctgtattacctgaacagaactcagaatcagtccggaagtgcccaaaaca |
| | | aggacttgctgtttagccgggggtctccagctggcatgtctgttcagcccaaaaa |
| | | ctggctacctggaccctgttaccggcagcagcgcgtttctaaaacaaaaacagac |
| | | aacaacaacagcaactttacctggactggtgcttcaaaatataaccttaatgggc |
| | | gtgaatctataatcaaccctggcactgctatggcctcacacaaagacgacaaaga |
| | | caagttctttcccatgagcggtgtcatgatttttggaaaggagagcgccggagct |
| | | tcaaacactgcattggacaatgtcatgatcacagacgaagaggaaatcaaagcca |
| | | ctaaccccgtggccaccgaaagatttgggactgtggcagtcaatctccagagcag |
| | | cagcacagaccctgcgaccggagatgtgcatgttatgggagccttacctggaatg |
| | | gtgtggcaagacagagacgtatacctgcagggtcctatttgggccaaaattcctc |
| | | acacggatggacactttcacccgtctcctctcatgggcggctttggacttaagca |
| | | cccgcctcctcagatcctcatcaaaaacacgcctgttcctgcgaatcctccggca |
| | | gagttttcggctacaaagtttgcttcattcatcacccagtattccacaggacaag |
| | | tgagcgtggagattgaatgggagctgcagaaagaaaacagcaaacgctggaatcc |
| | | cgaagtgcagtatacatctaactatgcaaaatctgccaacgttgatttcactgtg |
| | | gacaacaatggactttatactgagcctcgccccattggcacccgttacctcaccc |
| | | gtcccctgtaa |
| 11 | AAV7 VP1 | maadgylpdwlednlsegirewwdlkpgapkpkanqqkqdngrglvlpgykylgp |
| | amino acid | fngldkgepvnaadaaalehdkaydqqlkagdnpylrynhadaefqerlqedtsf |
| | sequence (GH | ggnlgravfqakkrvleplglveegaktapakkrpvepspqrspdsstgigkkgq |
| | loop region | qparkrlnfgqtgdsesvpdpqplgeppaapssvgsgtvaagggapmadnnegad |
| | underlined) | gvgnasgnwhcdstwlgdrvittstrtwalptynnhlykqissetagstndntyf |
| | | gystpwgyfdfnrfhchfsprdwqrlinnnwgfrpkklrfklfniqvkevttndg |
| | | vttiannltstiqvfsdseyqlpyvlgsahqgclppfpadvfmipqygyltlnng |
| | | sqsvgrssfycleyfpsqmlrtgnnfefsysfedvpfhssyahsqsldrlmnpli |
| | | dqylyylartqsnpggtagnrelqfyqggpstmaeqaknwlpgpcfrqqrvsktl |
| | | dannnsnfawtgatkyhlngrnslvnpgvamathkddedrffpssgvlifgktga |
| | | tnkttlenvlmtneeeirptnpvateeygivssnlqaantaaqtqvvnnqgalpg |
| | | mvwqnrdvylqgpiwakiphtdgnfhpsplmggfglkhpppqilikntpvpanpp |
| | | evftpakfasfitqystgqvsveiewelqkenskrwnpeiqytsnfekqtgvdfa |
| | | vdsqgvyseprpigtryltrnl |
| 12 | AAV7 VP1 | atggctgccgatggttatcttccagattggctcgaggacaacctctctgagggca |
| | nucleotide | ttcgcgagtggtgggacctgaaacctggagccccgaaacccaaagccaaccagca |
| | sequence | aaagcaggacaacggccggggtctggtgcttcctggctacaagtacctcggaccc |
| | | ttcaacggactcgacaagggggagcccgtcaacgcggcggacgcagcggccctcg |
| | | agcacgacaaggcctacgaccagcagctcaaagcgggtgacaatccgtacctgcg |
| | | gtataaccacgccgacgccgagtttcaggagcgtctgcaagaagatacgtcattt |
| | | gggggcaacctcgggcgagcagtcttccaggccaagaagcgggttctcgaacctc |
| | | tcggtctggttgaggaaggcgctaagacggctcctgcaaagaagagaccggtaga |
| | | gccgtcacctcagcgttcccccgactcctccacgggcatcggcaagaaaggccag |
| | | cagcccgccagaaagagactcaatttcggtcagactggcgactcagagtcagtcc |
| | | ccgaccctcaacctctcggagaacctccagcagcgccctctagtgtgggatctgg |
| | | tacagtggctgcaggcggtggcgcaccaatggcagacaataacgaaggtgccgac |
| | | ggagtgggtaatgcctcaggaaattggcattgcgattccacatggctgggcgaca |
| | | gagtcattaccaccagcacccgaacctgggccctgcccacctacaacaaccacct |
| | | ctacaagcaaatctccagtgaaactgcaggtagtaccaacgacaacacctacttc |
| | | ggctacagcaccccctgggggtattttgactttaacagattccactgccacttct |
| | | caccacgtgactggcagcgactcatcaacaacaactggggattccggcccaagaa |
| | | gctgcggttcaagctcttcaacatccaggtcaaggaggtcacgacgaatgacggc |
| | | gttacgaccatcgctaataaccttaccagcacgattcaggtattctcggactcgg |
| | | aataccagctgccgtacgtcctcggctctgcgcaccagggctgcctgcctccgtt |
| | | cccggcggacgtcttcatgattcctcagtacggctacctgactctcaacaatggc |
| | | agtcagtctgtgggacgttcctccttctactgcctggagtacttcccctctcaga |
| | | tgctgagaacgggcaacaactttgagttcagctacagcttcgaggacgtgccttt |
| | | ccacagcagctacgcacacagccagagcctggaccggctgatgaatcccctcatc |
| | | gaccagtacttgtactacctggccagaacacagagtaacccaggaggcacagctg |
| | | gcaatcgggaactgcagttttaccagggcgggccttcaactatggccgaacaagc |
| | | caagaattggttacctggaccttgcttccggcaacaaagagtctccaaaacgctg |
| | | gatcaaaacaacaacagcaactttgcttggactggtgccaccaaatatcacctga |
| | | acggcagaaactcgttggttaatcccggcgtcgccatggcaactcacaaggacga |
| | | cgaggaccgctttttcccatccagcggagtcctgatttttggaaaaactggagca |
| | | actaacaaaactacattggaaaatgtgttaatgacaaatgaagaagaaattcgtc |
| | | ctactaatcctgtagccacggaagaatacgggatagtcagcagcaacttacaagc |
| | | ggctaatactgcagcccagacacaagttgtcaacaaccagggagccttacctggc |
| | | atggtctggcagaaccgggacgtgtacctgcagggtcccatctgggccaagattc |
| | | ctcacacggatggcaactttcacccgtctcctttgatgggcggctttggacttaa |
| | | acatccgcctcctcagatcctgatcaagaacactcccgttcccgctaatcctccg |
| | | gaggtgtttactcctgccaagtttgcttcgttcatcacacagtacagcaccggac |
| | | aagtcagcgtggaaatcgagtgggagctgcagaaggaaaacagcaagcgctggaa |
| | | cccggagattcagtacacctccaactttgaaaagcagactggtgtggactttgcc |
| | | gttgacagccagggtgtttactctgagcctcgccctattggcactcgttacctca |
| | | cccgtaatctgtaa |
| 13 | AAV8 VP1 | maadgylpdwlednlsegirewwalkpgapkpkanqqkqddgrglvlpgykylgp |
| | amino acid | fngldkgepvnaadaaalehdkaydqqlqagdnpylrynhadaefqerlqedtsf |
| | sequence (GH | ggnlgravfqakkrvleplglveegaktapgkkrpvepspqrspdsstgigkkgq |
| | loop region | qparkrlnfgqtgdsesvpdpqplgeppaapsgvgpntmaagggapmadnnegad |
| | underlined) | gvgsssgnwhcdstwlgdrvittstrtwalptynnhlykqisngtsggatndnty |
| | | fgystpwgyfdfnrfhchfsprdwqrlinnnwgfrpkrlsfklfniqvkevtqne |
| | | gtktiannltstiqvftdseyqlpyvlgsahqgclppfpadvfmipqygyltlnn |
| | | gsqavgrssfycleyfpsqmlrtgnnfqftytfedvpfhssyahsqsldrlmnpl |
| | | idqylyylsrtqttggtantqtlgfsqggpntmanqaknwlpgpcyrqqrvsttt |
| | | gqnnnsnfawtagtkyhlngrnslanpgiamathkddeerffpsngilifgkqna |
| | | ardnadysdvmltseeeikttnpvateeygivadnlqqqntapqigtvnsqgalp |
| | | gmvwqnrdvylqgpiwakiphtdgnfhpsplmggfglkhpppqilikntpvpadp |
| | | pttfnqskinsfitqystgqvsveiewelqkenskrwnpeiqytsnyykstsvdf |
| | | avntegvyseprpigtryltrnl |
| 14 | AAV8 VP1 | atggctgccgatggttatcttccagattggctcgaggacaacctctctgagggca |
| | nucleotide | ttcgcgagtggtgggcgctgaaacctggagccccgaagcccaaagccaaccagca |
| | sequence | aaagcaggacgacggccggggtctggtgcttcctggctacaagtacctcggaccc |
| | | ttcaacggactcgacaagggggagcccgtcaacgcggcggacgcagcggccctcg |
| | | agcacgacaaggcctacgaccagcagctgcaggcgggtgacaatccgtacctgcg |
| | | gtataaccacgccgacgccgagtttcaggagcgtctgcaagaagatacgtctttt |
| | | gggggcaacctcgggcgagcagtcttccaggccaagaagcgggttctcgaacctc |
| | | tcggtctggttgaggaaggcgctaagacggctcctggaaagaagagaccggtaga |
| | | gccatcaccccagcgttctccagactcctctacgggcatcggcaagaaaggccaa |
| | | cagcccgccagaaaaagactcaattttggtcagactggcgactcagagtcagttc |
| | | cagaccctcaacctctcggagaacctccagcagcgccctctggtgtgggacctaa |
| | | tacaatggctgcaggcggtggcgcaccaatggcagacaataacgaaggcgccgac |
| | | ggagtgggtagttcctcgggaaattggcattgcgattccacatggctgggcgaca |
| | | gagtcatcaccaccagcacccgaacctgggccctgcccacctacaacaaccacct |
| | | ctacaagcaaatctccaacgggacatcgggaggagccaccaacgacaacacctac |
| | | ttcggctacagcaccccctgggggtattttgactttaacagattccactgccact |
| | | tttcaccacgtgactggcagcgactcatcaacaacaactggggattccggcccaa |
| | | gagactcagcttcaagctcttcaacatccaggtcaaggaggtcacgcagaatgaa |
| | | ggcaccaagaccatcgccaataacctcaccagcaccatccaggtgtttacggact |
| | | cggagtaccagctgccgtacgttctcggctctgcccaccagggctgcctgcctcc |
| | | gttcccggcggacgtgttcatgattccccagtacggctacctaacactcaacaac |
| | | ggtagtcaggccgtgggacgctcctccttctactgcctggaatactttccttcgc |
| | | agatgctgagaaccggcaacaacttccagtttacttacaccttcgaggacgtgcc |
| | | tttccacagcagctacgcccacagccagagcttggaccggctgatgaatcctctg |
| | | attgaccagtacctgtactacttgtctcggactcaaacaacaggaggcacggcaa |
| | | atacgcagactctgggcttcagccaaggtgggcctaatacaatggccaatcaggc |
| | | aaagaactggctgccaggaccctgttaccgccaacaacgcgtctcaacgacaacc |
| | | gggcaaaacaacaatagcaactttgcctggactgctgggaccaaataccatctga |
| | | atggaagaaattcattggctaatcctggcatcgctatggcaacacacaaagacga |
| | | cgaggagcgtttttttcccagtaacgggatcctgatttttggcaaacaaaatgct |
| | | gccagagacaatgcggattacagcgatgtcatgctcaccagcgaggaagaaatca |
| | | aaaccactaaccctgtggctacagaggaatacggtatcgtggcagataacttgca |
| | | gcagcaaaacacggctcctcaaattggaactgtcaacagccagggggccttaccc |
| | | ggtatggtctggcagaaccgggacgtgtacctgcagggtcccatctgggccaaga |
| | | ttcctcacacggacggcaacttccacccgtctccgctgatgggcggctttggcct |
| | | gaaacatcctccgcctcagatcctgatcaagaacacgcctgtacctgcggatcct |
| | | ccgaccaccttcaaccagtcaaagctgaactctttcatcacgcaatacagcaccg |
| | | gacaggtcagcgtggaaattgaatgggagctgcagaaggaaaacagcaagcgctg |
| | | gaaccccgagatccagtacacctccaactactacaaatctacaagtgtggacttt |
| | | gctgttaatacagaaggcgtgtactctgaaccccgccccattggcacccgttacc |
| | | tcacccgtaatctgtaa |
| 15 | AAV9 VP1 | maadgylpdwlednlsegirewwalkpgapqpkanqqhqdnarglvlpgykylgp |
| | amino acid | gngldkgepvnaadaaalehdkaydqqlkagdnpylkynhadaefqerlkedtsf |
| | sequence (GH | ggnlgravfqakkrlleplglveeaaktapgkkrpveqspqepdssagigksgaq |
| | loop region | pakkrlnfgqtgdtesvpdpqpigeppaapsgvgsltmasgggapvadnnegadg |
| | underlined) | vgsssgnwhcdsqwlgdrvittstrtwalptynnhlykqisnstsggssndnayf |
| | | gystpwgyfdfnrfhchfsprdwqrlinnnwgfrpkrlnfklfniqvkevtdnng |
| | | vktiannltstvqvftdsdyqlpyvlgsahegclppfpadvfmipqygyltlndg |
| | | sqavgrssfycleyfpsqmlrtgnnfqfsyefenvpfhssyahsqsldrlmnpli |
| | | dqylyylsktingsgqnqqtlkfsvagpsnmavqgrnyipgpsyrqqrvsttvtq |
| | | nnnsefawpgasswalngrnslmnpgpamashkegedrffplsgslifgkqgtgr |
| | | dnvdadkvmitneeeikttnpvatesygqvatnhqsaqaqaqtgwvqnqgilpgm |
| | | vwqdrdvylqgpiwakiphtdgnfhpsplmggfgmkhpppqilikntpvpadppt |
| | | afnkdklnsfitqystgqvsveiewelqkenskrwnpeiqytsnyyksnnvefav |
| | | ntegvyseprpigtryltrnl |
| 16 | AAV9 VP1 | atggctgccgatggttatcttccagattggctcgaggacaaccttagtgaaggaa |
| | nucleotide | ttcgcgagtggtgggctttgaaacctggagcccctcaacccaaggcaaatcaaca |
| | sequence | acatcaagacaacgctcgaggtcttgtgcttccgggttacaaataccttggaccc |
| | | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | ccatagcagctacgctcacagccaaagcctggaccgactaatgaatccactcatc |
| | | gaccaatacttgtactatctctcaaagactattaacggttctggacagaatcaac |
| | | aaacgctaaaattcagtgtggccggacccagcaacatggctgtccagggaagaaa |
| | | ctacatacctggacccagctaccgacaacaacgtgtctcaaccactgtgactcaa |
| | | aacaacaacagcgaatttgcttggcctggagcttcttcttgggctctcaatggac |
| | | gtaatagcttgatgaatcctggacctgctatggccagccacaaagaaggagagga |
| | | ccgtttctttcctttgtctggatctttaatttttggcaaacaaggaactggaaga |
| | | gacaacgtggatgcggacaaagtcatgataaccaacgaagaagaaattaaaacta |
| | | ctaacccggtagcaacggagtcctatggacaagtggccacaaaccaccagagtgc |
| | | ccaagcacaggcgcagaccggctgggttcaaaaccaaggaatacttccgggtatg |
| | | gtttggcaggacagagatgtgtacctgcaaggacccatttgggccaaaattcctc |
| | | acacggacggcaactttcacccttctccgctgatgggagggtttggaatgaagca |
| | | cccgcctcctcagatcctcatcaaaaacacacctgtacctgcggatcctccaacg |
| | | gccttcaacaaggacaagctgaactctttcatcacccagtattctactggccaag |
| | | tcagcgtggagatcgagtgggagctgcagaaggaaaacagcaagcgctggaaccc |
| | | ggagatccagtacacttccaactattacaagtctaataatgttgaatttgctgtt |
| | | aatactgaaggtgtatatagtgaaccccgccccattggcaccagatacctgactc |
| | | gtaatctgtaa |
| 17 | AAVrh10 VP1 | maadgylpdwlednlsegirewwdlkpgapkpkanqqkqddgrglvlpgykylgp |
| | amino acid | fngldkgepvnaadaaalehdkaydqqlkagdnpylrynhadaefqerlqedtsf |
| | sequence (GH | ggnlgravfqakkrvleplglveegaktapgkkrpvepspqrspdsstgigkkgq |
| | loop region | qpakkrlnfgqtgdsesvpdpqpigeppagpsglgsgtmaagggapmadnnegad |
| | underlined) | gvgsssgnwhcdstwlgdrvittstrtwalptynnhlykqisngtsggstndnty |
| | | fgystpwgyfdfnrfhchfsprdwqrlinnnwgfrpkrlnfklfniqvkevtqne |
| | | gtktiannltstiqvftdseyqlpyvlgsahqgclppfpadvfmipqygyltlnn |
| | | gsqavgrssfycleyfpsqmlrtgnnfefsyqfedvpfhssyahsqsldrlmnpl |
| | | idqylyylsrtqstggtagtqqllfsqagpnnmsaqaknwlpgpcyrqqrvsttl |
| | | sqnnnsnfawtgatkyhlngrdslvnpgvamathkddeerffpssgvlmfgkqga |
| | | gkdnvdyssvmltseeeikttnpvateqygvvadnlqqqnaapivgavnsqgalp |
| | | gmvwqnrdvylqgpiwakiphtdgnfhpsplmggfglkhpppqilikntpvpadp |
| | | pttfsqaklasfitqystgqvsveiewelqkenskrwnpeiqytsnyykstnvdf |
| | | avntdgtyseprpigtryltrnl |
| 18 | AAVrh10 VP1 | atggctgccgatggttatcttccagattggctcgaggacaacctctctgagggca |
| | nucleotide | ttcgcgagtggtgggacttgaaacctggagccccgaaacccaaagccaaccagca |
| | sequence | aaagcaggacgacggccggggtctggtgcttcctggctacaagtacctcggaccc |
| | | ttcaacggactcgacaagggggagcccgtcaacgcggcggacgcagcggccctcg |
| | | agcacgacaaggcctacgaccagcagctcaaagcgggtgacaatccgtacctgcg |
| | | gtataaccacgccgacgccgagtttcaggagcgtctgcaagaagatacgtctttt |
| | | gggggcaacctcgggcgagcagtcttccaggccaagaagcgggttctcgaacctc |
| | | tcggtctggttgaggaaggcgctaagacggctcctggaaagaagagaccggtaga |
| | | gccatcaccccagcgttctccagactcctctacgggcatcggcaagaaaggccag |
| | | cagcccgcgaaaaagagactcaactttgggcagactggcgactcagagtcagtgc |
| | | ccgaccctcaaccaatcggagaaccccccgcaggcccctctggtctgggatctgg |
| | | tacaatggctgcaggcggtggcgctccaatggcagacaataacgaaggcgccgac |
| | | ggagtgggtagttcctcaggaaattggcattgcgattccacatggctgggcgaca |
| | | gagtcatcaccaccagcacccgaacctgggccctccccacctacaacaaccacct |
| | | ctacaagcaaatctccaacgggacttcgggaggaagcaccaacgacaacacctac |
| | | ttcggctacagcaccccctgggggtattttgactttaacagattccactgccact |
| | | tctcaccacgtgactggcagcgactcatcaacaacaactggggattccggcccaa |
| | | gagactcaacttcaagctcttcaacatccaggtcaaggaggtcacgcagaatgaa |
| | | ggcaccaagaccatcgccaataaccttaccagcacgattcaggtctttacggact |
| | | cggaataccagctcccgtacgtcctcggctctgcgcaccagggctgcctgcctcc |
| | | gttcccggcggacgtcttcatgattcctcagtacgggtacctgactctgaacaat |
| | | ggcagtcaggccgtgggccgttcctccttctactgcctggagtactttccttctc |
| | | aaatgctgagaacgggcaacaactttgagttcagctaccagtttgaggacgtgcc |
| | | ttttcacagcagctacgcgcacagccaaagcctggaccggctgatgaaccccctc |
| | | atcgaccagtacctgtactacctgtctcggactcagtccacgggaggtaccgcag |
| | | gaactcagcagttgctattttctcaggccgggcctaataacatgtcggctcaggc |
| | | caaaaactggctacccgggccctgctaccggcagcaacgcgtctccacgacactg |
| | | tcgcaaaataacaacagcaactttgcctggaccggtgccaccaagtatcatctga |
| | | atggcagagactctctggtaaatcccggtgtcgctatggcaacccacaaggacga |
| | | cgaagagcgattttttccgtccagcggagtcttaatgtttgggaaacagggagct |
| | | ggaaaagacaacgtggactatagcagcgttatgctaaccagtgaggaagaaatta |
| | | aaaccaccaacccagtggccacagaacagtacggcgtggtggccgataacctgca |
| | | acagcaaaacgccgctcctattgtaggggccgtcaacagtcaaggagccttacct |
| | | ggcatggtctggcagaaccgggacgtgtacctgcagggtcctatctgggccaaga |
| | | ttcctcacacggacggaaactttcatccctcgccgctgatgggaggctttggact |
| | | gaaacacccgcctcctcagatcctgattaagaatacacctgttcccgcggatcct |
| | | ccaactaccttcagtcaagctaagctggcgtcgttcatcacgcagtacagcaccg |
| | | gacaggtcagcgtggaaattgaatgggagctgcagaaagaaaacagcaaacgctg |
| | | gaacccagagattcaatacacttccaactactacaaatctacaaatgtggacttt |
| | | gctgttaacacagatggcacttattctgagcctcgccccatcggcacccgttacc |
| | | tcacccgtaatctgtaattgcttgttaatcaataaaccggttgattcgtttcagt |
| | | tgaactttggtctctgcgaagggcgaattcgtttaaacctgcaggactagaggtc |
| | | ctgtattagaggtcacgtgagtgttttgcgacattttgcgacaccatgtggtcac |
| | | gctgggtatttaagcccgagtgagcacgcagggtctccattttgaagcgggaggt |
| | | ttgaacgcgcagccgccaagccgaattctgcagatatccatcacactggcggccg |
| | | ctcgactag |
| 19 | Bovine AAV | msfvdhppdwlesigdgfreflgleagppkpkanqqkqdnarglvlpgykylgpg |
| | VP1 amino | ngldkgdpvnfadevarehdlsyqkqleagdnpylkynhadaefqeklasdtsfg |
| | acid | gnlgkavfqakkrileplglvetpdktapaakkrpleqspqepdsssgvgkkgkq |
| | sequence (GH | parkrlnfddepgagdgpppegpssgamstetemraaaggnggdagqgaegvgna |
| | loop region | sgdwhcdstwseshvtttstrtwvlptynnhlylrlgssnasdtfngfstpwgyf |
| | underlined) | dfnrfhchfsprdwqrlinnhwglrpksmqvrifniqvkevttsngettvsnnlt |
| | | stvqifadstyelpyvmdagqegslppfpndvfmvpqygycglvtggssqnqtdr |
| | | nafycleyfpsqmlrtgnnfemvykfenvpfhsmyahsqsldrlmnplldqylwe |
| | | lqsttsggtlnqgnsatnfakltktnfsgyrknwlpgpmmkqqrfsktasqnyki |
| | | pqgrnnsllhyetrttldgrwsnfapgtamataandatdfsqaqlifagpnitgn |
| | | tttdannlmftsedelratnprdtdlfghlatnqqnattvptvddvdgvgvypgm |
| | | vwqdrdiyyqgpiwakiphtdghfhpspliggfglkspppqifikntpvpanpat |
| | | tfsparinsfitqystgqvavkieweiqkerskrwnpevqftsnygaqdsllwap |
| | | dnagaykepraigsryltnhl |
| 20 | Bovine AAV | atgtcttttgttgaccaccctccagattggttggaatcgatcggcgacggctttc |
| | VP1 | gtgaatttctcggccttgaggcgggtcccccgaaacccaaggccaatcaacagaa |
| | nucleotide | gcaagataacgctcgaggtcttgtgcttcctgggtacaagtatcttggtcctggg |
| | sequence | aacggccttgataagggcgatcctgtcaattttgctgacgaggttgcccgagagc |
| | | acgacctctcctaccagaaacagcttgaggcgggcgataacccttacctcaagta |
| | | caaccacgcggacgcagagtttcaggagaaactcgcttctgacacttcttttggg |
| | | ggaaaccttgggaaggctgttttccaggctaaaaagaggattctcgaacctcttg |
| | | gcctggttgagacgccggataaaacggcgcctgcggcaaaaaagaggcctctaga |
| | | gcagagtcctcaagagccagactcctcgagcggagttggcaagaaaggcaaacag |
| | | cctgccagaaagagactcaactttgacgacgaacctggagccggagacgggcctc |
| | | ccccagaaggaccatcttccggagctatgtctactgagactgaaatgcgtgcagc |
| | | agctggcggaaatggtggcgatgcgggacaaggtgccgagggagtgggtaatgcc |
| | | tccggtgattggcattgcgattccacttggtcagagagccacgtcaccaccacct |
| | | caacccgcacctgggtcctgccgacctacaacaaccacctgtacctgcggctcgg |
| | | ctcgagcaacgccagcgacaccttcaacggattctccaccccctggggatacttt |
| | | gactttaaccgcttccactgccacttctcgccaagagactggcaaaggctcatca |
| | | acaaccactggggactgcgccccaaaagcatgcaagtccgcatcttcaacatcca |
| | | agttaaggaggtcacgacgtctaacggggagacgaccgtatccaacaacctcacc |
| | | agcacggtccagatctttgcggacagcacgtacgagctcccgtacgtgatggatg |
| | | caggtcaggagggcagcttgcctcctttccccaacgacgtgttcatggtgcctca |
| | | gtacgggtactgcggactggtaaccggaggcagctctcaaaaccagacagacaga |
| | | aatgccttctactgtctggagtactttcccagccagatgctgagaaccggaaaca |
| | | actttgagatggtgtacaagtttgaaaacgtgcccttccactccatgtacgctca |
| | | cagccagagcctggataggctgatgaacccgctgctggaccagtacctgtgggag |
| | | ctccagtctaccacctctggaggaactctcaaccagggcaattcagccaccaact |
| | | ttgccaagctgaccaaaacaaacttttctggctaccgcaaaaactggctcccggg |
| | | gcccatgatgaagcagcagagattctccaagactgccagtcaaaactacaagatt |
| | | ccccagggaagaaacaacagtctgctccattatgagaccagaactaccctcgacg |
| | | gaagatggagcaattttgccccgggaacggccatggcaaccgcagccaacgacgc |
| | | caccgacttctctcaggcccagctcatctttgcggggcccaacatcaccggcaac |
| | | accaccacagatgccaataacctgatgttcacttcagaagatgaacttagggcca |
| | | ccaacccccgggacactgacctgtttggccacctggcaaccaaccagcaaaacgc |
| | | caccaccgttcctaccgtagacgacgtggacggagtcggcgtgtacccgggaatg |
| | | gtgtggcaggacagagacatttactaccaagggcccatttgggccaaaattccac |
| | | acacggatggacactttcacccgtctcctctcattggcggatttggactgaaaag |
| | | cccgcctccacaaatattcatcaaaaacactcctgtacccgccaatcccgcaacg |
| | | accttctctccggccagaatcaacagcttcatcacccagtacagcaccggacagg |
| | | tggctgtcaaaatagaatgggaaatccagaaggagcggtccaagagatggaaccc |
| | | agaggtccagttcacgtccaactacggagcacaggactcgcttctctgggctccc |
| | | gacaacgccggagcctacaaagagcccagggccattggatcccgatacctcacca |
| | | accacctctag |
| 21 | Porcine4 AAV | msfvdhppdwleevgeglhefleleagppkpkpnqqkqdnarglvlpgynylgpf |
| | VP1 amino | ngldkgepvnradavarehdisyneqlqagdnpylkynhadaefqeklkddtsfg |
| | acid | gnlgkaifqakkrvlepfglveapvktapakkrpiekspaepssskgigkagqqp |
| | sequence (GH | arkrlnfgqtgdtdsaadpqplgeppaapsglgtgtmasgggapmadnnegadgv |
| | loop region | gnasgnwhcdstwlgdrvittstrtwalptynnhlykqisssnyganndnhyfgy |
| | underlined) | stpwgyfdfnrfhchfsprdwqrlinnnwgfrpkrlnfklfniqvkevtetdgtk |
| | | tiannltstvqvfadseyqlpyvlgsahqgcfppfpadvfmipqygyltlnngsq |
| | | amgrssfycleyfpsqmlrtgnnftfsytfedvpfhssyahsqsldrlmnplidq |
| | | ylyylsktagglafsqagpttmrnqsrnwlpgpcfrqqrvserstennngdfswt |
| | | gttryhlngrdsamnpgpamashkddedrffpqngvlifgtpnatasnaplenvl |
| | | itdeeeirttnpvateeygivannkqdsstqattaivnaqgilpgmvwqdrdvyl |
| | | qgpiwakiphtdghfhpsplmggfglkhpppqilikntpvpsnppekftqeklna |
| | | fitqystgqvsveiewelqkenskrwnpevqytsnynksvnvdftvdtngmysep |
| | | rtigtryltrnl |
| 22 | Porcine4 AAV | atgtcgtttgttgatcaccctccagattggttggaagaggttggtgaaggccttc |
| | VP1 | acgagtttttggagctcgaagctggcccacccaaaccgaagcccaaccagcagaa |
| | nucleotide | gcaggacaacgcccgtggtcttgtactgcctggatataattatctgggacccttc |
| | sequence | aacggactcgacaagggagagcccgtcaaccgagcggacgctgttgcgcgagagc |
| | | acgacatctcgtacaacgagcagcttcaggcgggagacaacccctacctcaagta |
| | | caaccacgcggacgccgagtttcaggagaagctcaaggacgacacctcctttggg |
| | | ggcaacctcggaaaggcaatctttcaggccaagaaaagggttctcgaaccttttg |
| | | gcctggttgaagcgcctgttaagacggctccagccaagaagcggccgatagagaa |
| | | gtctccggcggaaccgagctcttcgaagggcatcggcaaggcgggccagcagcct |
| | | gcgagaaagcggctcaactttggtcagactggagacaccgactccgccgctgacc |
| | | cccagcctctcggagaaccacccgcagccccctctggtctgggaactggtacgat |
| | | ggcttcaggcggtggcgcaccaatggcagacaataacgaaggcgccgacggagtg |
| | | ggtaatgcctcgggaaattggcattgcgattccacatggctgggcgaccgagtca |
| | | tcaccaccagcacccgcacctgggccctgcccacctacaacaaccacctctacaa |
| | | gcaaatctcctctagcaattacggagccaacaacgacaaccactactttggctac |
| | | agcaccccctgggggtattttgacttcaaccgtttccactgccacttctctccgc |
| | | gagactggcagcggctcatcaacaacaactgggggttccggcccaagcgactcaa |
| | | cttcaagctgttcaacatccaagtcaaggaggtcacagaaacggacggcacgaag |
| | | acgatcgccaataaccttaccagcacggttcaagtctttgcggactcggagtacc |
| | | agctgccgtatgtcctcggctcggcacaccagggctgcttcccgccgttcccagc |
| | | ggacgtctttatgatcccacaatacggatacctgacgctgaacaacggcagccag |
| | | gcgatgggtcgctcttccttctactgcctggaatactttccgtcgcagatgctgc |
| | | ggacgggaaacaacttcacgttcagctacacctttgaggacgtgcccttccacag |
| | | cagctacgcgcacagccagagcctggaccggctgatgaacccgctcatcgaccag |
| | | tacctgtactacctgagcaagacagccggtggtctggcgttttctcaagcagggc |
| | | ccaccaccatgcggaaccagtccaggaactggctcccgggaccctgcttccgaca |
| | | gcagcgagtttcagaaagatccacagaaaacaacaacggagacttctcgtggaca |
| | | gggaccacgagataccatcttaacggtagagactcggcgatgaatccgggcccgg |
| | | ccatggccagtcacaaggacgacgaggacagattctttccgcaaaacggcgtgct |
| | | catctttggaacaccgaacgctactgcaagcaatgcacctctagagaatgtactg |
| | | atcaccgatgaggaggaaatcagaactacgaaccctgtagccacggaagagtacg |
| | | ggatcgttgctaacaacaaacaggacagcagtacacaggcgacgaccgcgattgt |
| | | caacgcacaaggcatactgcccggcatggtgtggcaggatcgagatgtgtacctg |
| | | caggggcccatctgggccaagatcccccacacggatggacacttccacccatcac |
| | | cgctcatgggcgggtttggccttaagcatccacctccgcagatcctcatcaagaa |
| | | cacgcctgtgccgtcgaaccctccagaaaagttcacccaggaaaaactaaatgcc |
| | | ttcatcacacagtactcgacgggccaggtcagcgtggagatcgagtgggagctgc |
| | | agaaggagaacagcaagcgctggaacccagaggtccagtacacgtccaactacaa |
| | | caagtccgtcaacgtggactttactgtggacaccaacggcatgtactcggagccc |
| | | cgcactatcggcacccgctacctcacccgcaacctgtaa |
| 23 | Porcine5 AAV | msfvdhppdwleeigeglkeflglepgppkpkpnqqkqdnarglvlpgynylgpg |
| | VP1 amino | ngldrgepvnradevarehdisyneqlqagdnpylkynhadaefqekladdtsfg |
| | acid | gnlgkavfqakkrvlepfglvedpvktaakgeriddhypkkkkarveeteagtsg |
| | sequence (GH | aqqlqipaqpasslgadtmsagggsplgdnnqgadgvgnasgdwhcdstwmgdrv |
| | loop region | itkstrtwvlpsynnhlykeihnngvdgstanayfgystpwgyfdfnrfhshwsp |
| | underlined) | rdwqrlinnywgfrprslkvkifniqvkevtvqdntttiannltstvqvftdndy |
| | | qlpyvigngtegclpafppqvftlpqygyatlnrnnsenpterssffcleyfpsk |
| | | mlrtgnnfeftysfeevpfhcsfapsqnlfklanplvdqylyrfvstdtsgavqf |
| | | kknlagryantyknwfpgpmcrtqgwytgtgiynnkgatsfntsnrmdlegasyq |
| | | vppqpngmtntvqdsnlyalentmifnaqnatpgtnttypeenllitsesetqpv |
| | | nrvayssggqiannnqntntaptagtynhqeilpgsvwmdrdvylqgpiwakipe |
| | | tgahfhpspamggfglkypppmmlikntpvpgnittfsdvpvqsfitqystgqvt |
| | | vemewelkkenskrwnpeiqytnnynnptfvdfapdtegeyrttraigtryltrp |
| | | l |
| 24 | Porcine5 AAV | atgtcgtttgttgatcaccctccagattggcttgaggagattggtgagggtctaa |
| | VP1 | aggagtttttgggactcgaacctggcccacccaaaccgaagcccaaccagcagaa |
| | nucleotide | gcaagacaacgcccgtggtcttgtactgcctggatataattacctgggacccggc |
| | sequence | aacggtctcgaccgcggagaacctgtcaaccgggccgacgaggtcgcgcgagagc |
| | | acgacatctcgtacaacgagcagctccaggcgggagacaacccctacctcaagta |
| | | caaccacgcggacgccgagtttcaagagaagctcgcggacgacacctccttcggg |
| | | ggcaacctcggcaaggcagtctttcaggccaagaaaagggttctcgaaccttttg |
| | | gcctggttgaggatcctgttaagacggctgctaaaggcgagcggatagacgacca |
| | | ctatoccaagaagaagaaggctcgggttgaagaaaccgaagctggaaccagcgga |
| | | gcccagcagctgcagatcccagcccaaccagcctcaagtttgggagctgatacaa |
| | | tgtctgcgggaggtggcagcccactgggcgacaataaccaaggcgccgatggagt |
| | | gggcaatgcctcgggagattggcattgcgattccacatggatgggggacagagtc |
| | | atcaccaagtccacccgaacctgggtgctgcccagctacaataaccacctgtaca |
| | | aagagatccacaacaacggggtcgacggcagcaccgccaacgcttactttggata |
| | | cagtaccccctgggggtactttgacttcaaccgcttccacagccactggagccct |
| | | cgagactggcagcgactcatcaacaactactggggcttcagaccccggtccctca |
| | | aggtcaagattttcaacatccaagtcaaagaggtcacggtgcaggacaacaccac |
| | | caccatcgccaacaacctcacctccaccgtccaagtgtttacggacaacgactac |
| | | cagctgccgtacgtcatcggcaacgggaccgaggggtgtctaccggccttccctc |
| | | cgcaggtctttacgctgccgcagtatggctacgcgacgctgaatcgcaacaatag |
| | | cgaaaatcccaccgagcgaagcagcttcttctgtctggagtactttcccagcaag |
| | | atgctgcggacgggcaacaactttgagttcacatacagcttcgaggaggtaccct |
| | | tccactgcagcttcgcgcccagccaaaacctcttcaaactggctaacccgttggt |
| | | ggatcagtacctttaccgcttcgtgagcacggacacctccggtgccgtccagttc |
| | | aaaaagaacctggcgggcagatacgccaacacctacaagaactggttoccaggac |
| | | ccatgtgccgaacccagggctggtacacaggaacgggtatatataacaacaaagg |
| | | cgctaccagctttaacacctcaaacagaatggacctagagggagccagttatcaa |
| | | gtgcctccccagcccaacgggatgacaaacacggttcaggacagcaacctttacg |
| | | cgctggaaaacaccatgatttttaacgcacaaaacgccaccccgggaacgaatac |
| | | aacgtatccggaggagaaccttttgataaccagtgagagcgagactcaacccgtg |
| | | aacagagtggcttacagctccggaggacaaatagccaacaacaatcagaatacca |
| | | acacggctcctactgcaggaacctacaaccaccaggaaatactacctggcagcgt |
| | | gtggatggacagggacgtgtacctccagggtcccatctgggccaagatcccagag |
| | | acaggggcacactttcatccttctccagccatgggcgggttcggactcaaatacc |
| | | cgcctcccatgatgctaatcaagaacacgccagtgcccggaaacatcaccacctt |
| | | ctcggacgtgcccgtccaaagttttattacccagtacagcaccggacaagtcacc |
| | | gtggagatggagtgggagctcaagaaggaaaactctaagaggtggaaccccgaga |
| | | tacagtacaccaacaactacaacaaccctacgttcgtggactttgctccagacac |
| | | agaaggagaatacaggaccactagggctattggaacccgctaccttacccgacct |
| | | ctgtaa |
| 25 | AAV9/AAV2 GH | maadgylpdwlednlsegirewwalkpgapqpkanqqhqdnarglvlpgykylgp |
| | loop VP1 | gngldkgepvnaadaaalehdkaydqqlkagdnpylkynhadaefqerlkedtsf |
| | amino acid | ggnlgravfqakkrlleplglveeaaktapgkkrpveqspqepdssagigksgaq |
| | sequence (GH | pakkrlnfgqtgdtesvpdpqpigeppaapsgvgsltmasgggapvadnnegadg |
| | loop region | vgsssgnwhcdsqwlgdrvittstrtwalptynnhlykqisnstsggssndnayf |
| | underlined) | gystpwgyfdfnrfhchfsprdwqrlinnnwgfrpkrlnfklfniqvkevtdnng |
| | | vktiannltstvqvftdsdyqlpyvlgsahegclppfpadvfmipqygyltlndg |
| | | sqavgrssfycleyfpsqmlrtgnnfqfsyefenvpfhssyahsqsldrlmnpli |
| | | dqylyylsrtntpsgtttqsrlqfsqagasdirdqsrnwlpgpcyrqqrvsktsa |
| | | dnnnseyswtgatkyhlngrdslvnpgpamashkddeekffpqsgvlifgkqgse |
| | | ktnvdiekvmitdeeeirttnpvateqygsvstnlqrgnrqaatadvntqgvlpg |
| | | mvwqdrdvylqgpiwakiphtdghfhpsplmggfgmkhpppqilikntpvpadpp |
| | | tafnkdklnsfitqystgqvsveiewelqkenskrwnpeiqytsnyyksnnvefa |
| | | vntegvyseprpigtryltrnl |
| 26 | AAV9/AAV2 GH | atggctgccgatggttatcttccagattggctcgaggacaaccttagtgaaggaa |
| | loop VP1 | ttcgcgagtggtgggctttgaaacctggagcccctcaacccaaggcaaatcaaca |
| | nucleotide | acatcaagacaacgctcgaggtcttgtgcttccgggttacaaataccttggaccc |
| | sequence | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgttccttt |
| | | ccacagcagctacgctcacagccagagtctggaccgtctcatgaatcctctcatc |
| | | gaccagtacctgtattacttgagcagaacaaacactccaagtggaaccaccacgc |
| | | agtcaaggcttcagttttctcaggccggagcgagtgacattcgggaccagtctag |
| | | gaactggcttcctggaccctgttaccgccagcagcgagtatcaaagacatctgcg |
| | | gataacaacaacagtgaatactcgtggactggagctaccaagtaccacctcaatg |
| | | gcagagactctctggtgaatccgggcccggccatggcaagccacaaggacgatga |
| | | agaaaagttttttcctcagagcggggttctcatctttgggaagcaaggctcagag |
| | | aaaacaaatgtggacattgaaaaggtcatgattacagacgaagaggaaatcagga |
| | | caaccaatcccgtggctacggagcagtatggttctgtatctaccaacctccagag |
| | | aggcaacagacaagcagctaccgcagatgtcaacacacaaggcgttcttccaggc |
| | | atggtctggcaggacagagatgtgtaccttcaggggcccatctgggcaaagattc |
| | | cacacacggacggacattttcacccctctcccctcatgggtggattcggaatgaa |
| | | gcacccgcctcctcagatcctcatcaaaaacacacctgtacctgcggatcctcca |
| | | acggccttcaacaaggacaagctgaactctttcatcacccagtattctactggcc |
| | | aagtcagcgtggagatcgagtgggagctgcagaaggaaaacagcaagcgctggaa |
| | | cccggagatccagtacacttccaactattacaagtctaataatgttgaatttgct |
| | | gttaatactgaaggtgtatatagtgaaccccgccccattggcaccagatacctga |
| | | ctcgtaatctgtaa |
| 27 | AAV9/AAV3B | maadgylpdwlednlsegirewwalkpgapqpkanqqhqdnarglvlpgykylgp |
| | GH loop VP1 | gngldkgepvnaadaaalehdkaydqqlkagdnpylkynhadaefqerlkedtsf |
| | amino acid | ggnlgravfqakkrlleplglveeaaktapgkkrpveqspqepdssagigksgaq |
| | sequence (GH | pakkrlnfgqtgdtesvpdpqpigeppaapsgvgsltmasgggapvadnnegadg |
| | loop region | vgsssgnwhcdsqwlgdrvittstrtwalptynnhlykqisnstsggssndnayf |
| | underlined) | gystpwgyfdfnrfhchfsprdwqrlinnnwgfrpkrlnfklfniqvkevtdnng |
| | | vktiannltstvqvftdsdyqlpyvlgsahegclppfpadvfmipqygyltlndg |
| | | sqavgrssfycleyfpsqmlrtgnnfqfsyefenvpfhssyahsqsldrlmnpli |
| | | dqylyylnrtqgttsgttnqsrllfsqagpqsmslqarnwlpgpcyrqqrlskta |
| | | ndnnnsnfpwtaaskyhlngrdslvnpgpamashkddeekffpmhgnlifgkegt |
| | | tasnaeldnvmitdeeeirttnpvateqygtvannlqssntapttrtvndqgalp |
| | | gmvwqdrdvylqgpiwakiphtdghfhpsplmggfglkhpppqilikntpvpadp |
| | | ptafnkdklnsfitqystgqvsveiewelqkenskrwnpeiqytsnyyksnnvef |
| | | avntegvyseprpigtryltrnl |
| 28 | AAV9/AAV3B | atggctgccgatggttatcttccagattggctcgaggacaaccttagtgaaggaa |
| | GH loop VP1 | ttcgcgagtggtgggctttgaaacctggagcccctcaacccaaggcaaatcaaca |
| | nucleotide | acatcaagacaacgctcgaggtcttgtgcttccgggttacaaataccttggaccc |
| | sequence | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtaccttt |
| | | tcacagcagctacgctcacagccagagtttggatcgcttgatgaatcctcttatt |
| | | gatcagtatctgtactacctgaacagaacgcaaggaacaacctctggaacaacca |
| | | accaatcacggctgctttttagccaggctgggcctcagtctatgtctttgcaggc |
| | | cagaaattggctacctgggccctgctaccggcaacagagactttcaaagactgct |
| | | aacgacaacaacaacagtaactttccttggacagcggccagcaaatatcatctca |
| | | atggccgcgactcgctggtgaatccaggaccagctatggccagtcacaaggacga |
| | | tgaagaaaaatttttccctatgcacggcaatctaatatttggcaaagaagggaca |
| | | acggcaagtaacgcagaattagataatgtaatgattacggatgaagaagagattc |
| | | gtaccaccaatcctgtggcaacagagcagtatggaactgtggcaaataacttgca |
| | | gagctcaaatacagctcccacgactagaactgtcaatgatcagggggccttacct |
| | | ggcatggtgtggcaagatcgtgacgtgtaccttcaaggacctatctgggcaaaga |
| | | ttcctcacacggatggacactttcatccttctcctctgatgggaggctttggact |
| | | gaaacatccgcctcctcaaatcctcatcaaaaacacacctgtacctgcggatcct |
| | | ccaacggccttcaacaaggacaagctgaactctttcatcacccagtattctactg |
| | | gccaagtcagcgtggagatcgagtgggagctgcagaaggaaaacagcaagcgctg |
| | | gaacccggagatccagtacacttccaactattacaagtctaataatgttgaattt |
| | | gctgttaatactgaaggtgtatatagtgaaccccgccccattggcaccagatacc |
| | | tgactcgtaatctgtaa |
| 29 | AAV9/AAV4 GH | maadgylpdwlednlsegirewwalkpgapqpkanqqhqdnarglvlpgykylgp |
| | loop VP1 | gngldkgepvnaadaaalehdkaydqqlkagdnpylkynhadaefqerlkedtsf |
| | amino acid | ggnlgravfqakkrlleplglveeaaktapgkkrpveqspqepdssagigksgaq |
| | sequence (GH | pakkrlnfgqtgdtesvpdpqpigeppaapsgvgsltmasgggapvadnnegadg |
| | loop region | vgsssgnwhcdsqwlgdrvittstrtwalptynnhlykqisnstsggssndnayf |
| | underlined) | gystpwgyfdfnrfhchfsprdwqrlinnnwgfrpkrlnfklfniqvkevtdnng |
| | | vktiannltstvqvftdsdyqlpyvlgsahegclppfpadvfmipqygyltlndg |
| | | sqavgrssfycleyfpsqmlrtgnnfqfsyefenvpfhsmyahsqsldrlmnpli |
| | | dqylwglqstttgttlnagtattnftklrptnfsnfkknwlpgpsikqqgfskta |
| | | nqnykipatgsdslikyethstldgrwsaltpgppmatagpadskfsnsqlifag |
| | | pkqngntatvpgtliftseeelaatnatdtdmwgnlpggdqsnsnlptvdrltal |
| | | gavpgmvwqnrdiyyqgpiwakiphtdghfhpspliggfgmkhpppqilikntpv |
| | | padpptafnkdklnsfitqystgqvsveiewelqkenskrwnpeiqytsnyyksn |
| | | nvefavntegvyseprpigtryltrnl |
| 30 | AAV9/AAV4 GH | atggctgccgatggttatcttccagattggctcgaggacaaccttagtgaaggaa |
| | loop VP1 | ttcgcgagtggtgggctttgaaacctggagcccctcaacccaaggcaaatcaaca |
| | nucleotide | acatcaagacaacgctcgaggtcttgtgcttccgggttacaaataccttggaccc |
| | sequence | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacgagtttgagaacgtgccttt |
| | | ccactcgatgtacgcgcacagccagagcctggaccggctgatgaaccctctcatc |
| | | gaccagtacctgtggggactgcaatcgaccaccaccggaaccaccctgaatgccg |
| | | ggactgccaccaccaactttaccaagctgcggcctaccaacttttccaactttaa |
| | | aaagaactggctgcccgggccttcaatcaagcagcagggcttctcaaagactgcc |
| | | aatcaaaactacaagatccctgccaccgggtcagacagtctcatcaaatacgaga |
| | | cgcacagcactctggacggaagatggagtgccctgacccccggacctccaatggc |
| | | cacggctggacctgcggacagcaagttcagcaacagccagctcatctttgcgggg |
| | | cctaaacagaacggcaacacggccaccgtacccgggactctgatcttcacctctg |
| | | aggaggagctggcagccaccaacgccaccgatacggacatgtggggcaacctacc |
| | | tggcggtgaccagagcaacagcaacctgccgaccgtggacagactgacagccttg |
| | | ggagccgtgcctggaatggtctggcaaaacagagacatttactaccagggtccca |
| | | tttgggccaagattcctcataccgatggacactttcacccctcaccgctgattgg |
| | | tgggtttgggatgaagcacccgcctcctcagatcctcatcaaaaacacacctgta |
| | | cctgcggatcctccaacggccttcaacaaggacaagctgaactctttcatcaccc |
| | | agtattctactggccaagtcagcgtggagatcgagtgggagctgcagaaggaaaa |
| | | cagcaagcgctggaacccggagatccagtacacttccaactattacaagtctaat |
| | | aatgttgaatttgctgttaatactgaaggtgtatatagtgaaccccgccccattg |
| | | gcaccagatacctgactcgtaatctgtaa |
| 31 | AAV9/AAV5 GH | maadgylpdwlednlsegirewwalkpgapqpkanqqhqdnarglvlpgykylgp |
| | loop VP1 | gngldkgepvnaadaaalehdkaydqqlkagdnpylkynhadaefqerlkedtsf |
| | amino acid | ggnlgravfqakkrlleplglveeaaktapgkkrpveqspqepdssagigksgaq |
| | sequence (GH | pakkrlnfgqtgdtesvpdpqpigeppaapsgvgsltmasgggapvadnnegadg |
| | loop region | vgsssgnwhcdsqwlgdrvittstrtwalptynnhlykqisnstsggssndnayf |
| | underlined) | gystpwgyfdfnrfhchfsprdwqrlinnnwgfrpkrlnfklfniqvkevtdnng |
| | | vktiannltstvqvftdsdyqlpyvlgsahegclppfpadvfmipqygyltlndg |
| | | sqavgrssfycleyfpsqmlrtgnnfqfsyefenvpfhssfapsqnlfklanplv |
| | | dqylyrfvstnntggvqfnknlagryantyknwfpgpmgrtqgwnlgsgvnrasv |
| | | safattnrmelegasyqvppqpngmtnnlqgsntyalentmifnsqpanpgttat |
| | | ylegnmlitsesetqpvnrvaynvggqmatnnqssttapatgtynlqeivpgsvw |
| | | merdvylqgpiwakipetgahfhpspamggfgmkhpppqilikntpvpadpptaf |
| | | nkdklnsfitqystgqvsveiewelqkenskrwnpeiqytsnyyksnnvefavnt |
| | | egvyseprpigtryltrnl |
| 32 | AAV9/AAV5 GH | atgtcttttgttgatcaccctccagattggttggaagaagttggtgaaggtcttc |
| | loop VP1 | gcgagtttttgggccttgaagcgggcccaccgaaaccaaaacccaatcagcagca |
| | nucleotide | tcaagatcaagcccgtggtcttgtgctgcctggttataactatctcggacccgga |
| | sequence | aacggtctcgatcgaggagagcctgtcaacagggcagacgaggtcgcgcgagagc |
| | | acgacatctcgtacaacgagcagcttgaggcgggagacaacccctacctcaagta |
| | | caaccacgcggacgccgagtttcaggagaagctcgccgacgacacatccttcggg |
| | | ggaaacctcggaaaggcagtctttcaggccaagaaaagggttctcgaaccttttg |
| | | gcctggttgaagagggtgctaagacggcccctaccggaaagcggatagacgacca |
| | | ctttccaaaaagaaagaaggctcggaccgaagaggactccaagccttccacctcg |
| | | tcagacgccgaagctggacccagcggatcccagcagctgcaaatcccagcccaac |
| | | cagcctcaagtttgggagctgatacaatgtctgcgggaggtggcggcccattggg |
| | | cgacaataaccaaggtgccgatggagtgggcaatgcctcgggagattggcattgc |
| | | gattccacgtggatgggggacagagtcgtcaccaagtccacccgaacctgggtgc |
| | | tgcccagctacaacaaccaccagtaccgagagatcaaaagcggctccgtcgacgg |
| | | aagcaacgccaacgcctactttggatacagcaccccctgggggtactttgacttt |
| | | aaccgcttccacagccactggagcccccgagactggcaaagactcatcaacaact |
| | | actggggcttcagaccccggtccctcagagtcaaaatcttcaacattcaagtcaa |
| | | agaggtcacggtgcaggactccaccaccaccatcgccaacaacctcacctccacc |
| | | gtccaagtgtttacggacgacgactaccagctgccctacgtcgtcggcaacggga |
| | | ccgagggatgcctgccggccttccctccgcaggtctttacgctgccgcagtacgg |
| | | ttacgcgacgctgaaccgcgacaacacagaaaatcccaccgagaggagcagcttc |
| | | ttctgcctagagtactttcccagcaagatgctgagaacgggcaacaactttgagt |
| | | ttacctacaactttgaggaggtgcccttccactccagcttcgctcccagtcagaa |
| | | cctgttcaagctggccaacccgctgatcgaccagtacctgtactacctgagcaag |
| | | acagccggtggtctggcgttttctcaagcagggcccaccaccatgcggaaccagt |
| | | ccaggaactggctcccgggaccctgcttccgacagcagcgagtttcagaaagatc |
| | | cacagaaaacaacaacggagacttctcgtggacagggaccacgagataccatctt |
| | | aacggtagagactcggcgatgaatccgggcccggccatggccagtcacaaggacg |
| | | acgaggacagattctttccgcaaaacggcgtgctcatctttggaacaccgaacgc |
| | | tactgcaagcaatgcacctctagagaatgtactgatcaccgatgaggaggaaatc |
| | | agaactacgaaccctgtagccacggaagagtacgggatcgttgctaacaacaaac |
| | | aggacagcagtacacaggcgacgaccgcgattgtcaacgcacaaggcatactgcc |
| | | cggcatggtgtggcaggatcgagatgtgtacctgcaggggcccatctgggccaag |
| | | atcccccacacggatggacacttccacccatcaccgctcatgggcgggtttggcc |
| | | ttaagcatccacctccgcagatcctcatcaagaacacgcctgtgccgggaaatat |
| | | caccagcttctcggacgtgcccgtcagcagcttcatcacccagtacagcaccggg |
| | | caggtcaccgtggagatggagtgggagctcaagaaggaaaactccaagaggtgga |
| | | acccagagatccagtacacaaacaactacaacgacccccagtttgtggactttgc |
| | | cccggacagcaccggggaatacagaaccaccagacctatcggaacccgatacctt |
| | | acccgacccctttaa |
| 33 | AAV9/AAV6 GH | maadgylpdwlednlsegirewwalkpgapqpkanqqhqdnarglvlpgykylgp |
| | loop VP1 | gngldkgepvnaadaaalehdkaydqqlkagdnpylkynhadaefqerlkedtsf |
| | amino acid | ggnlgravfqakkrlleplglveeaaktapgkkrpveqspqepdssagigksgaq |
| | sequence (GH | pakkrlnfgqtgdtesvpdpqpigeppaapsgvgsltmasgggapvadnnegadg |
| | loop region | vgsssgnwhcdsqwlgdrvittstrtwalptynnhlykqisnstsggssndnayf |
| | underlined) | gystpwgyfdfnrfhchfsprdwqrlinnnwgfrpkrlnfklfniqvkevtdnng |
| | | vktiannltstvqvftdsdyqlpyvlgsahegclppfpadvfmipqygyltlndg |
| | | sqavgrssfycleyfpsqmlrtgnnfqfsytfedvpfhssyahsqsldrlmnpli |
| | | dqylyylnrtqnqsgsaqnkdllfsrgspagmsvqpknwlpgpcyrqqrvsktkt |
| | | dnnnsnftwtgaskynlngresiinpgtamashkddkdkffpmsgvmifgkesag |
| | | asntaldnvmitdeeeikatnpvaterfgtvavnlqssstdpatgdvhvmgalpg |
| | | mvwqdrdvylqgpiwakiphtdghfhpsplmggfglkhpppqilikntpvpadpp |
| | | tafnkdklnsfitqystgqvsveiewelqkenskrwnpeiqytsnyyksnnvefa |
| | | vntegvyseprpigtryltrnl |
| 34 | AAV9/AAV6 GH | atggctgccgatggttatcttccagattggctcgaggacaaccttagtgaaggaa |
| | loop VP1 | ttcgcgagtggtgggctttgaaacctggagcccctcaacccaaggcaaatcaaca |
| | nucleotide | acatcaagacaacgctcgaggtcttgtgcttccgggttacaaataccttggaccc |
| | sequence | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacaccttcgaggacgtgccttt |
| | | ccacagcagctacgcgcacagccagagcctggaccggctgatgaatcctctcatc |
| | | gaccagtacctgtattacctgaacagaactcagaatcagtccggaagtgcccaaa |
| | | acaaggacttgctgtttagccgggggtctccagctggcatgtctgttcagcccaa |
| | | aaactggctacctggaccctgttaccggcagcagcgcgtttctaaaacaaaaaca |
| | | gacaacaacaacagcaactttacctggactggtgcttcaaaatataaccttaatg |
| | | ggcgtgaatctataatcaaccctggcactgctatggcctcacacaaagacgacaa |
| | | agacaagttctttcccatgagcggtgtcatgatttttggaaaggagagcgccgga |
| | | gcttcaaacactgcattggacaatgtcatgatcacagacgaagaggaaatcaaag |
| | | ccactaaccccgtggccaccgaaagatttgggactgtggcagtcaatctccagag |
| | | cagcagcacagaccctgcgaccggagatgtgcatgttatgggagccttacctgga |
| | | atggtgtggcaagacagagacgtatacctgcagggtcctatttgggccaaaattc |
| | | ctcacacggatggacactttcacccgtctcctctcatgggcggctttggacttaa |
| | | gcacccgcctcctcagatcctcatcaaaaacacgcctgttcctgoggatcctcca |
| | | acggccttcaacaaggacaagctgaactctttcatcacccagtattctactggcc |
| | | aagtcagcgtggagatcgagtgggagctgcagaaggaaaacagcaagcgctggaa |
| | | cccggagatccagtacacttccaactattacaagtctaataatgttgaatttgct |
| | | gttaatactgaaggtgtatatagtgaaccccgccccattggcaccagatacctga |
| | | ctcgtaatctgtaa |
| 35 | AAV9/AAV7 GH | maadgylpdwlednlsegirewwalkpgapqpkanqqhqdnarglvlpgykylgp |
| | loop VP1 | gngldkgepvnaadaaalehdkaydqqlkagdnpylkynhadaefqerlkedtsf |
| | amino acid | ggnlgravfqakkrlleplglveeaaktapgkkrpveqspqepdssagigksgaq |
| | sequence (GH | pakkrlnfgqtgdtesvpdpqpigeppaapsgvgsltmasgggapvadnnegadg |
| | loop region | vgsssgnwhcdsqwlgdrvittstrtwalptynnhlykqisnstsggssndnayf |
| | underlined) | gystpwgyfdfnrfhchfsprdwqrlinnnwgfrpkrlnfklfniqvkevtdnng |
| | | vktiannltstvqvftdsdyqlpyvlgsahegclppfpadvfmipqygyltlndg |
| | | sqavgrssfycleyfpsqmlrtgnnfqfsysfedvpfhssyahsqsldrlmnpli |
| | | dqylyylartqsnpggtagnrelqfyqggpstmaeqaknwlpgpcfrqqrvsktl |
| | | dannnsnfawtgatkyhlngrnslvnpgvamathkddedrffpssgvlifgktga |
| | | tnkttlenvlmtneeeirptnpvateeygivssnlqaantaaqtqvvnnqgalpg |
| | | mvwqnrdvylqgpiwakiphtdgnfhpsplmggfglkhpppqilikntpvpadpp |
| | | tafnkdklnsfitqystgqvsveiewelqkenskrwnpeiqytsnyyksnnvefa |
| | | vntegvyseprpigtryltrnl |
| 36 | AAV9/AAV7 GH | atggctgccgatggttatcttccagattggctcgaggacaaccttagtgaaggaa |
| | loop VP1 | ttcgcgagtggtgggctttgaaacctggagcccctcaacccaaggcaaatcaaca |
| | nucleotide | acatcaagacaacgctcgaggtcttgtgcttccgggttacaaataccttggaccc |
| | sequence | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctcttac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagttcagctacagcttcgaggacgtgccttt |
| | | ccacagcagctacgcacacagccagagcctggaccggctgatgaatcccctcatc |
| | | gaccagtacttgtactacctggccagaacacagagtaacccaggaggcacagctg |
| | | gcaatcgggaactgcagttttaccagggcgggccttcaactatggccgaacaagc |
| | | caagaattggttacctggaccttgcttccggcaacaaagagtctccaaaacgctg |
| | | gatcaaaacaacaacagcaactttgcttggactggtgccaccaaatatcacctga |
| | | acggcagaaactcgttggttaatcccggcgtcgccatggcaactcacaaggacga |
| | | cgaggaccgctttttcccatccagcggagtcctgatttttggaaaaactggagca |
| | | actaacaaaactacattggaaaatgtgttaatgacaaatgaagaagaaattcgtc |
| | | ctactaatcctgtagccacggaagaatacgggatagtcagcagcaacttacaagc |
| | | ggctaatactgcagcccagacacaagttgtcaacaaccagggagccttacctggc |
| | | atggtctggcagaaccgggacgtgtacctgcagggtcccatctgggccaagattc |
| | | ctcacacggatggcaactttcacccgtctcctttgatgggcggctttggacttaa |
| | | acatccgcctcctcagatcctgatcaagaacactcccgttcccgcggatcctcca |
| | | acggccttcaacaaggacaagctgaactctttcatcacccagtattctactggcc |
| | | aagtcagcgtggagatcgagtgggagctgcagaaggaaaacagcaagcgctggaa |
| | | cccggagatccagtacacttccaactattacaagtctaataatgttgaatttgct |
| | | gttaatactgaaggtgtatatagtgaaccccgccccattggcaccagatacctga |
| | | ctcgtaatctgtaa |
| 37 | AAV9/AAV8 GH | maadgylpdwlednlsegirewwalkpgapqpkanqqhqdnarglvlpgykylgp |
| | loop VP1 | gngldkgepvnaadaaalehdkaydqqlkagdnpylkynhadaefqerlkedtsf |
| | amino acid | ggnlgravfqakkrlleplglveeaaktapgkkrpveqspqepdssagigksgaq |
| | sequence (GH | pakkrlnfgqtgdtesvpdpqpigeppaapsgvgsltmasgggapvadnnegadg |
| | loop region | vgsssgnwhcdsqwlgdrvittstrtwalptynnhlykqisnstsggssndnayf |
| | underlined) | gystpwgyfdfnrfhchfsprdwqrlinnnwgfrpkrlnfklfniqvkevtdnng |
| | | vktiannltstvqvftdsdyqlpyvlgsahegclppfpadvfmipqygyltlndg |
| | | sqavgrssfycleyfpsqmlrtgnnfqftytfedvpfhssyahsqsldrlmnpli |
| | | dqylyylsrtqttggtantqtlgfsqggpntmangaknwlpgpcyrqqrvstttg |
| | | qnnnsnfawtagtkyhlngrnslanpgiamathkddeerffpsngilifgkqnaa |
| | | rdnadysdvmltseeeikttnpvateeygivadnlqqqntapqigtvnsqgalpg |
| | | mvwqnrdvylqgpiwakiphtdgnfhpsplmggfglkhpppqilikntpvpadpp |
| | | tafnkdklnsfitqystgqvsveiewelqkenskrwnpeiqytsnyyksnnvefa |
| | | vntegvyseprpigtryltrnl |
| 38 | AAV9/AAV8 GH | atggctgccgatggttatcttccagattggctcgaggacaaccttagtgaaggaa |
| | loop VP1 | ttcgcgagtggtgggctttgaaacctggagcccctcaacccaaggcaaatcaaca |
| | nucleotide | acatcaagacaacgctcgaggtcttgtgcttccgggttacaaataccttggaccc |
| | sequence | ggcaacggactcgacaagggggagccggtcaacgcagcagacgcggcggccctcg |
| | | agcacgacaaggcctacgaccagcagctcaaggccggagacaacccgtacctcaa |
| | | gtacaaccacgccgacgccgagttccaggagcggctcaaagaagatacgtctttt |
| | | gggggcaacctcgggcgagcagtcttccaggccaaaaagaggcttcttgaacctc |
| | | ttggtctggttgaggaagcggctaagacggctcctggaaagaagaggcctgtaga |
| | | gcagtctcctcaggaaccggactcctccgcgggtattggcaaatcgggtgcacag |
| | | cccgctaaaaagagactcaatttcggtcagactggcgacacagagtcagtcccag |
| | | accctcaaccaatcggagaacctcccgcagccccctcaggtgtgggatctottac |
| | | aatggcttcaggtggtggcgcaccagtggcagacaataacgaaggtgccgatgga |
| | | gtgggtagttcctcgggaaattggcattgcgattcccaatggctgggggacagag |
| | | tcatcaccaccagcacccgaacctgggccctgcccacctacaacaatcacctcta |
| | | caagcaaatctccaacagcacatctggaggatcttcaaatgacaacgcctacttc |
| | | ggctacagcaccccctgggggtattttgacttcaacagattccactgccacttct |
| | | caccacgtgactggcagcgactcatcaacaacaactggggattccggcctaagcg |
| | | actcaacttcaagctcttcaacattcaggtcaaagaggttacggacaacaatgga |
| | | gtcaagaccatcgccaataaccttaccagcacggtccaggtcttcacggactcag |
| | | actatcagctcccgtacgtgctcgggtcggctcacgagggctgcctcccgccgtt |
| | | cccagcggacgttttcatgattcctcagtacgggtatctgacgcttaatgatgga |
| | | agccaggccgtgggtcgttcgtccttttactgcctggaatatttcccgtcgcaaa |
| | | tgctaagaacgggtaacaacttccagtttacttacaccttcgaggacgtgccttt |
| | | ccacagcagctacgcccacagccagagcttggaccggctgatgaatcctctgatt |
| | | gaccagtacctgtactacttgtctcggactcaaacaacaggaggcacggcaaata |
| | | cgcagactctgggcttcagccaaggtgggcctaatacaatggccaatcaggcaaa |
| | | gaactggctgccaggaccctgttaccgccaacaacgcgtctcaacgacaaccggg |
| | | caaaacaacaatagcaactttgcctggactgctgggaccaaataccatctgaatg |
| | | gaagaaattcattggctaatcctggcatcgctatggcaacacacaaagacgacga |
| | | ggagcgtttttttcccagtaacgggatcctgatttttggcaaacaaaatgctgcc |
| | | agagacaatgcggattacagcgatgtcatgctcaccagcgaggaagaaatcaaaa |
| | | ccactaaccctgtggctacagaggaatacggtatcgtggcagataacttgcagca |
| | | gcaaaacacggctcctcaaattggaactgtcaacagccagggggccttacccggt |
| | | atggtctggcagaaccgggacgtgtacctgcagggtcccatctgggccaagattc |
| | | ctcacacggacggcaacttccacccgtctccgctgatgggcggctttggcctgaa |
| | | acatcctccgcctcagatcctgatcaagaacacgcctgtacctgcggatcctcca |
| | | acggccttcaacaaggacaagctgaactctttcatcacccagtattctactggcc |
| | | aagtcagcgtggagatcgagtgggagctgcagaaggaaaacagcaagcgctggaa |
| | | cccggagatccagtacacttccaactattacaagtctaataatgttgaatttgct |
| | | gttaatactgaaggtgtatatagtgaaccccgccccattggcaccagatacctga |
| | | ctcgtaatctgtaa |
| 39 | AAVrh10/AAV4 | maadgylpdwlednlsegirewwdlkpgapkpkanqqkqddgrglvlpgykylgp |
| | GH loop VP1 | fngldkgepvnaadaaalehdkaydqqlkagdnpylrynhadaefqerlqedtsf |
| | amino acid | ggnlgravfqakkrvleplglveegaktapgkkrpvepspqrspdsstgigkkgq |
| | sequence (GH | qpakkrlnfgqtgdsesvpdpqpigeppagpsglgsgtmaagggapmadnnegad |
| | loop region | gvgsssgnwhcdstwlgdrvittstrtwalptynnhlykqisngtsggstndnty |
| | underlined) | fgystpwgyfdfnrfhchfsprdwqrlinnnwgfrpkrlnfklfniqvkevtqne |
| | | gtktiannltstiqvftdseyqlpyvlgsahqgclppfpadvfmipqygyltlnn |
| | | gsqavgrssfycleyfpsqmlrtgnnfefsyqfedvpfhsmyahsqsldrlmnpl |
| | | idqylwglqstttgttlnagtattnftklrptnfsnfkknwlpgpsikqqgfskt |
| | | anqnykipatgsdslikyethstldgrwsaltpgppmatagpadskfsnsqlifa |
| | | gpkqngntatvpgtliftseeelaatnatdtdmwgnlpggdqsnsnlptvdrlta |
| | | lgavpgmvwqnrdiyyqgpiwakiphtdghfhpspliggfglkhpppqilikntp |
| | | vpadppttfsqaklasfitqystgqvsveiewelqkenskrwnpeiqytsnyyks |
| | | tnvdfavntdgtyseprpigtryltrnl |
| 40 | AAVrh10/AAV4 | atggctgccgatggttatcttccagattggctcgaggacaacctctctgagggca |
| | GH loop VP1 | ttcgcgagtggtgggacttgaaacctggagccccgaaacccaaagccaaccagca |
| | nucleotide | aaagcaggacgacggccggggtctggtgcttcctggctacaagtacctcggaccc |
| | sequence | ttcaacggactcgacaagggggagcccgtcaacgcggcggacgcagcggcccttg |
| | | agcacgacaaggcctacgaccagcagctcaaagcgggtgacaatccgtacctgcg |
| | | gtataaccacgccgacgccgagtttcaggagcgtctgcaagaagatacgtctttt |
| | | gggggcaacctcggacgagcagtcttccaggccaagaagcgggttctcgaacctc |
| | | tcggtctggttgaggaaggcgctaagacggctcctggaaagaagagaccggtaga |
| | | gccatcaccccagcgttctccagactcctctacgggcatcggcaagaaaggccag |
| | | cagcccgcgaaaaagagactcaactttgggcagactggcgactcagagtcagtgc |
| | | ccgaccctcaaccaatcggagaaccccccgcaggcccctctggtctgggatctgg |
| | | tacaatggcagcaggcggtggcgctcccatggcagacaataacgaaggcgccgac |
| | | ggagtgggtagttcctcaggaaattggcattgcgattccacatggctgggcgaca |
| | | gagtcatcaccaccagcacccgaacctgggccctccccacctacaacaaccacct |
| | | ctacaagcaaatctccaacgggacttcgggaggaagcaccaacgacaacacctac |
| | | ttcggctacagcaccccctgggggtattttgactttaacagattccactgccact |
| | | tctcaccacgtgactggcagcgactcatcaacaacaactggggattccggcccaa |
| | | gagactcaacttcaagctcttcaacatccaggtcaaggaggtcacgcagaatgaa |
| | | ggcaccaagaccatcgccaataaccttaccagcacgattcaggtctttacggact |
| | | cggaataccagctcccgtacgtcctcggctctgcgcaccagggctgcctgcctcc |
| | | gttcccggcggacgtcttcatgattcctcagtacgggtacctgactctgaacaat |
| | | ggcagtcaggccgtgggccgttcctccttctactgcctggagtactttccttctc |
| | | aaatgctgagaacgggcaacaactttgagttcagctaccagtttgaggacgtgcc |
| | | tttccactcgatgtacgcgcacagccagagcctggaccggctgatgaaccctctc |
| | | atcgaccagtacctgtggggactgcaatcgaccaccaccggaaccaccctgaatg |
| | | ccgggactgccaccaccaactttaccaagctgcggcctaccaacttttccaactt |
| | | taaaaagaactggctgcctgggccttcaatcaagcagcagggcttctcaaagact |
| | | gccaatcaaaactacaagatccctgccaccgggtcagacagtctcatcaaatacg |
| | | agacgcacagcactctggacggaagatggagtgccctgacccccggacctccaat |
| | | ggccacggctggacctgcggacagcaagttcagcaacagccagctcatctttgcg |
| | | gggcctaaacagaacggcaacacggccaccgtacccgggactctgatcttcacct |
| | | ctgaggaggagctggcagccaccaacgccaccgatacggacatgtggggcaacct |
| | | acctggcggtgaccagagcaacagcaacctgccgaccgtggacagactgacagcc |
| | | ttgggagccgtgcctggaatggtctggcaaaacagagacatttactaccagggtc |
| | | ccatttgggccaagattcctcataccgatggacactttcacccctcaccgctgat |
| | | tggtgggtttgggctgaaacacccgcctcctcagatcctgattaagaatacacct |
| | | gttcccgcggatcctccaactaccttcagtcaagctaagctggcgtcgttcatca |
| | | cgcagtacagcaccggacaggtcagcgtggaaattgaatgggagctgcagaaaga |
| | | aaacagcaaacgctggaacccagagattcaatacacttccaactactacaaatct |
| | | acaaatgtggactttgctgttaacacagatggcacttattctgagcctcgcccca |
| | | tcggcacccgttacctcacccgtaatctgtaa |
| 41 | AAVrh10/AAV9 | maadgylpdwlednlsegirewwdlkpgapkpkanqqkqddgrglvlpgykylgp |
| | GH loop VP1 | fngldkgepvnaadaaalehdkaydqqlkagdnpylrynhadaefqerlqedtsf |
| | amino acid | ggnlgravfqakkrvleplglveegaktapgkkrpvepspqrspdsstgigkkgq |
| | sequence (GH | qpakkrlnfgqtgdsesvpdpqpigeppagpsglgsgtmaagggapmadnnegad |
| | loop region | gvgsssgnwhcdstwlgdrvittstrtwalptynnhlykqisngtsggstndnty |
| | underlined) | fgystpwgyfdfnrfhchfsprdwqrlinnnwgfrpkrlnfklfniqvkevtqne |
| | | gtktiannltstiqvftdseyqlpyvlgsahqgclppfpadvfmipqygyltlnn |
| | | gsqavgrssfycleyfpsqmlrtgnnfefsyqfedvpfhssyahsqsldrlmnpl |
| | | idqylyylsktingsgqnqqtlkfsvagpsnmavqgrnyipgpsyrqqrvsttvt |
| | | qnnnsefawpgasswalngrnslmnpgpamashkegedrffplsgslifgkqgtg |
| | | rdnvdadkvmitneeeikttnpvatesygqvatnhqsaqaqaqtgwvqnqgilpg |
| | | mvwqdrdvylqgpiwakiphtdgnfhpsplmggfgmkhpppqilikntpvpadpp |
| | | ttfsqaklasfitqystgqvsveiewelqkenskrwnpeiqytsnyykstnvdfa |
| | | vntdgtyseprpigtryltrnl |
| 42 | AAVrh10/AAV9 | atggctgccgatggttatcttccagattggctcgaggacaacctctctgagggca |
| | GH loop VP1 | ttcgcgagtggtgggacttgaaacctggagccccgaaacccaaagccaaccagca |
| | nucleotide | aaagcaggacgacggccggggtctggtgcttcctggctacaagtacctcggaccc |
| | sequence | ttcaacggactcgacaagggggagcccgtcaacgcggcggacgcagcggccctcg |
| | | agcacgacaaggcctacgaccagcagctcaaagcgggtgacaatccgtacctgcg |
| | | gtataaccacgccgacgccgagtttcaggagcgtctgcaagaagatacgtctttt |
| | | gggggcaacctcgggcgagcagtcttccaggccaagaagcgggttctcgaacctc |
| | | tcggtctggttgaggaaggcgctaagacggctcctggaaagaagagaccggtaga |
| | | gccatcaccccagcgttctccagactcctctacgggcatcggcaagaaaggccag |
| | | cagcccgcgaaaaagagactcaactttgggcagactggcgactcagagtcagtgc |
| | | ccgaccctcaaccaatcggagaaccccccgcaggcccctctggtctgggatctgg |
| | | tacaatggctgcaggcggtggcgctccaatggcagacaataacgaaggcgccgac |
| | | ggagtgggtagttcctcaggaaattggcattgcgattccacatggctgggcgaca |
| | | gagtcatcaccaccagcacccgaacctgggccctccccacctacaacaaccacct |
| | | ctacaagcaaatctccaacgggacttcgggaggaagcaccaacgacaacacctac |
| | | ttcggctacagcaccccctgggggtattttgactttaacagattccactgccact |
| | | tctcaccacgtgactggcagcgactcatcaacaacaactggggattccggcccaa |
| | | gagactcaacttcaagctcttcaacatccaggtcaaggaggtcacgcagaatgaa |
| | | ggcaccaagaccatcgccaataaccttaccagcacgattcaggtctttacggact |
| | | cggaataccagctcccgtacgtcctcggctctgcgcaccagggctgcctgcctcc |
| | | gttcccggcggacgtcttcatgattcctcagtacgggtacctgactctgaacaat |
| | | ggcagtcaggccgtgggccgttcctccttctactgcctggagtactttccttctc |
| | | aaatgctgagaacgggcaacaactttgagttcagctaccagtttgaggacgtacc |
| | | tttccatagcagctacgctcacagccaaagcctggaccgactaatgaatccactc |
| | | atcgaccaatacttgtactatctctcaaagactattaacggttctggacagaatc |
| | | aacaaacgctaaaattcagtgtggccggacccagcaacatggctgtccagggaag |
| | | aaactacatacctggacccagctaccgacaacaacgtgtctcaaccactgtgact |
| | | caaaacaacaacagcgaatttgcttggcctggagcttcttcttgggctctcaatg |
| | | gacgtaatagcttgatgaatcctggacctgctatggccagccacaaagaaggaga |
| | | ggaccgtttctttcctttgtctggatctttaatttttggcaaacaaggaactgga |
| | | agagacaacgtggatgcggacaaagtcatgataaccaacgaagaagaaattaaaa |
| | | ctactaacccggtagcaacggagtcctatggacaagtggccacaaaccaccagag |
| | | tgcccaagcacaggcgcagaccggctgggttcaaaaccaaggaatacttccgggt |
| | | atggtttggcaggacagagatgtgtacctgcaaggacccatttgggccaaaattc |
| | | ctcacacggacggcaactttcacccttctccgctgatgggagggtttggaatgaa |
| | | gcacccgcctcctcagatcctgattaagaatacacctgttcccgcggatcctcca |
| | | actaccttcagtcaagctaagctggcgtcgttcatcacgcagtacagcaccggac |
| | | aggtcagcgtggaaattgaatgggagctgcagaaagaaaacagcaaacgctggaa |
| | | cccagagattcaatacacttccaactactacaaatctacaaatgtggactttgct |
| | | gttaacacagatggcacttattctgagcctcgccccatcggcacccgttacctca |
| | | cccgtaatctgtaa |
| 43 | AAV5/bovine | msfvdhppdwleevgeglreflgleagppkpkpnqqhqdqarglvlpgynylgpg |
| | AAV GH loop | ngldrgepvnradevarehdisyneqleagdnpylkynhadaefqekladdtsfg |
| | VP1 amino | gnlgkavfqakkrvlepfglveegaktaptgkriddhfpkrkkarteedskpsts |
| | acid | sdaeagpsgsqqlqipaqpasslgadtmsaggggplgdnnqgadgvgnasgdwhc |
| | sequence (GH | dstwmgdrvvtkstrtwvlpsynnhqyreiksgsvdgsnanayfgystpwgyfdf |
| | loop region | nrfhshwsprdwqrlinnywgfrprslrvkifniqvkevtvqdstttiannltst |
| | underlined) | vqvftdddyqlpyvvgngtegclpafppqvftlpqygyatlnrdntenpterssf |
| | | fcleyfpskmlrtgnnfemvykfenvpfhsmyahsqsldrlmnplldqylwelqs |
| | | ttsggtlnqgnsatnfakltktnfsgyrknwlpgpmmkqqrfsktasqnykipqg |
| | | rnnsllhyetrttldgrwsnfapgtamataandatdfsqaqlifagpnitgnttt |
| | | dannlmftsedelratnprdtdlfghlatnqqnattvptvddvdgvgvypgmvwq |
| | | drdiyyqgpiwakiphtdghfhpspliggfglkspppqifikntpvpanpattfs |
| | | parinsfitqystgqvtvemewelkkenskrwnpeiqytnnyndpqfvdfapdst |
| | | geyrttrpigtryltrpl |
| 44 | AAV5/bovine | atgtcttttgttgatcaccctccagattggttggaagaagttggtgaaggtcttc |
| | AAV GH loop | gcgagtttttgggccttgaagcgggcccaccgaaaccaaaacccaatcagcagca |
| | VP1 | tcaagatcaagcccgtggtcttgtgctgcctggttataactatctcggacccgga |
| | nucleotide | aacggtctcgatcgaggagagcctgtcaacagggcagacgaggtcgcgcgagagc |
| | sequence | acgacatctcgtacaacgagcagcttgaggcgggagacaacccctacctcaagta |
| | | caaccacgcggacgccgagtttcaggagaagctcgccgacgacacatccttcggg |
| | | ggaaacctcggaaaggcagtctttcaggccaagaaaagggttctcgaaccttttg |
| | | gcctggttgaagagggtgctaagacggcccctaccggaaagcggatagacgacca |
| | | ctttccaaaaagaaagaaggctcggaccgaagaggactccaagccttccacctcg |
| | | tcagacgccgaagctggacccagoggatcccagcagctgcaaatcccagcccaac |
| | | cagcctcaagtttgggagctgatacaatgtctgcgggaggtggcggcccattggg |
| | | cgacaataaccaaggtgccgatggagtgggcaatgcctcgggagattggcattgc |
| | | gattccacgtggatgggggacagagtcgtcaccaagtccacccgaacctgggtgc |
| | | tgcccagctacaacaaccaccagtaccgagagatcaaaagcggctccgtcgacgg |
| | | aagcaacgccaacgcctactttggatacagcaccccctgggggtactttgacttt |
| | | aaccgcttccacagccactggagcccccgagactggcaaagactcatcaacaact |
| | | actggggcttcagaccccggtccctcagagtcaaaatcttcaacattcaagtcaa |
| | | agaggtcacggtgcaggactccaccaccaccatcgccaacaacctcacctccacc |
| | | gtccaagtgtttacggacgacgactaccagctgccctacgtcgtcggcaacggga |
| | | ccgagggatgcctgccggccttccctccgcaggtctttacgctgccgcagtacgg |
| | | ttacgcgacgctgaaccgcgacaacacagaaaatcccaccgagaggagcagcttc |
| | | ttctgcctagagtactttcccagcaagatgctgagaacgggcaacaactttgaga |
| | | tggtgtacaagtttgaaaacgtgcccttccactccatgtacgctcacagccagag |
| | | cctggataggctgatgaacccgctgctggaccagtacctgtgggagctccagtct |
| | | accacctctggaggaactctcaaccagggcaattcagccaccaactttgccaagc |
| | | tgaccaaaacaaacttttctggctaccgcaaaaactggctcccggggcccatgat |
| | | gaagcagcagagattctccaagactgccagtcaaaactacaagattccccaggga |
| | | agaaacaacagtctgctccattatgagaccagaactaccctcgacggaagatgga |
| | | gcaattttgccccgggaacggccatggcaaccgcagccaacgacgccaccgactt |
| | | ctctcaggcccagctcatctttgcggggcccaacatcaccggcaacaccaccaca |
| | | gatgccaataacctgatgttcacttcagaagatgaacttagggccaccaaccccc |
| | | gggacactgacctgtttggccacctggcaaccaaccagcaaaacgccaccaccgt |
| | | tcctaccgtagacgacgtggacggagtcggcgtgtacccgggaatggtgtggcag |
| | | gacagagacatttactaccaagggcccatttgggccaaaattccacacacggatg |
| | | gacactttcacccgtctcctctcattggcggatttggactgaaaagcccgcctcc |
| | | acaaatattcatcaaaaacactcctgtacccgccaatcccgcaacgaccttctct |
| | | ccggccagaatcaacagcttcatcacccagtacagcaccgggcaggtcaccgtgg |
| | | agatggagtgggagctcaagaaggaaaactccaagaggtggaacccagagatcca |
| | | gtacacaaacaactacaacgacccccagtttgtggactttgccccggacagcacc |
| | | ggggaatacagaaccaccagacctatcggaacccgataccttacccgaccccttt |
| | | aa |
| 45 | AAV5/porcine | msfvdhppdwleevgeglreflgleagppkpkpnqqhqdqarglvlpgynylgpg |
| | 4 AAV GH | ngldrgepvnradevarehdisyneqleagdnpylkynhadaefqekladdtsfg |
| | loop VP1 | gnlgkavfqakkrvlepfglveegaktaptgkriddhfpkrkkarteedskpsts |
| | amino acid | sdaeagpsgsqqlqipaqpasslgadtmsaggggplgdnnqgadgvgnasgdwhc |
| | sequence (GH | dstwmgdrvvtkstrtwvlpsynnhqyreiksgsvdgsnanayfgystpwgyfdf |
| | loop region | nrfhshwsprdwqrlinnywgfrprslrvkifniqvkevtvqdstttiannltst |
| | underlined) | vqvftdddyqlpyvvgngtegclpafppqvftlpqygyatlnrdntenpterssf |
| | | fcleyfpskmlrtgnnftfsytfedvpfhssyahsqsldrlmnplidqylyylsk |
| | | tagglafsqagpttmrnqsrnwlpgpcfrqqrvserstennngdfswtgttryhl |
| | | ngrdsamnpgpamashkddedrffpqngvlifgtpnatasnaplenvlitdeeei |
| | | rttnpvateeygivannkqdsstqattaivnaqgilpgmvwqdrdvylqgpiwak |
| | | iphtdghfhpsplmggfglkhpppqilikntpvpsnppekftqeklnafitqyst |
| | | gqvtvemewelkkenskrwnpeiqytnnyndpqfvdfapdstgeyrttrpigtry |
| | | ltrpl |
| 46 | AAV5/porcine | atgtcttttgttgatcaccctccagattggttggaagaagttggtgaaggtcttc |
| | 4 AAV GH | gcgagtttttgggccttgaagcgggcccaccgaaaccaaaacccaatcagcagca |
| | loop VP1 | tcaagatcaagcccgtggtcttgtgctgcctggttataactatctcggacccgga |
| | nucleotide | aacggtctcgatcgaggagagcctgtcaacagggcagacgaggtcgcgcgagagc |
| | sequence | acgacatctcgtacaacgagcagcttgaggcgggagacaacccctacctcaagta |
| | | caaccacgcggacgccgagtttcaggagaagctcgccgacgacacatccttcggg |
| | | ggaaacctcggaaaggcagtctttcaggccaagaaaagggttctcgaaccttttg |
| | | gcctggttgaagagggtgctaagacggcccctaccggaaagcggatagacgacca |
| | | ctttccaaaaagaaagaaggctcggaccgaagaggactccaagccttccacctcg |
| | | tcagacgccgaagctggacccagcggatcccagcagctgcaaatcccagcccaac |
| | | cagcctcaagtttgggagctgatacaatgtctgcgggaggtggcggcccattggg |
| | | cgacaataaccaaggtgccgatggagtgggcaatgcctcgggagattggcattgc |
| | | gattccacgtggatgggggacagagtcgtcaccaagtccacccgaacctgggtgc |
| | | tgcccagctacaacaaccaccagtaccgagagatcaaaagcggctccgtcgacgg |
| | | aagcaacgccaacgcctactttggatacagcaccccctgggggtactttgacttt |
| | | aaccgcttccacagccactggagcccccgagactggcaaagactcatcaacaact |
| | | actggggcttcagaccccggtccctcagagtcaaaatcttcaacattcaagtcaa |
| | | agaggtcacggtgcaggactccaccaccaccatcgccaacaacctcacctccacc |
| | | gtccaagtgtttacggacgacgactaccagctgccctacgtcgtcggcaacggga |
| | | ccgagggatgcctgccggccttccctccgcaggtctttacgctgccgcagtacgg |
| | | ttacgcgacgctgaaccgcgacaacacagaaaatcccaccgagaggagcagcttc |
| | | ttctgcctagagtactttcccagcaagatgctgagaacgggcaacaactttacgt |
| | | tcagctacacctttgaggacgtgcccttccacagcagctacgcgcacagccagag |
| | | cctggaccggctgatgaacccgctcatcgaccagtacctgtactacctgagcaag |
| | | acagccggtggtctggcgttttctcaagcagggcccaccaccatgcggaaccagt |
| | | ccaggaactggctcccgggaccctgcttccgacagcagcgagtttcagaaagatc |
| | | cacagaaaacaacaacggagacttctcgtggacagggaccacgagataccatctt |
| | | aacggtagagactcggcgatgaatccgggcccggccatggccagtcacaaggacg |
| | | acgaggacagattctttccgcaaaacggcgtgctcatctttggaacaccgaacgc |
| | | tactgcaagcaatgcacctctagagaatgtactgatcaccgatgaggaggaaatc |
| | | agaactacgaaccctgtagccacggaagagtacgggatcgttgctaacaacaaac |
| | | aggacagcagtacacaggcgacgaccgcgattgtcaacgcacaaggcatactgcc |
| | | cggcatggtgtggcaggatcgagatgtgtacctgcaggggcccatctgggccaag |
| | | atcccccacacggatggacacttccacccatcaccgctcatgggcgggtttggcc |
| | | ttaagcatccacctccgcagatcctcatcaagaacacgcctgtgccgtcgaaccc |
| | | tccagaaaagttcacccaggaaaaactaaatgccttcatcacccagtacagcacc |
| | | gggcaggtcaccgtggagatggagtgggagctcaagaaggaaaactccaagaggt |
| | | ggaacccagagatccagtacacaaacaactacaacgacccccagtttgtggactt |
| | | tgccccggacagcaccggggaatacagaaccaccagacctatcggaacccgatac |
| | | cttacccgacccctttaa |
| 47 | AAV5/porcine | msfvdhppdwleevgeglreflgleagppkpkpnqqhqdqarglvlpgynylgpg |
| | 5 GH loop | ngldrgepvnradevarehdisyneqleagdnpylkynhadaefqekladdtsfg |
| | VP1 amino | gnlgkavfqakkrvlepfglveegaktaptgkriddhfpkrkkarteedskpsts |
| | acid | sdaeagpsgsqqlqipaqpasslgadtmsaggggplgdnnqgadgvgnasgdwhc |
| | sequence (GH | dstwmgdrvvtkstrtwvlpsynnhqyreiksgsvdgsnanayfgystpwgyfdf |
| | loop region | nrfhshwsprdwqrlinnywgfrprslrvkifniqvkevtvqdstttiannltst |
| | underlined) | vqvftdddyqlpyvvgngtegclpafppqvftlpqygyatlnrdntenpterssf |
| | | fcleyfpskmlrtgnnfeftynfeevpfhssfapsqnlfklanplvdqylyrfvs |
| | | tdtsgavqfkknlagryantyknwfpgpmcrtqgwytgtgiynnkgatsfntsnr |
| | | mdlegasyqvppqpngmtntvqdsnlyalentmifnaqnatpgtnttypeenlli |
| | | tsesetqpvnrvayssggqiannnqntntaptagtynhqeilpgsvwmdrdvylq |
| | | gpiwakipetgahfhpspamggfglkypppmmlikntpvpgnitsfsdvpvssfi |
| | | tqystgqvtvemewelkkenskrwnpeiqytnnyndpqfvdfapdstgeyrttrp |
| | | igtryltrpl |
| 48 | AAV5/porcine | atgtcttttgttgatcaccctccagattggttggaagaagttggtgaaggtcttc |
| | 5 GH loop | gcgagtttttgggccttgaagcgggcccaccgaaaccaaaacccaatcagcagca |
| | VP1 | tcaagatcaagcccgtggtcttgtgctgcctggttataactatctcggacccgga |
| | nucleotide | aacggtctcgatcgaggagagcctgtcaacagggcagacgaggtcgcgcgagagc |
| | sequence | acgacatctcgtacaacgagcagcttgaggcgggagacaacccctacctcaagta |
| | | caaccacgcggacgccgagtttcaggagaagctcgccgacgacacatccttcggg |
| | | ggaaacctcggaaaggcagtctttcaggccaagaaaagggttctcgaaccttttg |
| | | gcctggttgaagagggtgctaagacggcccctaccggaaagcggatagacgacca |
| | | ctttccaaaaagaaagaaggctcggaccgaagaggactccaagccttccacctcg |
| | | tcagacgccgaagctggacccagoggatcccagcagctgcaaatcccagcccaac |
| | | cagcctcaagtttgggagctgatacaatgtctgcgggaggtggcggcccattggg |
| | | cgacaataaccaaggtgccgatggagtgggcaatgcctcgggagattggcattgc |
| | | gattccacgtggatgggggacagagtcgtcaccaagtccacccgaacctgggtgc |
| | | tgcccagctacaacaaccaccagtaccgagagatcaaaagcggctccgtcgacgg |
| | | aagcaacgccaacgcctactttggatacagcaccccctgggggtactttgacttt |
| | | aaccgcttccacagccactggagcccccgagactggcaaagactcatcaacaact |
| | | actggggcttcagaccccggtccctcagagtcaaaatcttcaacattcaagtcaa |
| | | agaggtcacggtgcaggactccaccaccaccatcgccaacaacctcacctccacc |
| | | gtccaagtgtttacggacgacgactaccagctgccctacgtcgtcggcaacggga |
| | | ccgagggatgcctgccggccttccctccgcaggtctttacgctgccgcagtacgg |
| | | ttacgcgacgctgaaccgcgacaacacagaaaatcccaccgagaggagcagcttc |
| | | ttctgcctagagtactttcccagcaagatgctgagaacgggcaacaactttgagt |
| | | ttacctacaactttgaggaggtgcccttccactccagcttcgctcccagtcagaa |
| | | cctgttcaagctggccaacccgctggtggatcagtacctttaccgcttcgtgagc |
| | | acggacacctccggtgccgtccagttcaaaaagaacctggcgggcagatacgcca |
| | | acacctacaagaactggttcccaggacccatgtgccgaacccagggctggtacac |
| | | aggaacgggtatatataacaacaaaggcgctaccagctttaacacctcaaacaga |
| | | atggacctagagggagccagttatcaagtgcctccccagcccaacgggatgacaa |
| | | acacggttcaggacagcaacctttacgcgctggaaaacaccatgatttttaacgc |
| | | acaaaacgccaccccgggaacgaatacaacgtatccggaggagaaccttttgata |
| | | accagtgagagcgagactcaacccgtgaacagagtggcttacagctccggaggac |
| | | aaatagccaacaacaatcagaataccaacacggctcctactgcaggaacctacaa |
| | | ccaccaggaaatactacctggcagcgtgtggatggacagggacgtgtacctccag |
| | | ggtcccatctgggccaagatcccagagacaggggcacactttcatccttctccag |
| | | ccatgggcgggttcggactcaaatacccgcctcccatgatgctaatcaagaacac |
| | | gccagtgcccggaaatatcaccagcttctcggacgtgcccgtcagcagcttcatc |
| | | acccagtacagcaccgggcaggtcaccgtggagatggagtgggagctcaagaagg |
| | | aaaactccaagaggtggaacccagagatccagtacacaaacaactacaacgaccc |
| | | ccagtttgtggactttgccccggacagcaccggggaatacagaaccaccagacct |
| | | atcggaacccgataccttacccgacccctttaa |
| 49 | β-sheet G | FTFSYT |
| | AAV2 | |
| 50 | β-sheet G | FEITYS |
| | AAV4 | |
| 51 | β-sheet G | FQFTYT |
| | AAV8 | |
| 52 | β-sheet G | FX₁X₂X₃YX₄, wherein X₁ is E, Q, or T; wherein X₂ is F, I, |
| | consensus | or M; wherein X₃ is S, T or V; and X₄ is E, K, N, Q, S |
| | | or T |
| 53 | β-sheet G | FX₁X₂X₃YX₄, wherein X₁ is E, Q, or T; wherein X₂ is F or |
| | consensus | I; wherein X₃ is S or T; and X₄ is E, N, Q, S or T |
| 54 | β-sheet G | FQFSYT |
| | AAV3B | |
| 55 | β-sheet G | FEFTYN |
| | AAV5 | |
| 56 | β-sheet G | FTFSYT |
| | AAV6 | |
| 57 | β-sheet G | FEFSYS |
| | AAV7 | |
| 58 | β-sheet G | FQFSYE |
| | AAV9 | |
| 59 | β-sheet G | FEFSYQ |
| | AAVrh10 | |
| 60 | β-sheet G | FEMVYK |
| | AAVbovine | |
| 61 | β-sheet G | FTFSYT |
| | AAVporcine4 | |
| 62 | β-sheet G | FEFTYS |
| | AAVporcine5 | |
| 63 | β-sheet H | QILIKNT |
| | AAV2, 6, 7, | |
| | 8, 9, rh10, | |
| | bovine, | |
| | porcine4 | |
| 64 | β-sheet H | QIFIKNT |
| | AAV4, bovine | |
| 65 | β-sheet H | X₁X₂X₃IKNT, wherein X₁ is Q, or M; wherein X₂ is I or M; |
| | consensus | and wherein X₃ is L, M or F |
| 66 | β-sheet H | QX₁X₂IKNT, wherein X₁ is I or M; and wherein X₂ is F, L |
| | consensus | or M |
| 67 | β-sheet H | QIMIKNT |
| | AAV3B | |
| 68 | β-sheet H | MMLIKNT |
| | AAV5, | |
| | porcine5 | |
| 69 | β-sheet I | TQYSTGQVSVEIEWELQ |
| | AAV2, 3B, 6, | |
| | 7, 8, 9, | |
| | rh10, | |
| | porcine4 | |
| 70 | β-sheet I | TQYSTGQVSVQIDWEIQ |
| | AAV4 | |
| 71 | β-sheet I | TQYSTGQVX₁VX₂X₃X₄WEX₅X₆, wherein X₁ is A, S or T; wherein |
| | consensus | X₂ is E, K or Q; wherein X₃ is I or M; wherein X₄ is D |
| | | or E; wherein X₅ is I or L; and wherein X₆ is Q or K |
| 72 | β-sheet I | TOYSTGQVX₁VX₂X₃EWEX₄X₅, wherein X₁ is A, S or T; wherein |
| | consensus | X₂ is E or K; wherein X₃ is I or M; wherein X₄ is I or |
| | | L; and wherein X₅ is Q or K |
| 73 | β-sheet I | TQYSTGQVTVEMEWELK |
| | consensus | |
| | AAV5, | |
| | porcine5 | |
| 74 | β-sheet I | TQYSTGQVAVKIEWEIQ |
| | consensus | |
| | AAVbovine | |
| 75 | | VPFHS |
| 76 | | KHPPP |
| 77 | | MKHPPP |
| 78 | | GNNFX₁F wherein X1 is E, Q or T |
| 79 | | LYRFVST |
| 80 | | PPPM |
| 81 | | FX₁X₂X₃YX₄, wherein X₁ is E or T; wherein X₂ is F or M; |
| | | wherein X₃ is S, T or V; and wherein X₄ is K, N or T |
| 82 | | MLRTGNNF |
| 83 | | FITQYSTGQV |

1. An AAV capsid polypeptide comprising an amino acid sequence of a parental AAV VP1 polypeptide, comprising a substitution of amino acids from a region between β-sheet G and β-sheet H with amino acids from a region between β-sheet G and β-sheet H of an alternative AAV VP1 polypeptide.

2. The AAV capsid polypeptide of claim 1, wherein the region between β-sheet G and β-sheet H of the parental AAV VP1polypeptide and the alternative AAV VP1 polypeptide comprises about 222 to about 235 amino acids.

3. The AAV capsid polypeptide of claim 1, wherein β-sheet G of the parental AAV VP1 polypeptide and β-sheet G of the alternative AAV VP1 polypeptide comprises or consists of an amino acid sequence of FX₁X₂X₃YX₄, wherein X₁ is E, Q, or T; wherein X₂ is F, I, or M; wherein X₃ is S, T or V; and wherein X₄ is E, K, N, Q, S or T (SEQ ID NO:52) or, wherein β-sheet H of the parental AAV VP1 polypeptide and β-sheet H of the alternative AAV VP1 polypeptide comprises or consists of an amino acid sequence of X₁X₂X₃IKNT, wherein X₁ is Q, or M; wherein X₂ is I or M; and wherein X₃ is L, M or F (SEQ ID NO: 65).

4. (canceled)

5. The AAV capsid polypeptide of claim 1, wherein a serotype of the parental AAV VP1 polypeptide is selected from the group consisting of AAV2, AAV3B, AAV4, AAV5, AAV6, AAV7, AAV8, AAV9, AAVrh10, AAVbovine, AAVporcine4 and AAVporcine5, and/or wherein a serotype of the alternative AAV VP1 polypeptide is selected from the group consisting of AAV2, AAV3B, AAV4, AAV5, AAV6, AAV7, AAV8, AAV9, AAVrh10, AAVbovine, AAVporcine4 and AAVporcine5.

6. (canceled)

7. The AAV capsid polypeptide of claim 1, wherein the amino acid sequence of the parental AAV VP1 polypeptide, of the alternative AAV VP1 polypeptide or both is set forth in any one of SEQ ID NO:1, SEQ ID NO:3, SEQ ID NO:5, SEQ ID NO:7, SEQ ID NO:9, SEQ ID NO:11, SEQ ID NO:13, SEQ ID NO: 15, SEQ ID NO:17, SEQ ID NO:19, SEQ ID NO:21 and SEQ ID NO: 23 or, wherein the amino acid sequence of the parental AAV VP1 polypeptide, of the alternative AAV VP1 polypeptide or both is encoded by a nucleic acid set forth in any one of SEQ ID NO:2, SEQ ID NO:4, SEQ ID NO: 6, SEQ ID NO:8, SEQ ID NO:10, SEQ ID NO:12, SEQ ID NO:14, SEQ ID NO:16, SEQ ID NO:18, SEQ ID NO: 20, SEQ ID NO:22 and SEQ ID NO:24.

8. (canceled)

9. The AAV capsid polypeptide of claim 1, wherein the region between β-sheet G and β-sheet H of the parental AAV VP1 polypeptide, the region between β-sheet G and β-sheet H of the alternative AAV VP1 polypeptide or both is flanked by an amino acid sequence comprising or consisting of the sequence VPFHS, (SEQ ID NO:75) at the amino terminal end and is flanked by an amino acid sequence comprising or consisting of the sequence KHPPP (SEQ ID NO:76) or MKHPPP (SEQ ID NO: 77) at the carboxy terminal end or, wherein the serotype of the parental AAV VP1 polypeptide is AAV9 or AAVrh10 and wherein the serotype of the alternative AAV VP1 polypeptide is selected from the group consisting of AAV2, AAV3B, AAV4, AAV5 and AAV9.

10. (canceled)

11. The AAV capsid polypeptide of claim 9, wherein the polypeptide comprises an amino acid sequence at least 90%, 95%, 98% or 99% identical to an amino acid sequence of any one of SEQ ID NO:25, SEQ ID NO:27, SEQ ID NO:29, SEQ ID NO:31, SEQ ID NO:39, and SEQ ID NO:41 or comprises or consists of the amino acid sequence of any one of SEQ ID NO:25, SEQ ID NO: 27, SEQ ID NO:29, SEQ ID NO:31, SEQ ID NO:39, and SEQ ID NO:41.

12. (canceled)

13. The AAV capsid polypeptide of claim 1, wherein the region between β-sheet G and β-sheet H of the parental AAV VP1 polypeptide, the region between β-sheet G and β-sheet H of the alternative AAV VP1 polypeptide or both is flanked by an amino acid sequence comprising or consisting of the sequence GNNFX₁F wherein X1 is E, Q or T (SEQ ID NO:78) at the amino terminal end and flanked by an amino acid sequence comprising or consisting of the sequence KHPPP (SEQ ID NO: 76) at the carboxy terminal end or, wherein the serotype of the parental AAV VP1 polypeptide is AAV9 and wherein the serotype of the alternative AAV VP1 polypeptide is selected from the group consisting of AAV6, AAV7 and AAV8.

14. (canceled)

15. The AAV capsid polypeptide of claim 13, wherein the polypeptide comprises an amino acid sequence at least 90%, 95%, 98% or 99% identical to any one of SEQ ID NO:33, SEQ ID NO: 35 and SEQ ID NO:37, or wherein the polypeptide comprises or consists of the amino acid sequence of any one of SEQ ID NO:33, SEQ ID NO:35 and SEQ ID NO:37.

16. (canceled)

17. The AAV capsid polypeptide of claim 1, wherein the region between β-sheet G and β-sheet H of the parental AAV VP1polypeptide, the region between β-sheet G and β-sheet H of the alternative AAV VP1 polypeptide or both is flanked by an amino acid sequence comprising or consisting of the sequence LYRFVST (SEQ ID NO:79) at the amino terminal end and flanked by an amino acid sequence comprising or consisting of the sequence PPPM (SEQ ID NO:80) at the carboxy terminal end or, wherein the serotype of the parental AAV VP1 polypeptide is AAV5 and wherein the serotype of the alternative AAV VP1 polypeptide is AAVporcine5.

18. (canceled)

19. The AAV capsid polypeptide of claim 17, wherein the polypeptide comprises an amino acid sequence at least 90%, 95%, 98% or 99% identical to SEQ ID NO:47, or comprises or consists of the amino acid sequence of SEQ ID NO:47.

20. (canceled)

21. An AAV capsid polypeptide comprising an amino acid sequence of a parental AAV VP1 polypeptide, comprising a substitution of amino acids from a region between β-sheet G and β-sheet I with amino acids from a region between β-sheet G and β-sheet I of an alternative AAV VP1 polypeptide.

22. The AAV capsid polypeptide claim 21, wherein the β-sheet G of the parental AAV VP1 polypeptide and of the alternative AAV VP1 polypeptide comprises or consists of an amino acid sequence of FX₁X₂X₃YX₄, wherein X₁ is E or T; wherein X₂ is F or M; wherein X₃ is S, T or V; and wherein X₄ is K, N or T (SEQ ID NO:81) or, wherein the parental AAV VP1, the alternative AAV VP1 or both is selected from the group consisting of AAV2, AAV5, AAV6, AAV8, AAVbovine, AAVporcine4 and AAVporcine5.

23. (canceled)

24. The AAV capsid polypeptide of claim 21, wherein the β-sheet I of the parental AAV VP1 polypeptide and of the alternative AAV VP1 polypeptide comprises or consists of an amino acid sequence of TQYSTGQVX₁VX₂X₃X₄WEX₅X₆, wherein X₁ is A, S or T; wherein X₂ is E, K or Q; wherein X₃ is I or M; wherein X₄ is D or E; wherein X₅ is I or L; and wherein X₆ is Q or K (SEQ ID NO: 71) or, wherein the parental AAV VP1, the alternative AAV VP1 or both is selected from the group consisting of AAV2, AAV3B, AAV4, AAV5, AAV6, AAV7, AAV8, AAV9, AAVrh10, AAVbovine, AAVporcine4 and AAVporcine5.

25. (canceled)

26. The AAV capsid polypeptide of claim 21, wherein the amino acid sequence of the parental AAV VP1 polypeptide, of the alternative AAV VP1 polypeptide, or both is set forth in any one of SEQ ID NO:1, SEQ ID NO:3, SEQ ID NO:7, SEQ ID NO:9, SEQ ID NO:11, SEQ ID NO: 13, SEQ ID NO: 15, SEQ ID NO: 17, SEQ ID NO: 19, SEQ ID NO:21 and SEQ ID NO:23 or, wherein the amino acid sequence of the parental AAV VP1 polypeptide, of the alternative AAV VP1 polypeptide, or both is encoded by a nucleic acid set forth in any one of SEQ ID NO:2, SEQ ID NO:4, SEQ ID NO:8, SEQ ID NO: 10, SEQ ID NO:12, SEQ ID NO:14, SEQ ID NO:16, SEQ ID NO:18, SEQ ID NO: 20, SEQ ID NO:22 and SEQ ID NO:24.

27. (canceled)

28. The AAV capsid polypeptide of claim 21, wherein the region between β-sheet G and β-sheet I of the parental AAV VP1polypeptide and the region between β-sheet G and β-sheet I of the alternative AAV VP1 polypeptide is flanked by an amino acid sequence comprising or consisting of the sequence MLRTGNNF (SEQ ID NO:82) at the amino terminal end and flanked by an amino acid sequence comprising or consisting of the sequence FITQYSTGQV (SEQ ID NO:83) at the carboxy terminal end or, wherein the serotype of the parental AAV VP1 polypeptide is AAV5 and wherein the serotype of the alternative AAV VP1 polypeptide is AAVbovine or AAVporcine4.

29. (canceled)

30. The AAV capsid polypeptide of claim 21, wherein the polypeptide comprises an amino acid sequence at least 90%, 95%, 98% or 99% identical to SEQ ID NO:43 or SEQ ID NO: 45, or wherein the polypeptide comprises or consists of the amino acid sequence of SEQ ID NO: 43 or SEQ ID NO:45.

31. (canceled)

32. An AAV capsid polypeptide comprising an amino acid sequence of an AAV9 VP1 polypeptide, comprising a substitution of amino acids from a region between β-sheet G and β-sheet H with amino acids from a region between β-sheet G and β-sheet H of an AAV6 VP1 polypeptide, and wherein the polypeptide comprises the amino acid sequence of SEQ ID NO:33.

33. The AAV capsid polypeptide of claim 32, wherein any one or more of amino acids E547 S552, N553, A555, N558 V559 from the AAV6 VP1 polypeptide and wherein any one or more of amino acids N710, N711 and V721 from the AAV9 VP1 polypeptide interact with the PKD1 receptor or, wherein any one or more of amino acids W504 and T505 from the AAV6 VP1 polypeptide and wherein any one or more of amino acids S263, S269, S386, Q387, D384, N270 from the AAV9 VP1 polypeptide interact with the PKD2 receptor.

34. (canceled)

35. An AAV capsid polypeptide comprising or consisting of an amino acid sequence selected from the group consisting of SEQ ID NO:25, SEQ ID NO:27, SEQ ID NO:29, SEQ ID NO:31, SEQ ID NO: 33, SEQ ID NO:35, SEQ ID NO:37, SEQ ID NO:39, SEQ ID NO:41, SEQ ID NO:43, SEQ ID NO:45 and SEQ ID NO:47 or an AAV capsid polypeptide comprising or consisting of an amino acid sequence encoded by a nucleic acid sequence selected from the group consisting of SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO: 42, SEQ ID NO:44, SEQ ID NO:46 and SEQ ID NO:48.

36. (canceled)

37. A recombinant adeno-associated viral (rAAV) vector comprising an AAV capsid polypeptide of claim 1.

38. (canceled)

39. The rAAV vector of claim 37 or 38, wherein the rAAV vector comprises a nucleic acid comprising a transgene, and wherein the transgene encodes a therapeutic protein or a reporter protein.

40. The rAAV vector of claim 37, wherein the tropism of the vector differs from the tropism of an otherwise identical rAAV vector comprising an AAV capsid polypeptide of a parental serotype.

41. A nucleic acid encoding the AAV capsid polypeptide of claim 1.

42. (canceled)

43. A host cell comprising a nucleic acid encoding the AAV capsid polypeptide of claim 1.

44. (canceled)

45. A pharmaceutical composition comprising i) a rAAV vector comprising an AAV capsid polypeptide of claim 1 and a vector genome and ii) a pharmaceutically acceptable excipient.

46. A pharmaceutical composition comprising i) a rAAV vector of claim 37 and ii) a pharmaceutically acceptable excipient.

47. A method of making a rAAV vector, the method comprising i) transfecting a host cell in culture with a plasmid comprising a nucleic acid encoding an AAV capsid polypeptide of claim 1, and a plasmid comprising a nucleic acid encoding a therapeutic protein or a reporter protein and ii) isolating the rAAV vector from the host cell, from the culture media or both.

48. (canceled)

49. (canceled)

50. (canceled)

51. (canceled)

52. (canceled)

53. (canceled)

54. (canceled)

55. A method of transducing a target cell, the method comprising contacting the target cell with a rAAV vector comprising an AAV capsid polypeptide of claim 1 such that the rAAV vector is introduced into the target cell.

56. The method of claim 55, wherein the target cell is a liver (e.g., hepatocytes, sinusoidal endothelial cells), pancreas (e.g., beta islet cells, exocrine), lung, central or peripheral nervous system, such as brain (e.g., neural or ependymal cells, oligodendrocytes) or spine, kidney, eye (e.g., retinal), spleen, skin, thymus, testes, lung, diaphragm, heart (cardiac), muscle or psoas, or gut (e.g., endocrine), adipose tissue (white, brown or beige), muscle (e.g., fibroblasts, myocytes), synoviocytes, chondrocytes, osteoclasts, epithelial cells, endothelial cells, salivary gland cells, inner ear nervous cells, hematopoietic (e.g., blood or lymph) or stem (e.g., pluripotent or multipotent progenitor) cell.

57. (canceled)

58. (canceled)

59. (canceled)

60. (canceled)

61. (canceled)

62. (canceled)
